(19) United States
(12) Patent Application Publication
(10) Pub. No.: US 2024/0262931 A1
(43) Pub. Date: Aug. 8, 2024

(54) PRECURSOR PROTEINS AND KIT FOR TARGETED THERAPY

(71) Applicant: Hoffmann-La Roche Inc., Little Falls, NJ (US)

(72) Inventors: Ulrich BRINKMANN, Weilheim (DE); Alexander BUJOTZEK, München (DE); Can BULDUN, Penzberg (DE); Guy GEORGES, Habach (DE)

(73) Assignee: Hoffmann-La Roche Inc., Little Falls, NJ (US)

(21) Appl. No.: 18/335,515

(22) Filed: Jun. 15, 2023

Related U.S. Application Data
(63) Continuation of application No. PCT/EP2021/086151, filed on Dec. 16, 2021.

(30) Foreign Application Priority Data
Dec. 18, 2020 (EP) 20215328.4

(51) Int. Cl. C07K 16/32; C07K 14/54; C07K 14/55; C07K 16/28; C07K 19/00

(52) U.S. Cl. CPC C07K 16/32; C07K 14/5406; C07K 14/5434; C07K 14/55; C07K 16/2896; C07K 19/00; C07K 2317/526

(57) ABSTRACT

The present invention relates to a set of precursor proteins comprising an inactivated receptor ligand or enzyme and methods for their use in therapy.

### CROSS-REFERENCE TO RELATED APPLICATION

This application is a continuation of International Patent Application No. PCT/EP2021/086151 filed Dec. 16, 2021, which claims the benefit of EP Patent Application No. 20215328.4, filed Dec. 18, 2020, the contents of which are incorporated by reference herein in their entireties.

### SEQUENCE LISTING

The instant application contains a Sequence Listing submitted electronically in XML format and is hereby incorporated herein by reference in its entirety. Said XML copy, created Jun. 6, 2023, is named P36624-US.xml and is 76,766 bytes in size.

### FIELD OF THE INVENTION

The present invention relates to a set of precursor proteins comprising an inactivated receptor ligand or enzyme and methods for their use in therapy.

### BACKGROUND OF THE INVENTION

Methods and polypeptides for targeted activation of antigen binding sites have been reported previously. WO2019086362 and PCT/EP2020/061413 report formation of an anti-CD3 antibody binding site from two precursor proteins by polypeptide chain exchange. Two precursor proteins comprising a destabilized CH3 interface and one of the variable domains of the desired anti-CD3 antibody are described to undergo polypeptide chain exchange and thereby assemble to a protein comprising the desired anti-CD3 antibody.

Cytokines are proteins that modulate the immune response by regulating survival, proliferation, differentiation, and effector functions of leukocytes (Dinarello CA, Eur J Immunol. 2007; 37 (Suppl 1):S34-S45). Cytokines can be classified into families and subfamilies according to structural similarities. The four-α-helix bundle family includes, among others, Interleukin(IL)-2, IL-3, IL-4, IL-5, IL-6, IL-7, IL-9, IL-11, IL-13, IL-15, leukemia inhibitory factor (LIF), ciliary neurotrophic factor (CNTF), cardiotrophin-1 (CT-1), oncostatin M (OSM) and granulocyte-macrophage colony-stimulating factor (GM-CSF) (Nicola N A and Hilton D J, Adv. Protein Chem. 1998; 52:1-65). The IL-12 family comprises heterodimeric cytokines such as IL-12, IL-23, IL-27 and IL-35, of which the α-chains of the respective cytokines belong to the four-α-helix bundle family (Vignali D A A and Kuchroo V J, Nat Immunol. 2012 August; 13(8): 722-728). Homologs of IL-17 are summarized in the IL-17 family (McGeachy M J et al., Immunity. 2019; 50(4):892-906). The IL-1 family consists of IL-1 and IL-18. The cysteine-knot family contains the transforming growth factor beta (TGF-β) cytokines (Sun P D and Davies D R, Annu Rev Biophys Biomol Struct. 1995; 24:269-291).

To date, cytokines have found application for treatment of various conditions, including autoimmunity, viral infections and cancer (Lipiäinen T et al., J Pharm Sci. 2015 February; 104(2):307-26). A wide range of cytokines have been evaluated for the treatment of cancer, including interleukin (IL)-2, IL-12, IL-15, IL-21, and type I interferons (IFN) such as IFN-α (Ardolino M, Hsu J, Raulet D H, Oncotarget 2015 6: 19346-19347). Furthermore, several cytokine products have been approved for clinical use (Lipiäinen T et al., J Pharm Sci. 2015 February; 104(2):307-26).

However, using cytokines alone as therapeutics often entails significant drawbacks. To achieve effective concentrations in tumor tissue, large cytokine quantities need to be administered, which in turn can cause severe adverse effects, including fever, hypotension, fatigue, nausea, anorexia, or neutropenia.

Cytokines have been used in cancer therapy (Waldmann T A, Cold Spring Harb Perspect Biol. 2018 Dec. 3; 10(12)). This problem of systemic distribution can be addressed by fusing the cytokine to a tumor-targeting antibody or antibody-like molecule, enabling a preferential accumulation of the therapeutic agent at the tumor site. Various antibody-cytokine fusions have shown promising results for targeted cancer immunotherapy (Kiefer J D, Neri D, Immunol Rev. 2016; 270(1): 178-192). While antigen-targeted delivery of cytokines can reduce systemic cytokine burden, antigen specificity remains a challenge. Many tumor antigens are merely overexpressed, but not exclusively expressed on tumor cells (Vigneron N, Biomed Res Int. 2015; 2015:948501). On-target off-tumor targeting of can cause severe damage of healthy tissue.

Therefore there is still a need for alternative approaches for cytokine based cancer therapies.

### SUMMARY OF THE INVENTION

The present invention relates to a set of a first and a second precursor protein, wherein each precursor protein comprises two polypeptides that are associated with each other via dimerization domains, wherein at least one of the precursor proteins comprises a moiety selected from a receptor ligand and an enzyme, wherein said moiety is functionally inactive, wherein said moiety is fused to the dimerization domain, wherein upon polypeptide chain exchange between the first and the second precursor protein an activated protein is formed, wherein the activated protein comprises one polypeptide from the first precursor protein and one polypeptides from the second precursor protein, wherein both polypeptides are associated with each other via their dimerization domains, and wherein the activated protein comprises said moiety, characterized in that the activated protein comprises said moiety in functionally active form.

In one embodiment either the first precursor protein or the second precursor protein comprise the moiety selected from a receptor ligand and an enzyme, wherein the moiety is bound to an inactivation moiety. Upon polypeptide chain exchange the inactivation moiety is removed, wherevy the moiety is brought into functionally active form.

In another embodiment the first precursor protein and the second precursor protein comprise complementary subunits of the moiety selected from a receptor ligand and an enzyme. Upon polypeptide chain exchange the resulting activated protein comprises the moiety in functionally active form, i.e. comprising both complementary subunits.

In yet another embodiment the first precursor protein and the second precursor protein comprise complementary parts of an artificially splitted moiety selected from a receptor ligand and an enzyme, wherein one of the complementary parts is inactivated. Upon polypeptide chain exchange the resulting activated protein comprises both parts of the artificially splittet moiety, which are not inactivated, thereby comprising the moiety in functionally active form.

In one embodiment, the the dimerization domains are CH3 domains. In one embodiment the CH3 domains have a modified interface to support polypeptide chain exchance between the first and the second precursor protein.

In one embodiment, the first precursor protein and the second precursor protein specifically bind to an antigen of a target cell. In one embodiment, the first precursor protein and the second precursor protein comprise an antibody fragment specifically binding to an antigen on the surface of a target cell.

In one embodiment, the first precursor protein and the second precursor protein comprise a hinge region. In one embodiment, the first precursor protein and the second precursor protein do not comprise an interchain disulfide bond in the hinge region.

In one embodiment, the activated protein comprises a pair of a VH domains and a VL domain specifically binding to an antigen, wherein the VH domain is comprised in the polypeptide from the first precursor protein and the VL domain is comprised in the polypeptide from the second precursor protein; or the activated protein comprises a pair of a VH domains and a VL domain specifically binding to an antigen, wherein the VL domain is comprised in the polypeptide from the first precursor protein and the VH domain is comprised in the polypeptide from the second precursor protein.

Another aspect of the invention is the set of the invention for therapy.

Another aspect of the invention is the use of a set of a first and a second precursor protein of the invention for the generation of an activated form of said moiety selected from a receptor ligand and an enzyme.

Another aspect of the invention is a therapeutic kit comprising a first and a second precursor protein of the invention.

Another aspect of the invention is a method for providing a therapeutic kit of the invention, comprising the steps of providing recombinantly expressed first precursor protein and recombinantly expressed second precursor protein, and formulating the first and second precursor protein, optionally with a pharmaceutically acceptable carrier to provide the therapeutic kit.

Another aspect of the invention is a protein (activated protein), comprising a functionally active form of a moiety selected from a receptor ligand and an enzyme produced by polypeptide chain exchange between the first precursor protein and the second precursor protein of the invention.

Another aspect of the invention is a method for providing an activated protein comprising a functionally active form of a moiety selected from a receptor ligand and an enzyme, comprising the steps of combining the first precursor polypeptide and the second precursor polypeptide of the invention such that the precursor polypeptides undergo polypeptide chain exchange to form the activated protein.

According to the invention a functionally active receptor ligand or enzyme is formed from two precursor polypeptides by polypeptide chain exchange. Polypeptide chain exchange occurs upon combining the two precursor proteins under appropriate conditions, e.g. when both precursor proteins are in close proximity, like when the are bound on the surface of a target cell. The invention allows targeted activation of therapeutically desired functional moieties at the site of interest and thereby is advantageous for therapy, e.g. for reduced off target toxicity.

### DESCRIPTION OF THE FIGURES

FIG. 1A: Domain arrangement of the antibody core of precursor polypeptides (R1, R2) exemplarily used in the examples. Polypeptide chain exchange leads to formation of product polypeptides (P1 and P2).

FIG. 1B: Domain arrangement of precursor polypeptides described in Example 1 comprising artificially splitted IL-4. Polypeptide chain exchange between precursor proteins (R1, R2) leads to formation of activated product protein P1 and inactive product protein P2.

FIG. 2A: Split design of interleukin-4 (Protein Data Bank 2B8U). 3+1 split of IL-4. One part consisting of the N-terminal helix of interleukin-4 (light gray), the other part consisting of the remaining protein (dark gray).

FIG. 2B: Split design of interleukin-4 (Protein Data Bank 2B8U). 2+2 split of IL-4. Circular permutation generating interleukin-4 DABC. One part consisting of the two N-terminal helices of interleukin-4 DABC (light gray), the other part consisting of the remaining protein (dark gray).

FIG. 3A: SDS-PAGE analysis of the purified precursor proteins (R1, R2) comprising artificially splitted IL-4 and active product (P1) protein according to Example 1. Protein preparations were performed by Kappa Select extraction from cell culture supernatants followed by ion exchange chromatography.

FIG. 3B: Exemplary SEC profile of the purified molecule R2 as described in Example 1. The main peak of the profile represents the protein of interest.

FIG. 4A: IL-4 activity measurements with TF-1 cells. Principle of detecting IL-4 signaling functionality by TF-1 proliferation assay. IL-4 binds to IL-4 receptor on TF-1 cells and induces proliferation.

FIG. 4B: Flow cytometry analysis of antigen expression (Her2, CD38, LeY, CD33) on TF-1 cells.

FIG. 4C: Constructs tested in TF-1 proliferation assay to compare IL-4 and IL-4 DABC activity.

FIG. 4D: TF-1 proliferation assay with molecules depicted in FIG. 4B. IL-4 DABC shows activity similar to IL-4.

FIG. 5A: TF-1 cell proliferation assays with 3+1 split IL-4 precursor proteins with E9Q and R88Q mutations. Molecules targeting CD38, which is expressed on TF-1 cells.

FIG. 5B: TF-1 cell proliferation assays with 3+1 split IL-4 precursor proteins with E9Q and R88Q mutations. Molecules targeting Her2, which is not expressed on TF-1 cells.

FIG. 5C: TF-1 cell proliferation assays with 3+1 split IL-4 precursor proteins with E9Q and R88Q mutations. Comparison of targeted and non targeted polypeptide chain exchange.

FIG. 6A-FIG. 6B: TF-1 cell proliferation assays with split IL-4 precursor proteins and product molecules targeting CD38.

FIG. 7: TF-1 cell proliferation assay with 100 nM 3+1 split IL-4 reactant molecules with different mutations.

FIG. 8A: TF-1 cell proliferation assays with 2+2 split IL-4 precursor proteins with T6D E9A and R81E R88Q mutations. Molecules targeting CD38, which is expressed on TF-1 cells.

FIG. 8B: TF-1 cell proliferation assays with 2+2 split IL-4 precursor proteins with T6D E9A and R81E R88Q mutations. Molecules targeting Her2, which is not expressed on TF-1 cells.

FIG. 8C: TF-1 cell proliferation assays with 2+2 split IL-4 precursor proteins with T6D E9A and R81E R88Q mutations. Direct comparison of targeted and non targeted polypeptide chain exchange.

FIG. 9A: Surface Plasmon Resonance (SPR). SPR setup to study the interaction of IL-4-containing molecules with IL-4 receptor alpha.

FIG. 9B: Surface Plasmon Resonance (SPR). Numerical results of molecules tested by SPR.

FIG. 9C: Surface Plasmon Resonance (SPR). Graphical results of molecules tested by SPR.

FIG. 10A: Design and modular composition of precursor polypeptides used in Example 2 comprising interleukin-2.

FIG. 10B: Three different precursor proteins suitable as reactand R1 in the method shown in FIG. 10B. IL-2v refers to IL-2 variant designed for decreased binding to interleukin-2 receptor alpha; γc referes to the extracellular domain of the common gamma chain; IL-2Rβ refers to the extracellular domain of interleukin-2 receptor beta.

FIG. 11: SDS-PAGE analysis of the purified precursor protein R2, the active product (P1) and three different precursor proteins R1. Protein preparations were performed by Kappa Select extraction from cell culture supernatants followed by ion exchange chromatography.

FIG. 12A: IL-2v activity measurements with CTLL-2 cells. Principle of detecting IL-2v signaling functionality by CTLL-2 proliferation assay. IL-2v binds to IL-2 receptor (consisting of IL-2Rβ and γc) on CTLL-2 cells and induces proliferation.

FIG. 12B: IL-2v activity measurements with CTLL-2 cells. CTLL-2 proliferation assay with described in Example 2.

FIG. 13A: Design and modular composition of precursor polypeptides used in Example 2 comprising interleukin-12. The approach makes use of the p35 and p40 subunits of IL-12. P35i and p40i refer to inactivated versions of p35 and p40. Here, precursor protein R2 carries only an IL-12 p40 subunit but no neighboring IL-12 p35 subunit.

FIG. 13B: Design and modular composition of precursor polypeptides used in Example 2 comprising interleukin-12. Here, precursor protein R2 carries an IL-12 p40 subunit (IL-12 p40) as well as an inactivated IL-12 p35 subunit (IL-12 p35i).

FIG. 14: SDS-PAGE analysis of the purified product P1 and reactant R1 molecules comprising several subunits of IL-12. Protein preparations were performed by Kappa Select extraction from cell culture supernatants followed by ion exchange chromatography. For P1 SDS-PAGE indicated that all polypeptide chains were present in the preparation. For R1 the two heavy chains had a similar molecular weight, hence the two bands overlapped. The presence of both types of heavy chains was, however, confirmed by mass spectrometry.

FIG. 15: IL-12 activity measurements of molecules depicted in FIG. 15 using HEK-Blue IL-12 reporter cells.

FIG. 16: Design and modular composition of precursor polypeptides used in Example 9 comprising NanoBiT® split luciferase enzyme

FIG. 17: Luminescence readout of split luciferase precursor polypeptides R1 and R2 and a combination thereof, at 50 nM targeting CD38 in presence or absence of TF-1 cells expressing CD38 (Example 10).

### DETAILED DESCRIPTION OF THE INVENTION

### 1. Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present disclosure are generally performed according to conventional methods well known in the art. Generally, nomenclatures used in connection with, and techniques of biochemistry, enzymology, molecular, and cellular biology, microbiology, genetics and protein and nucleic acid chemistry and hybridization described herein are those well-known and commonly used in the art.

The terms “a”, “an” and “the” generally include plural referents, unless the context clearly indicates otherwise.

Unless otherwise defined herein the term “comprising of” shall include the term “consisting of”.

The provision of two alternatives using the terms “either . . . or” designates mutually exclusive alternatives, unless the context clearly indicates otherwise.

The term “antigen binding region” as used herein refers to a moiety that specifically binds to a target antigen. The term includes antibodies as well as other natural (e.g. receptors, ligands) or synthetic (e.g. DARPins) molecules capable of specifically binding to a target antigen.

The term “antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The terms “binding site” or “antigen-binding site” as used herein denotes the region or regions of an antigen binding region to which the antigen actually binds. In case the antigen binding region is an antibody, the antigen-binding site includes antibody heavy chain variable domains (VH) and/or antibody light chain variable domains (VL), or pairs of VH/VL. Antigen-binding sites derived from antibodies that specifically bind to a target antigen can be derived a) from known antibodies specifically binding to the antigen or b) from new antibodies or antibody fragments obtained by de novo immunization methods using inter alia either the antigen protein or nucleic acid or fragments thereof or by phage display methods.

When being derived from an antibody, an antigen-binding site of an antibody according to the invention can contain six complementarity determining regions (CDRs) which contribute in varying degrees to the affinity of the binding site for antigen. There are three heavy chain variable domain CDRs (CDRH1, CDRH2 and CDRH3) and three light chain variable domain CDRs (CDRL1, CDRL2 and CDRL3). The extent of CDR and framework regions (FRs) is determined by comparison to a compiled database of amino acid sequences in which those regions have been defined according to variability among the sequences. Also included within the scope of the invention are functional antigen binding sites comprised of fewer CDRs (i.e., where binding specificity is determined by three, four or five CDRs). For example, less than a complete set of 6 CDRs may be sufficient for binding.

The term “valent” as used herein denotes the presence of a specified number of binding sites in an antibody molecule. A natural antibody for example has two binding sites and is bivalent. As such, the term “trivalent” denotes the presence of three binding sites in an antibody molecule.

An “antibody fragment” refers to a molecule other than an intact antibody that comprises a portion of an intact antibody that binds the antigen to which the intact antibody binds. Examples of antibody fragments include but are not limited to Fv, Fab, Fab, Fab-SH, F(ab)2; diabodies; linear antibodies; single-chain antibody molecules (e.g. scFv, scFab); and multispecific antibodies formed from antibody fragments.

“Specificity” refers to selective recognition of a particular epitope of an antigen by the antigen binding region, e.g. an antibody. Natural antibodies, for example, are monospecific. The term “monospecific antibody” as used herein denotes an antibody that has one or more binding sites each of which bind to the same epitope of the same antigen. “Multispecific antibodies” bind two or more different epitopes (for example, two, three, four, or more different epitopes). The epitopes may be on the same or different antigens. An example of a multispecific antibody is a “bispecific antibody” which binds two different epitopes. When an antibody possesses more than one specificity, the recognized epitopes may be associated with a single antigen or with more than one antigen.

An epitope is a region of an antigen that is bound by an antigen binding region, e.g. an antibody. The term “epitope” includes any polypeptide determinant capable of specific binding to an antibody or antigen binding region. In certain embodiments, epitope determinants include chemically active surface groupings of molecules such as amino acids, glycan side chains, phosphoryl, or sulfonyl, and, in certain embodiments, may have specific three dimensional structural characteristics, and/or specific charge characteristics.

As used herein, the terms “binding” and “specific binding” refer to the binding of the antibody or antigen binding region to an epitope of the antigen in an in vitro assay, preferably in a plasmon resonance assay (BIAcore®, GE-Healthcare Uppsala, Sweden) with purified wild-type antigen. In certain embodiments, an antibody or antigen binding region is said to specifically bind an antigen when it preferentially recognizes its target antigen in a complex mixture of proteins and/or macromolecules.

The affinity of the binding of an antibody to an antigen is defined by the terms kₐ (rate constant for the association of the antibody from the antibody/antigen complex), k_{d} (dissociation constant), and K_{D} (k_{d}/kₐ). In one embodiment binding or that/which specifically binds to means a binding affinity (K_{D}) of 10⁻⁸ mol/l or less, in one embodiment 10⁻⁸ M to 10⁻¹³ mol/l. Thus, an antigen binding region, particularly an antibody binding site, specifically binds to each antigen for which it is specific with a binding affinity (K_{D}) of 10⁻⁸ mol/l or less, e.g. with a binding affinity (K_{D}) of 10⁻⁸ to 10⁻¹³ mol/l. in one embodiment with a binding affinity (K_{D}) of 10⁻⁹ to 10⁻¹³ mol/l.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “constant domains” or “constant region” as used within the current application denotes the sum of the domains of an antibody other than the variable region. The constant region is not directly involved in binding of an antigen, but exhibits various effector functions.

Depending on the amino acid sequence of the constant region of their heavy chains, antibodies are divided in the “classes”: IgA, IgD, IgE, IgG and IgM, and several of these may are further divided into subclasses, such as IgG1, IgG2, IgG3, and IgG4, IgA1 and IgA2. The heavy chain constant regions that correspond to the different classes of antibodies are called α, δ, ε, γ and μ, respectively. The light chain constant regions (CL) which can be found in all five antibody classes are called κ (kappa) and λ (lambda).

The “constant domains” as used herein are, preferably, from human origin, which is from a constant heavy chain region of a human antibody of the subclass IgG1, IgG2, IgG3, or IgG4 and/or a constant light chain kappa or lambda region. Such constant domains and regions are well known in the state of the art and e.g. described by Kabat, et al., Sequences of Proteins of Immunological Interest, 5th ed., Public Health Service, National Institutes of Health, Bethesda, MD (1991).

In wild type antibodies, the “hinge region” is a flexible amino acid stretch in the central part of the heavy chains of the IgG and IgA immunoglobulin classes, which links the two heavy chains by disulfide bonds, i.e. “interchain disulfide bonds” as they are formed between the two heavy chains. The hinge region of human IgG1 is generally defined as stretching from about Glu216, or about Cys226, to about Pro230 of human IgG1 (Burton, Molec. Immunol. 22:161-206 (1985)). By deleting cysteine residues in the hinge region or by substituting cysteine residues in the hinge region by other amino acids, such as serine, disulfide bond formation in the hinge region is avoided.

The “light chains” of antibodies from any vertebrate species can be assigned to one of two distinct types, called kappa (κ) and lambda (λ), based on the amino acid sequences of their constant domains. A wild type light chain typically contains two immunoglobulin domains, usually one variable domain (VL) that is important for binding to an antigen and a constant domain (CL).

Several different types of “heavy chains” exist that define the class or isotype of an antibody. A wild type heavy chain contains a series of immunoglobulin domains, usually with one variable domain (VH) that is important for binding antigen and several constant domains (CH1, CH2, CH3, etc.).

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The “CH2 domain” of a human IgG Fc region usually extends from an amino acid residue at about position 231 to an amino acid residue at about position 340. The multispecific antibody is devoid of a CH2 domain. By “devoid of a CH2 domain” is meant that the antibodies according to the invention do not comprise a CH2 domain.

The “CH3 domain” comprises the stretch of residues C-terminal to a CH2 domain in an Fc region (i.e. from an amino acid residue at about position 341 to an amino acid residue at about position 447 of an IgG). The “CH3 domains” herein are variant CH3 domains, wherein the amino acid sequence of the natural CH3 domain was subjected to at least one distinct amino acid substitution (i.e. modification of the amino acid sequence of the CH3 domain) in order to promote heterodimerization of the two CH3 domains facing each other within the multispecific antibody.

Typically, in the heterodimerization approaches known in the art, the CH3 domain of one heavy chain and the CH3 domain of the other heavy chain are both engineered in a complementary manner so that the heavy chain comprising one engineered CH3 domain can no longer homodimerize with another heavy chain of the same structure. Thereby the heavy chain comprising one engineered CH3 domain is forced to heterodimerize with the other heavy chain comprising the CH3 domain, which is engineered in a complementary manner.

One heterodimerization approach known in the art is the so-called “knobs-into-holes” technology, which is described in detail providing several examples in e.g. WO 96/027011, Ridgway, J. B., et al., Protein Eng. 9 (1996) 617-621; Merchant, A. M., et al., Nat. Biotechnol. 16 (1998) 677-681; and WO 98/050431, which are herein included by reference. In the “knobs-into-holes” technology, within the interface formed between two CH3 domains in the tertiary structure of the antibody, particular amino acids on each CH3 domain are engineered to produce a protuberance (“knob”) in one of the CH3 domains and a cavity (“hole”) in the other one of the CH3 domains, respectively. In the tertiary structure of the multispecific antibody the introduced protuberance in the one CH3 domain is positionable in the introduced cavity in the other CH3 domain.

In combination with the substitutions according to the knobs-into-holes technology, additional interchain disulfide bonds may be introduced into the CH3 domains to further stabilize the heterodimerized polypeptides (Merchant, A. M., et al., Nature Biotech. 16 (1998) 677-681). Such interchain disulfide bonds are formed, e.g. by introducing the following amino acid substitutions into the CH3 domains: D399C in one CH3 domain and K392C in the other CH3 domain; Y349C in one CH3 domain and S354C in the other CH3 domain; Y349C in one CH3 domain and E356C in the other CH3 domain; Y349C in one CH3 domain and E357C in the other CH3 domain; L351C in one CH3 domain and S354C in the other CH3 domain; T394C in one CH3 domain and V397C in the other CH3 domain. A “cysteine mutation” as used herein refers to one amino acid substitution of an amino acid in a CH3 domain by cysteine that is capable of forming an interchain disulfide bond with another, matching, amino acid substitution of an amino acid in a second CH3 domain by cysteine.

The term “pharmaceutical composition” refers to a preparation which is in such form as to permit the biological activity of an active ingredient contained therein to be effective, and which contains no additional components which are unacceptably toxic to a subject to which the composition would be administered. A pharmaceutical composition of the present invention can be administered by a variety of methods known in the art. As will be appreciated by the skilled artisan, the route and/or mode of administration will vary depending upon the desired results. To administer an antibody according to the invention by certain routes of administration, it may be necessary to coat the antibody with, or co-administer the antibody with, a material to prevent its inactivation. For example, the heterodimeric polypeptide may be administered to a subject in an appropriate carrier, for example, liposomes, or a diluent. Pharmaceutically acceptable diluents include saline and aqueous buffer solutions.

A pharmaceutical composition comprises an effective amount of the heterodimeric polypeptides provided with the invention. An “effective amount” of an agent, e.g., a heterodimeric polypeptide, refers to an amount effective, at dosages and for periods of time necessary, to achieve the desired therapeutic or prophylactic result. In particular, the “effective amount” denotes an amount of a heterodimeric polypeptide of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the heterodimeric polypeptide molecules used, disease state being treated, the severity or the disease treated, the age and relative health of the subject, the route and form of administration, the judgment of the attending medical or veterinary practitioner, and other factors.

A “pharmaceutically acceptable carrier” refers to an ingredient in a pharmaceutical formulation, other than an active ingredient, which is nontoxic to a subject. Pharmaceutically acceptable carriers include any and all solvents, dispersion media, coatings, antibacterial and antifungal agents, isotonic and absorption delaying agents, and the like that are physiologically compatible. In one preferred embodiment, the carrier is suitable for intravenous, intramuscular, subcutaneous, parenteral, spinal or epidermal administration (e.g. by injection or infusion).

The pharmaceutical compositions according to the invention may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991). In particular, for variable domains and for the light chain constant domain CL of kappa and lambda isotype, the Kabat numbering system (see pages 647-660) of Kabat, et al., Sequences of Proteins of Immunological Interest, 5th ed., Public Health Service, National Institutes of Health, Bethesda, MD (1991) is used and for the constant heavy chain domains (CH1, Hinge, CH2 and CH3) the Kabat EU index numbering system (see pages 661-723) is used.

Amino acid “substitutions” or “replacements” or “mutations” (all terms are herein used interchangeably) within the polypeptide chains are prepared by introducing appropriate nucleotide changes into the antibody DNA, or by nucleotide synthesis. Such modifications can be performed, however, only in a very limited range. For example, the modifications do not alter the above mentioned antibody characteristics such as the IgG isotype and antigen binding, but may further improve the yield of the recombinant production, protein stability or facilitate the purification. In certain embodiments, antibody variants having one or more conservative amino acid substitutions are provided. A “double mutation” as referred herein means that both of the indicated amino acid substitutions are present in the respective polypeptide chain.

The term “amino acid” as used herein denotes an organic molecule possessing an amino moiety located at a-position to a carboxylic group. Examples of amino acids include: arginine, glycine, ornithine, lysine, histidine, glutamic acid, asparagic acid, isoleucine, leucine, alanine, phenylalanine, tyrosine, tryptophane, methionine, serine, proline. The amino acid employed is optionally in each case the L-form. The term “positively charged” or “negatively charged” amino acid refers to the amino acid side-chain charge at pH 7.4. Amino acids may be grouped according to common side-chain properties:
(1) hydrophobic: Norleucine, Met, Ala, Val, Leu, Ile, Trp, Tyr, Phe;
(2) neutral hydrophilic: Cys, Ser, Thr, Asn, Gln;
(3) acidic or negatively charged: Asp, Glu;
(4) basic or positively charged: His, Lys, Arg;
(5) residues that influence chain orientation: Gly, Pro.

TABLE
Amino acids with specific properties

| | 3- | 1- | Side-chain | Side-chain |
|---|---|---|---|---|
| Amino Acid | Letter | Letter | polarity | charge (pH 7.4) |
| Alanine | Ala | A | nonpolar | neutral |
| Arginine | Arg | R | basic polar | positive |
| Asparagine | Asn | N | polar | neutral |
| Aspartic acid | Asp | D | acidic polar | negative |
| Cysteine | Cys | C | nonpolar | neutral |
| Glutamic acid | Glu | E | acidic polar | negative |
| Glutamine | Gln | Q | polar | neutral |
| Glycine | Gly | G | nonpolar | neutral |
| Histidine | His | H | basic polar | positive (10%) |
| | | | | neutral (90%) |
| Isoleucine | Ile | I | nonpolar | neutral |
| Leucine | Leu | L | nonpolar | neutral |
| Lysine | Lys | K | basic polar | positive |
| Methionine | Met | M | nonpolar | neutral |
| Phenylalanine | Phe | F | nonpolar | neutral |
| Proline | Pro | P | nonpolar | neutral |
| Serine | Ser | S | polar | neutral |
| Threonine | Thr | T | polar | neutral |
| Tryptophan | Trp | W | nonpolar | neutral |
| Tyrosine | Tyr | Y | polar | neutral |
| Valine | Val | V | nonpolar | neutral |

A “polypeptide chain exchange” between two proteins occurs, when two polypeptide chains from a first precursor protein dissociate and two polypeptide chains from a second precursor protein dissociate and a polypeptide chain derived from the first precursor protein pairs with a polypeptide chain derived from the second precursor protein. In consequence, a “product” protein is formed comprising a polypeptide chain from the first precursor polypeptide and a polypeptide chain from the second precursor polypeptide. Both polypeptide chains are associated via their dimerization domains in the product protein.

A precursor protein of the invention comprises a “moiety selected from a receptor ligand and an enzyme”. The term “moiety” as used herein refers to protein selected from a receptor ligand or an enzyme; a fragment thereof; or a substitution variant of the receptor ligand, the enzyme or the fragment thereof. Exemplary receptor ligands are cytokines. Typically, receptor ligands and enzymes are protein complexes made up of more than one subunit. Hence, a “subunit” as referred to herein is a a single polypeptide molecule that assembles with other polypeptides (“subunits”) to form a protein complex.

The moiety consequently has a biological function, namely the biological function of the receptor ligand (i.e. formation a complex between the ligand and the receptor) or the enzyme (i.e. biocatalytic activity). By the term “functionally active” as used herein is meant that said moiety exhibits its biological function under physiological conditions. By the term “functionally inactive” as used herein is meant that said the activity of the moiety is reduced to less than 5% of the activity of the corresponding functionally active full length receptor ligand or enzyme. Preferably, the “functionally inactive” moiety has no biological activity.

The moiety is comprised in a polypeptide chain of the precursor protein that has a dimerization domain, preferably via a peptide linker. Typically, the peptide connectors are composed of flexible amino acid residues like glycine and serine. Thus, typical peptide connectors used for fusing the moiety to polypeptides are glycine-serine linkers, i.e. peptide connectors consisting of a pattern of glycine and serine residues. Depending on the structure of the moiety, the precursor protein comprises one or two fragments of said moiety such that the moiety is functionally inactive, or the precursor protein comprises said moiety and an inactivation moiety that is bount to said moiety such that the moiety is functionally inactive.

### 2. Embodiments of the Invention

In a first aspect the invention relates to a set of a first and a second precursor protein, wherein each precursor protein comprises two polypeptides that are associated with each other via dimerization domains, wherein at least one of the precursor proteins comprises a moiety selected from a receptor ligand and an enzyme, wherein said moiety is functionally inactive, wherein said moiety is fused to the dimerization domain, wherein upon polypeptide chain exchange between the first and the second precursor protein an activated protein is formed, wherein the activated protein comprises one polypeptide from the first precursor protein and one polypeptides from the second precursor protein, wherein both polypeptides are associated with each other via their dimerization domains, and wherein the activated protein comprises said moiety, characterized in that the activated protein comprises said moiety in functionally active form.

### A) First Alternative: Bound to Inactivation Moiety

In a first embodiment of the first aspect the invention relates to a set of a first and a second precursor protein according to the invention, wherein either the first precursor protein or the second precursor protein comprise the moiety selected from a receptor ligand and an enzyme, wherein the moiety is bound to an inactivation moiety. In one embodiment the moiety is a receptor ligand and the inactivation moiety is the corresponding receptor, a ligand-binding subunit thereof or another protein that inactivates the moiety. In one embodiment the receptor ligand is a cytokine and the inactivation moiety is the corresponding cytokine receptor or a cytokine-binding subunit thereof. In one embodiment the receptor ligand is IL-2v and the inactivation moiety is selected from a subunit of IL-2R, preferably IL-2Rbeta, IL-2Rgamma-chain, IL-2Rbeta_gamma-chain. The “corresponding receptor” as referred to herein is the receptor that is bound by the receptor ligand comprised in the precursor protein in order to exhibit the biological function of the receptor ligand. For example, if the receptor ligand is a cytokine, e.g. IL-2, the corresponding receptor is the cytokine receptor of said cytokine, e.g. IL-2R. A “ligand-binding subunit” of the corresponding receptor refers to a subunit of said receptor involved in binding of said receptor ligand to said receptor. For example, if the receptor ligand is IL-2, the corresponding receptor is IL-2R and a ligand-binding subunit is IL-2Rbeta.

### B) Second Alternative: Several Subunits

In a second embodiment of the first aspect the invention relates to a set of a first and a second precursor protein according to the invention, wherein the first precursor protein and the second precursor protein comprise complementary subunits of the moiety selected from a receptor ligand and an enzyme. In one embodiment the first precursor protein comprises a first unmodified subunit of the moiety and a second subunit of the moiety, wherein the second subunit comprises an inactivating mutation; and wherein the second precursor protein comprises the second unmodified subunit of the moiety. The term “unmodified subunit” refers to a protein subunit of the moiety that does not comprise any mutations that abolish their biological function. In one embodiment the unmodified subunit has an amino acid sequence identical to the amino acid sequence of the natural respective subunit of the moiety. The term “inactivating mutation” refers to an addition, substitution or deletion of an amino acid in the amino acid sequence of the natural subunit of said moiety. A full length moiety comprising the subunit having the inactivating mutation is functionally inactive.

In one embodiment the moiety is a receptor ligand. In one embodiment the receptor ligand is a cytokine, wherein the first precursor protein comprises a first subunit of the cytokine and a second subunit of the cytokine, wherein the second subunit comprises an inactivating mutation; and wherein the second precursor protein comprises the second unmodified subunit of the cytokine. In one embodiment the first precursor protein comprises a IL-12 p35 and IL-12 p40 comprising an inactivating mutation; and wherein the second precursor protein comprises the unmodified IL-12 p40. In one embodiment the first precursor protein comprises a IL-12 p35 and IL-12 p40 comprising an inactivating mutation; and wherein the second precursor protein comprises the unmodified IL-12 p40 and IL-12 p35 comprising an inactivating mutation.

### C) Third Alternative: Splitted Receptor Ligand or Enzyme

In a third embodiment of the first aspect the invention relates to a set of a first and a second precursor protein according to the invention, wherein the first precursor protein and the second precursor protein comprise complementary parts of an artificially splitted moiety selected from a receptor ligand or an enzyme, wherein one of the complementary parts is inactivated. The term “artificially splitted moiety” refers to a functionally active protein moiety that is split into two or more (preferably two) fragments, herein termed “split fragments”. Each split fragment is inactive with respect to the function of the functionally active moiety. When all split fragments are associated, functionality of the functionally active moiety is restored. The precursor protein is arranged such that it comprises one functional (e.g. unmutated) and one inactivated (e.g. mutated) part of the artificially splitted moiety. The other precursor protein is arranged such that upon polypeptide chain exchange between the two precursor proteins the two complementary parts of the artificially splittet moiety that result in formation of a functionally active moiety are comprised in the activated protein.

In one embodiment the moiety is a receptor ligand. In one embodiment the the receptor ligand is a cytokine. In one embodiment the artificially splitted moiety is a split cytokine. “Split cytokines” have been described in the art, e.g. Venetz et al. J Biol Chem. 2016 Aug. 26; 291(35): 18139-18147.

In one embodiment the receptor ligand is an enzyme. In one embodiment the the artificially splitted moiety is a split enzyme. “Split enzyme” have been described in the art, e.g. Littmann et al. Scientific Reports volume 8, Article number: 17179 (2018).

### D) Precursor Proteins

Precursor proteins comprised in a set of the invention are capable of undergoing polypeptide chain exchange. A general domain arrangement of such pairs of precursor proteins has been described before, e.g. WO2019/077092, WO2019086362, PCT/EP2020/061412 and PCT/EP2020/061413, that are incorporated by reference herein. In certain embodiments, each precursor protein is of a half-antibody shape, namely comprising one antigen binding site (preferably a Fab fragment) arranged via a hinge region to one dimerizing Fc based region. Such precursor proteins have been described, e.g. WO2019/077092, WO2019086362, PCT/EP2020/061412 and PCT/EP2020/061413. Said dimerizing Fc based region comprises a pair of CH3 domains, optionally further comprising a pair of CH2 domains arranged at the N-terminus of said CH3 domains (thus, two dimerizing polypeptides of the precursor protein comprise a domain arrangement of CH2-CH3 from N- to C-terminus) or a pair of VH and VL domains arranged at the N-terminus of said CH3 domains (thus, one polypeptide of the precursor protein comprises a domain arrangement of VL-CH3 from N- to C-terminus and the other polypeptide of the precursor protein comprises a domain arrangement of VH-CH3 from N- to C-terminus). The pair of VH and VL domains may also be arranged at the N-terminus of CH2 domains, that are fused to the N-terminus of the CH domains (thus, one polypeptide of the precursor protein comprises a domain arrangement of VL-CH2-CH3 from N- to C-terminus and the other polypeptide of the precursor protein comprises a domain arrangement of VH-CH2-CH3 from N- to C-terminus). In order to support assembly of the precursor proteins, including dimerization the two polypeptides comprising a dimerization domain and yet allowing polypeptide chain exchange with another, different, precursor protein, the dimerization domains comprised in the precursor proteins are modified.

a. Dimerization Domains

In one embodiment of the invention, the dimerization domains are CH3 domains. The “CH3 domain” comprises the stretch of residues C-terminal to a CH2 domain in an Fc region (i.e. from an amino acid residue at about position 341 to an amino acid residue at about position 447 of an IgG). The “CH3 domains” herein are variant CH3 domains, wherein the amino acid sequence of the natural CH3 domain was subjected to at least one distinct amino acid substitution (i.e. modification of the amino acid sequence of the CH3 domain) in order to promote heterodimerization of the two CH3 domains facing each other within the precursor protein.

In one embodiment, each precursor protein comprises CH3 domains having knob-into-hole modifications, cysteine mutations and destabilizing mutations as defined above.

CH3 domains may be of any IgG isotype, however the CH3 domains of the two precursor proteins are of the same IgG isotype. In one embodiment the CH3 domains are of IgG1 isotype. In one embodiment the CH3 domains are of IgG3 isotype.

### Knobs-Into-Holes Mutations

In one embodiment the first precursor protein and the second precursor protein each comprise two polypeptides comprising a CH3 domain, wherein one CH3 domains comprises a knob mutation and the other CH3 domain comprises a hole mutation. According to this embodiment, the CH3 domains comprised in a precursor protein comprise knob-into-hole modification. The “knob-into-holes” technology is well known in the art and is described in detail providing several examples in e.g. WO 96/027011, Ridgway, J. B., et al., Protein Eng. 9 (1996) 617-621; Merchant, A. M., et al., Nat. Biotechnol. 16 (1998) 677-681; and WO 98/050431, which are herein included by reference. In the “knobs-into-holes” technology, within the interface formed between two CH3 domains in the tertiary structure of the antibody, particular amino acids on each CH3 domain are engineered to produce a protuberance (“knob”) in one of the CH3 domains and a cavity (“hole”) in the other one of the CH3 domains, respectively. In the tertiary structure of the multispecific antibody the introduced protuberance in the one CH3 domain is positionable in the introduced cavity in the other CH3 domain.

In one embodiment the knob mutation comprised in the first precursor protein is identical to the knob mutation comprised in the second precursor protein. In one embodiment the knob mutation is T366W. In one embodiment the hole mutation is T366S L368A Y407V.

Further techniques, apart from the “knobs-into-holes” technology as mentioned before, for modifying the CH3 domains in order to enforce heterodimerization are known in the art. These technologies, especially the ones described in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291 are contemplated herein as alternatives to the “knobs-into-holes technology” for the precursor proteins provided by the invention. All those technologies involve engineering of CH3 domains in a complementary manner, by introduction of amino acids of opposite charge or different side chain volume, thereby supporting heterodimerization.

### Cysteine Mutations

In combination with the substitutions according to the knobs-into-holes technology, additional interchain disulfide bonds may be introduced into the CH3 domains to further stabilize the heterodimerized polypeptides (Merchant, A. M., et al., Nature Biotech. 16 (1998) 677-681). Such interchain disulfide bonds are formed, e.g. by introducing the following amino acid substitutions into the CH3 domains: D399C in one CH3 domain and K392C in the other CH3 domain; Y349C in one CH3 domain and S354C in the other CH3 domain; Y349C in one CH3 domain and E356C in the other CH3 domain; Y349C in one CH3 domain and E357C in the other CH3 domain; L351C in one CH3 domain and S354C in the other CH3 domain; T394C in one CH3 domain and V397C in the other CH3 domain. In one embodiment either i) the CH3 domain comprising the knob mutation of the first precursor protein comprises a cysteine mutation and the CH3 domain comprising the hole mutation of the second precursor protein comprises a cysteine mutation, or ii) wherein the CH3 domain comprising the hole mutation of the first precursor protein comprises a cysteine mutation and the CH3 domain comprising the knob mutation of the second precursor protein comprises a cysteine mutation. A “cysteine mutation” as used herein refers to one amino acid substitution of an amino acid in a CH3 domain by cysteine that is capable of forming an interchain disulfide bond with another, matching, amino acid substitution of an amino acid in a second CH3 domain by cysteine.

In one embodiment of the invention, the CH3 domains of the precursor proteins comprise a second pattern of mutations, i.e. substitutions of distinct amino acids in the CH3/CH3 interface by cysteine in order to allow formation of interchain disulfide bonds between two CH3 domains having cysteine substitutions at interacting positions.

Thus, in one embodiment of the invention either i) the CH3 domain comprising the knob mutation of the first precursor protein comprises a cysteine mutation and the CH3 domain comprising the hole mutation of the second precursor protein comprises a cysteine mutation, or ii) the CH3 domain comprising the hole mutation of the first precursor protein comprises a cysteine mutation and the CH3 domain comprising the knob mutation of the second precursor protein comprises a cysteine mutation. In other words in one embodiment, either i) within the first heterodimeric polypeptide the CH3 domain comprising the knob mutation comprises a cysteine mutation and the CH3 domain comprising the hole mutation does not comprise a cysteine mutation and within the second heterodimeric polypeptide the CH3 domain comprising the knob mutation does not comprise a cysteine mutation and the CH3 domain comprising the hole mutation comprises a cysteine mutation, or ii) within the first heterodimeric polypeptide the CH3 domain comprising the knob mutation does not comprise a cysteine mutation and the CH3 domain comprising the hole mutation comprises a cysteine mutation and within the second heterodimeric polypeptide the CH3 domain comprising the knob mutation comprises a cysteine mutation and the CH3 domain comprising the hole mutation does not comprise a cysteine mutation.

In one embodiment, either i) the CH3 domain comprising the knob mutation of the first precursor protein comprises a first cysteine mutation and the CH3 domain comprising the hole mutation of the second precursor protein comprises a second cysteine mutation, or ii) the CH3 domain comprising the hole mutation of the first precursor protein comprises a first cysteine mutation and the CH3 domain comprising the knob mutation of the second precursor protein comprises a second cysteine mutation, wherein the first and second cysteine mutations are selected from the following pairs:

| First cysteine mutation | Second cysteine mutation |
|---|---|
| D399C | K392C |
| Y349C | S354C |
| Y349C | E356C |
| Y349C | E357C |
| L351C | S354C |
| T394C | V397C |

In one embodiment the first cysteine mutation is Y349C and the second cysteine mutation is S354C.

In one embodiment of the invention i) the CH3 domain comprising the knob mutation of the first precursor protein comprises a substitution S354C and the CH3 domain comprising the hole mutation of the second precursor protein comprises a substitution Y349C, or ii) the CH3 domain comprising the hole mutation of the first precursor protein comprises a substitution Y349C and the CH3 domain comprising the knob mutation of the second precursor protein comprises a substitution S354C.

In one embodiment of the invention, within the first precursor protein the CH3 domain comprising the knob mutation comprises a substitution S354C and the CH3 domain comprising the hole mutation comprises Y at position 349; and wherein within the second precursor protein the CH3 domain comprising the hole mutation comprises a substitution Y349C and the CH3 domain comprising the knob mutation comprises S at position 354.

In one embodiment of the invention i) the CH3 domain comprising the knob mutation of the first precursor protein comprises substitutions T366W S354C and the CH3 domain comprising the hole mutation of the second precursor protein comprises substitutions T366S L368A Y407V Y349C, or ii) the CH3 domain comprising the hole mutation of the first precursor protein comprises substitutions T366S L368A Y407V Y349C and the CH3 domain comprising the knob mutation of the second precursor protein comprises substitutions T366W S354C.

In one embodiment of the invention, within the first precursor protein the CH3 domain comprising the knob mutation comprises a substitution T366W S354C and the CH3 domain comprising the hole mutation comprises Y at position 349 and substitutions T366S L368A Y407V; and wherein within the second precursor protein the CH3 domain comprising the hole mutation comprises substitutions T366S L368A Y407V Y349C and the CH3 domain comprising the knob mutation comprises S at position 354 and a substitution T366W.

In one embodiment of the invention, the CH3 domains of the precursor proteins do not comprise an interchain disulfide bond.

### Destabilizing Mutations

In one embodiment the CH3 domains have a modified interface to support polypeptide chain exchance between the first and the second precursor protein. Precursor proteins of the invention comprise in only one of their CH3 domains an amino acid substitution “destabilizing the CH3/CH3 interface”, also referred to herein as “destabilizing mutations”. With these termini, amino acid substitutions are meant that are arranged in only one of the CH3 domains that are associated in the heterodimeric precursor protein. In said CH3 domain, one or more amino acid positions known to interact within the CH3/CH3 interface, e.g. as disclosed in the prior art related to CH3-heterodimerization strategies indicated above, is replaced by an amino acid with another site-chain property. In contrast to heterodimerization strategies, wherein typically a pair of interacting amino acids in the associated CH3 domains is substituted (i.e. one or more amino acid residues in one CH3 domain involved in the heterodimer; and one or more amino acid residues in the other CH3 domain involved in the heterodimer), the destabilizing mutation is arranged in only one of the CH3 domains involved in the precursor proteins according to the invention. Exemplary amino acid substitutions destabilizing the CH3/CH3 interface are listed below. All exemplary amino acid substitutions specifically disclosed herein are arranged such that the substituted amino acids interact in the CH3/CH3 interface within a pair of said CH3 domains.

In one embodiment either i) the CH3 domain of the first precursor protein comprising the knob mutation and the CH3 domain of the second precursor protein comprising the hole mutation, or ii) the CH3 domain of the first precursor protein comprising the hole mutation and the CH3 domain of the second precursor protein comprising the knob mutation comprise at least one complementary destabilizing mutation, whereas the other two CH3 domains of the first and the second precursor polypeptide do not comprise a destabilizing mutation.

According to the invention, either i) the CH3 domain of the first precursor protein comprising the knob mutation and the CH3 domain of the second precursor protein comprising the hole mutation, or ii) the CH3 domain of the first precursor protein comprising the hole mutation and the CH3 domain of the second precursor protein comprising the knob mutation comprise one or more destabilizing mutations. The one or more destabilizing mutations within the first and second precursor protein are selected such that they interact in the CH3/CH3 interface of the product polypeptide formed by polypeptide chain exchange between the precursor polypeptides.

In case the CH3 domain comprising a knob mutation of a precursor protein comprises a destabilizing mutation, the CH3 domain comprising the hole mutation of said precursor protein does not comprise a destabilizing mutation. When a CH3 domain “does not comprise a destabilizing mutation” it comprises the wild type amino acid residue at the position interacting in a wild type immunoglobulin CH3 domain of the same class with the amino acid residue at the position of the destabilizing mutation comprised in the corresponding CH3 domain.

### 1^{st} Set of Mutations (FORCE/PACE1.0 Mutations)

A first set of destabilizing mutations has been disclosed in WO2019/077092, and WO2019086362.

In one embodiment of the invention, the CH3 domain with the hole mutation comprises at least one amino acid substitution, i.e. destabilizing mutation, selected from the group of E345R, Q347K, Y349W, Y349E, L351F, L351Y, S354E, S354V, D356S, D356A, D356K, E357S, E357A, E357L, E357F, E357K, K360S, K360E, Q362E, S364V, S364L, T366I, L368F, L368V, K370E, N390E, K392E, K392D, T394I, V397Y, D399A, D399K, S400K, D401R, F405W, Y407W, Y407L, Y407I, K409D, K409E, K409I, K439E, L441Y, C349Y, S366T, A368L, V407Y, C354S, and W366T; and the CH3 domain with the knob mutation comprises at least one amino acid substitution, i.e. destabilizing mutation, selected from the group of E345R, Q347K, Y349W, Y349E, L351F, L351Y, S354E, S354V, D356S, D356A, D356K, E357S, E357A, E357L, E357F, E357K, K360S, K360E, Q362E, S364V, S364L, T366I, L368F, L368V, K370E, N390E, K392E, K392D, T394I, V397Y, D399A, D399K, S400K, D401R, F405W, Y407W, Y407L, Y407I, K409D, K409E, K409I, K439E, L441Y, C349Y, S366T, A368L, V407Y, C354S, and W366T.

In one embodiment, the CH3 domain with the hole mutation comprises at least one amino acid substitution, i.e. destabilizing mutation, at position 357 or 356; and the CH3 domain with the knob mutation comprises at least one amino acid substitution, i.e. destabilizing mutation, at position 370 or 439. In one embodiment, the CH3 domain with the hole mutation comprises at least one amino acid substitution, i.e. destabilizing mutation, at position 356; and the CH3 domain with the knob mutation comprises at least one amino acid substitution, i.e. destabilizing mutation, at position 439. In one embodiment, the CH3 domain with the hole mutation comprises at least one amino acid substitution, i.e. destabilizing mutation, at position 357; and the CH3 domain with the knob mutation comprises at least one amino acid substitution, i.e. destabilizing mutation, at position 370.

In one embodiment the CH3 domain with the hole mutation of one (e.g. first) precursor protein comprises a D356K mutation, and the CH3 domain with the knob mutation of the other (e.g. second) precursor protein comprises a K439E mutation. In one embodiment the CH3 domain with the hole mutation of one (e.g. first) precursor protein comprises a E357K mutation, and the CH3 domain with the knob mutation of the other (e.g. second) precursor protein comprises a K370E mutation.

### 2^{nd} Set of Mutations (FORCE2.0 Mutations)

A second set of destabilizing mutations has been disclosed in PCT/EP2020/061412.

In one embodiment of the invention, the CH3 domain with the hole mutation comprises at least one amino acid substitution, i.e. destabilizing mutation, selected from the group of replacement of S354 with a hydrophobic amino acid; replacement of D356 with a positively charged amino acid; replacement of E357 with a positively charged amino acid or with a hydrophobic amino acid; replacement of D356 with a positively charged amino acid, and replacement of E357 with a positively charged amino acid or with a hydrophobic amino acid; replacement of S364 with a hydrophobic amino acid; replacement of A368 with a hydrophobic amino acid; replacement of E392 with a negatively charged amino acid; replacement of T394 with a hydrophobic amino acid; replacement of D399 with a hydrophobic amino acid and replacement of S400 with a positively charged amino acid; replacement of D399 with a hydrophobic amino acid and replacement of F405 with a positively charged amino acid; replacement of V407 with a hydrophobic amino acid; and replacement of K409 with a negatively charged amino acid; and replacement of K439 with a negatively charged amino acid; and the CH3 domain with the knob mutation comprises at least one amino acid substitution, i.e. destabilizing mutation, selected from the group of replacement of Q347 with a positively charged amino acid, and replacement of K360 with a negatively charged amino acid; replacement of Y349 with a negatively charged amino acid; replacement of L351 with a hydrophobic amino acid, and replacement of E357 with a hydrophobic amino acid; replacement of S364 with a hydrophobic amino acid; replacement of W366 with a hydrophobic amino acid, and replacement of K409 with a negatively charged amino acid; replacement of L368 with a hydrophobic amino acid; replacement of K370 with a negatively charged amino acid; replacement of K370 with a negatively charged amino acid, and replacement of K439 with a negatively charged amino acid; replacement of K392 with a negatively charged amino acid; replacement of T394 with a hydrophobic amino acid; replacement of V397 with a hydrophobic amino acid; replacement of D399 with a positively charged amino acid, and replacement of K409 with a negatively charged amino acid; replacement of S400 with a positively charged amino acid; F405W; Y407W; and replacement of K439 with a negatively charged amino acid.

In one embodiment the hydrophobic amino acid is selected from Norleucine, Met, Ala, Val, Leu, Ile, Trp, Tyr, and Phe. In one embodiment the hydrophobic amino acid is selected from Ala, Val, Leu, Ile and Tyr. In one embodiment the hydrophobic amino acid is Val, Leu, or Ile. In one embodiment the hydrophobic amino acid is Leu or Ile. In one embodiment the hydrophobic amino acid is Leu. In one embodiment the hydrophobic amino acid is Tyr. In one embodiment the hydrophobic amino acid is Phe.

In one embodiment the positively charged amino acid is His, Lys, or Arg. In one embodiment the positively charged amino acid is Lys, or Arg. In one embodiment the positively charged amino acid is Lys.

In one embodiment the negatively charged amino acid is Asp or Glu. In one embodiment the negatively charged amino acid is Asp. In one embodiment the negatively charged amino acid is Glu.

Amino acid substitutions with amino acids having the respective side-chain properties at the indicated amino acid positions in the CH3 domain were found to support polypeptide chain exchange and product polypeptide formation from two precursor polypeptides.

In one embodiment of the invention, the CH3 domain with the hole mutation comprises at least one amino acid substitution selected from the group of S354V, S354I, S354L, D356K, D356R, E357K, E357R, E357F, S364L, S364I, A368F, K392D, K392E, T394L, T394I, V407Y, K409E, K409D, K439D, K439E and a double mutation D399A S400K, D399A S400R, D399A F405W; and the CH3 domain with the knob mutation comprises at least one amino acid substitution selected from the group of Y349E, Y349D, S364V, S364I, S364L, L368F, K370E, K370D, K392E, K392D, T394L, T394I, V397Y, S400K, S400R, F405W, Y407W, K349E, K439D and double mutations Q347K K360E, Q347R K360E, Q347K K360D, Q347R K360D, L351F E357F, W366I K409E, W366L K409E, W366K K409D, W366L K409D, D399K K409E, D399R K409E, D399K K409D, and D399K K409E.

In one embodiment of the invention, the CH3 domain with the hole mutation comprises at least one amino acid substitution selected from the group of S354V, D356K, E357K, E357F, S364L, A368F, K392E, T394I, V407Y, K409E, K439E and a double mutation D399A S400K; and the CH3 domain with the knob mutation comprises at least one amino acid substitution selected from the group of Y349E, S364V, L368F, K370E, K392D, T394I, V397Y, S400K, F405W, Y407W, K349E, and double mutations Q347K K360E, L351F E357F, W366I K409E, and D399K K409E.

In one embodiment of the invention, the CH3 domain with the hole mutation comprises at least one amino acid substitution selected from the group of D356K, D356R, E357K, E357R, E357F, S364L, S364I, V407Y, K409E, K409D and a double mutation D399A S400K, D399A S400R; and the CH3 domain with the knob mutation comprises at least one amino acid substitution selected from the group of Y349E, Y349D, K370E, K370D, K392E, K392D, T394L, T394I, V397Y, F405W, Y407W, K349E, K439D and double mutations Q347K K360E, Q347R K360E, Q347K K360D, Q347R K360D, W366I K409E, W366L K409E, W366K K409D, W366L K409D, D399K K409E, D399R K409E, D399K K409D, and D399K K409E.

In one embodiment of the invention, the CH3 domain with the hole mutation comprises at least one amino acid substitution selected from the group of D356K, E357K, E357F, S364L, V407Y, K409E, and a double mutation D399A S400K; and the CH3 domain with the knob mutation comprises at least one amino acid substitution selected from the group of Y349E, K370E, K392D, T394I, V397Y, F405W, Y407W, K349E, and double mutations Q347K K360E, W366I K409E, and D399K K409E.

In one embodiment of the invention, the CH3 domain with the hole mutation and the CH3 domain with the knob mutation that comprise the destabilizing mutations comprise one of the amino acid substitutions selected from the group indicated in the following table:

| CH3 domain comprising | CH3 domain comprising |
|---|---|
| hole mutation | knob mutation |
| D356K | K370E K439E |
| D356K E357K | Y349E |
| D356K E357K | W366I K409E |
| D356K E357K | K370E K439E |
| D356K E357K | K392D |
| D356K E357K | T394I |
| D356K E357K | D399K K409E |
| D356K E357K | F405W |
| D356K E357K | Y407W |
| E357K | Y349E |
| E357K | W366I K409E |
| E357K | K370E K439E |
| E357K | T394I |
| E357K | D399K K409E |
| E357K | F405W |
| E357K | Y407W |
| E357F | Q347K K360E |
| E357F | Y349E |
| E357F | W366I K409E |
| E357F | K370E K439E |
| E357F | T394I |
| E357F | D399K K409E |
| E357F | Y407W |
| S364L | Y349E |
| S364L | K392D |
| S364L | F405W |
| S364L | W366I K409E |
| S364L | K370E K439E |
| S364L | T394I |
| S364L | D399K K409E |
| S364L | Y407W |
| A368F | T394I |
| D399A S400K | W366I K409E |
| D399A S400K | T394I |
| V407Y | Q347K K360E |
| V407Y | Y349E |
| V407Y | W366I K409E |
| V407Y | T394I |
| V407Y | D399K K409E |
| V407Y | F405W |
| V407Y | Y407W |
| V407Y | K392D |
| K409E | Y349E |
| K409E | W366I K409E |
| K409E | K392D |
| K409E | T394I |
| K409E | D399K K409E |
| K409E | F405W |
| K409E | Y407W |

For clarity, this table is to be understood in that the CH3 domain comprising the hole mutation comprises a destabilizing mutation as indicated in the first column of above table, the CH3 domain comprising the knob mutation comprises the destabilizing mutation listed in the right column of above table, indicated in the same line.

In one embodiment of the invention, the CH3 domain with the hole mutation and the CH3 domain with the knob mutation that comprise the destabilizing mutations comprise one of the amino acid substitutions selected from the group indicated in the following table:

| CH3 domain comprising | CH3 domain comprising |
|---|---|
| hole mutation | knob mutation |
| V407Y | K370E |
| V407Y | K370E K439E |
| V407Y | V397Y |
| S364L | K439E |
| S364L | W366I K409E |
| S364L | K370E K439E |
| S364L | D399K K409E |
| S364L | T394I |
| S364L | W366I K409E |
| S364L | K370E K439E |
| S364L | D399K K409E |
| S364L | T394I |

In one embodiment of the invention, the CH3 domain with the hole mutation and the CH3 domain with the knob mutation that comprise the destabilizing mutations comprise one of the amino acid substitutions selected from the group indicated in the following table:

| CH3 domain comprising | CH3 domain comprising |
|---|---|
| hole mutation | knob mutation |
| T394I E357F | Y349E Y407W |
| S364L E357F | Y349E Y407W |
| E357K T394I | Y349E K439E |
| E357K T394I | Y349E W366I K409E |
| D356K V407Y | W366I K409E F405W |
| S364L E357F | W366I K409E K439E |
| T394I E357F | W366I K409E K439E |
| S364L D356K | W366I K409E K439E |
| D356K V407Y | T394I F405W |
| S364L D356K | T394I F405W |

### 3^{rd} Set of Mutations (PACE2.0 Mutations)

A third set of destabilizing mutations has been disclosed in PCT/EP2020/061413.

In one embodiment of the invention, the CH3 domain with the hole mutation comprises at least one amino acid substitution, i.e. destabilizing mutation, selected from the group of replacement of E357 with a positively charged amino acid; replacement of S364 with a hydrophobic amino acid; replacement of A368 with a hydrophobic amino acid; and replacement of V407 with a hydrophobic amino acid; and the CH3 domain with the knob mutation either does not comprise a destabilizing mutation, or comprises at least one amino acid substitution, i.e. destabilizing mutation, selected from the group of replacement of K370 with a negatively charged amino acid; replacement of K370 with a negatively charged amino acid, and replacement of K439 with a negatively charged amino acid; replacement of K392 with a negatively charged amino acid; and replacement of V397 with a hydrophobic amino acid.

In one embodiment the hydrophobic amino acid is selected from Norleucine, Met, Ala, Val, Leu, Ile, Trp, Tyr, and Phe. In one embodiment the hydrophobic amino acid is selected from Ala, Val, Leu, Ile and Tyr. In one embodiment the hydrophobic amino acid is Val, Leu, or Ile. In one embodiment the hydrophobic amino acid is Leu or Ile. In one embodiment the hydrophobic amino acid is Leu. In one embodiment the hydrophobic amino acid is Tyr. In one embodiment the hydrophobic amino acid is Phe.

In one embodiment the positively charged amino acid is His, Lys, or Arg. In one embodiment the positively charged amino acid is Lys, or Arg. In one embodiment the positively charged amino acid is Lys.

In one embodiment the negatively charged amino acid is Asp or Glu. In one embodiment the negatively charged amino acid is Asp. In one embodiment the negatively charged amino acid is Glu.

Amino acid substitutions with amino acids having the respective side-chain properties at the indicated amino acid positions in the CH3 domain were found to support polypeptide chain exchange and product polypeptide formation from two precursor polypeptides.

In one embodiment of the invention, the CH3 domain with the hole mutation comprises at least one amino acid substitution selected from the group of E357K, E357R, S364L, S364I, V407Y, V407F and A368F; and the CH3 domain with the knob mutation either does not comprise a destabilizing mutation, or comprises at least one amino acid substitution selected from the group of K370E, K370D, K392E, K392D, V397Y, and double mutations K370E K439E, K370D K439E, K370E K439D, and K370D K439D.

In one embodiment of the invention, the CH3 domain with the hole mutation comprises at least one amino acid substitution selected from the group of E357K, S364L, V407Y and A368F; and the CH3 domain with the knob mutation comprises at least one amino acid substitution selected from the group of K370E, K392D, V397Y, and double mutation K370E K439E.

In one embodiment of the invention, the CH3 domain with the hole mutation comprises at least one amino acid substitution selected from the group of E357K, E357R, S364L, S364I, V407Y, and V407F; and the CH3 domain with the knob mutation comprises at least one amino acid substitution selected from the group of K370E, K370D, K392E, K392D, V397Y, and double mutations K370E K439E, K370D K439E, K370E K439D, and K370D K439D.

In one embodiment of the invention, the CH3 domain with the hole mutation comprises at least one amino acid substitution selected from the group of E357K, S364L, and V407Y; and the CH3 domain with the knob mutation comprises at least one amino acid substitution selected from the group of K370E, K392D, V397Y, and double mutation K370E K439E.

In one embodiment of the invention, the CH3 domain with the hole mutation and the CH3 domain with the knob mutation that comprise the destabilizing mutations comprise one of the amino acid substitutions selected from the group indicated in the following table:

| CH3 domain comprising | CH3 domain comprising |
|---|---|
| hole mutation | knob mutation |
| E357K | V397Y |
| E357K | K370E |
| E357K | K392D |
| E357K | K370E K439E |
| V407Y | no destabilizing mutation |
| V407Y | V397Y |
| V407Y | K370E |
| S364L | no destabilizing mutation |
| S364L | V397Y |
| S364L | K370E |
| A368F | V397Y |
| A368F | K370E |
| A368F | K392D |
| A368F | K370E K439E |

For clarity, this table is to be understood in that the CH3 domain comprising the hole mutation comprises a destabilizing mutation as indicated in the first column of above table, the CH3 domain comprising the knob mutation comprises the destabilizing mutation listed in the right column of above table, indicated in the same line.

In one embodiment of the invention, the CH3 domain with the hole mutation and the CH3 domain with the knob mutation that comprise the destabilizing mutations comprise one of the amino acid substitutions selected from the group indicated in the following table:

| CH3 domain comprising | CH3 domain comprising |
|---|---|
| hole mutation | knob mutation |
| E357K | V397Y |
| E357K | K370E |
| E357K | K392D |
| E357K | K370E K439E |
| V407Y | V397Y |
| V407Y | K370E |
| S364L | V397Y |
| S364L | K370E |

b. Antigen Binding Sites

As outlined above, the first and second precursor protein of the invention are capable of undergoing polypeptide chain exchange. In certain embodiments, polypeptide chain exchange spontaneously occurs by providing both precursor proteins in solution. In certain embodiments, the first and second precursor proteins of the invention polypeptide chain exchange is supported when both precursor proteins are brought into close proximity, e.g. by having bound to the surface of the same cell. Hence, in one embodiment the precursor proteins are capable of undergoing polypeptide chain exchange when bound to the surface of a cell. In one embodiment the first precursor protein and the second precursor protein specifically bind to a target cell. In one embodiment the first precursor protein and the second precursor protein specifically bind to an antigen on a surface of a target cell. In one embodiment the first precursor protein and the second precursor protein specifically bind to different antigens on the surface of a target cell. A “target cell” as used herein, is a cell desired to undergo therapy with the proteins of the invention. In one embodiment the target cell is a cancer cell.

In one embodiment the first precursor protein and the second precursor protein comprise an antibody binding region. The antibody binding region may be arranged N-terminally or C-terminally to the dimerization domain.

In one embodiment of the invention the antigen binding region comprises a pair of a VH domain and a VL domain, which form an antigen binding site specifically binding to a target antigen.

In one embodiment the first precursor protein and the second precursor protein comprise an antibody fragment. In one embodiment, each precursor protein comprises an antibody fragment, which may be a single chain antibody fragment or an antibody fragment comprising two polypeptides.

In one embodiment of the invention the antibody fragment comprised in a (precursor) polypeptide according to the invention is an antibody fragment selected from the group of Fv, Fab, Fab, Fab-SH, F(ab)₂, diabodies, scFv, and scFab. In one embodiment the antibody fragment comprised in a (precursor) polypeptide according to the invention is a Fv or a Fab. In one embodiment of the invention, the antigen binding region is a Fab fragment. In one embodiment of the invention, the first antigen binding region is a first Fab fragment and the second antigen binding region is a second Fab fragment.

In one embodiment, in case the antibody fragment is a Fab fragment, the precursor protein comprises three polypeptides: an antibody light chain comprising VL-CL domains, an antibody-heavy-chain-like polypeptide comprising the corresponding VH-CH1 domains to allow formation of the functional Fab fragment and a CH3 domain, another antibody-heavy-chain-like polypeptide comprising the corresponding CH3 domain. As indicated above, further antibody domains, like CH2 domains or VH/VL pair may be present.

In one embodiment of the invention, the first Fab fragment, the second Fab fragment or both, the first and the second Fab fragment are altered by a domain crossover, such that either:
a) only the CH1 and CL domains are replaced by each other;
b) only the VH and VL domains are replaced by each other; or
c) the CH1 and CL domains are replaced by each other and the VH and VL domains are replaced by each other.

In one embodiment of the invention, the antigen binding region is a Fv fragment. In one embodiment of the invention, the first antigen binding region is a first Fv fragment and the second antigen binding region is a second Fv fragment.

In one embodiment of the invention, the antigen binding region of the first precursor protein and the antigen binding region of the second precursor protein bind to the same antigen. In one embodiment of the invention, the antigen binding region of the first precursor protein and the antigen binding region of the second precursor protein are identical antigen binding moieties.

In one embodiment of the invention, the antigen binding region of the first precursor protein and the antigen binding region of the second precursor protein bind to different antigens. In this case, upon polypeptide chain exchange between two precursor proteins, a multispecific product polypeptide is formed, which comprises the antigen binding region originating from the first precursor protein and the antigen binding region originating from the second precursor protein.

Further antigen binding moieties may be present in the precursor protein, which may be fused to the N-terminus or the C-terminus of a polypeptide chain comprised in the precursor protein in order to provide product polypeptide of higher valence.

Such further antigen binding moieties are fused to the polypeptide chain via an appropriate peptide connector. In one embodiment the peptide connector is a glycine serine linker.

In one embodiment of the invention in a precursor protein only one of the polypeptide chains comprising a CH3 domain of comprises at least a part of an antigen binding region. In one embodiment of the invention in a precursor protein one of the polypeptide chains comprising a CH3 domain of an antigen binding site specifically binding to a target antigen. In one embodiment of the invention in a precursor protein one of the polypeptide chains comprising the CH3 domain comprises from N- to C-terminal direction a hinge region, an antibody variable domain and a CH3 domain, and the polypeptide chain is not part of an antigen binding site specifically binding to a target antigen. In one embodiment of the invention in a precursor protein one of the polypeptide chains comprising the CH3 domain comprises from N- to C-terminal direction a hinge region, an antibody variable domain, a CH2 domain and a CH3 domain, and the polypeptide chain is not part of an antigen binding site specifically binding to a target antigen.

c. Domain Arrangement of Precursor Proteins

Precursor polypeptides according to the invention are suitable for the generation of product proteins of various formats and with various domain arrangements. Depending on the selection of domains and the number of antigen binding regions provided in the precursor proteins, product polypeptides with different antigen binding characteristics (e.g. specificity, valency) and different effector functions may be generated.

In one embodiment the first precursor polypeptide and the second precursor protein comprise exactly two polypeptide chains comprising a CH3 domain. Thus, further polypeptide chains devoid of CH3 domains may be comprised in the first and second precursor protein.

### Antibody Fragment

In one embodiment of the invention the antigen binding region comprises a pair of a VH domain and a VL domain, which form an antigen binding site specifically binding to a target antigen; and
a) the first precursor protein comprises:
   a first heavy chain polypeptide comprising a CH3 domain and a first antibody variable domain,
   a second heavy chain polypeptide comprising a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
   a light chain polypeptide comprising a second antibody variable domain, wherein the first and second antibody variable domain together form a first antigen binding site specifically binding to a target antigen; and wherein
b) the second precursor protein comprises:
   a third heavy chain polypeptide comprising a CH3 domain and a third antibody variable domain,
   a fourth heavy chain polypeptide comprising a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
   a light chain polypeptide comprising a fourth antibody variable domain, wherein the third and fourth antibody variable domain together form a second antigen binding site specifically binding to a target antigen; and wherein
c) either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation.

### Precursor Proteins Comprising CH2 Domain

In one embodiment of the invention, the first precursor protein and the second precursor protein comprise at least two polypeptide chains comprising a CH2 domain and the CH3 domain. Precursor proteins comprising CH2 domains and CH3 domains exhibit advantageous properties, such as long half-life in the circulation and mediation of Fc mediated effector function.

In one embodiment of the invention, the first precursor protein and the second precursor protein comprise at least two polypeptide chains comprising from N- to C-terminal direction a CH2 domain and the CH3 domain.

In one embodiment of the invention, either i) the first precursor protein comprises one polypeptide chain comprising a VL domain, a CH2 domain and the CH3 domain, and wherein the second precursor protein comprises one polypeptide chain comprising a VH domain, a CH2 domain and the CH3 domain, wherein said VL domain and said VH domain specifically bind to an antigen when associated to a pair of a VH domain and a VL domain; or ii) the first precursor protein comprises one polypeptide chain comprising a VH domain, a CH2 domain and the CH3 domain, and wherein the second precursor protein comprises one polypeptide chain comprising a VL domain, a CH2 domain and the CH3 domain, wherein said VL domain and said VH domain specifically bind to an antigen when associated to a pair of a VH domain and a VL domain.

In one embodiment of the invention, the first precursor protein and the second precursor protein are devoid of a CH2 domain. Precursor proteins devoid of CH2 domains may exhibit advantageous properties, such as fast clearance from the circulation.

### Activatable Antigen Binding Site

In certain embodiments, each precursor protein comprises a part of an antigen binding region, wherein said antigen binding region is non-functional in the precursor polypeptide, and wherein in the product polypeptide formed by polypeptide chain exchange between the precursor polypeptides the antigen binding region is functional and specifically binds to a target antigen. In certain embodiments, precursor proteins of the inventions comprise an additional pair of VH and VL domains that is functionally active only after polypeptide chain exchange between the precursor proteins, i.e. in the activated protein. The activation of an antigen binding site by polypeptide chain exchange has been described before in WO2019086362, PCT/EP2020/061412 and PCT/EP2020/061413. In brief, one precursor protein comprises a VH domain derived from an antibody of interest, that is paired either with a CH2 domain or with a VL domain from a different antibody. In both cases, no functional binding site is formed. Yet, the other precursor protein comprises the corresponding VL domain derived from the antibody of interest, that is paired either with a CH2 domain or with a VH domain from a different antibody. Upon polypeptide chain exchange, both variable domains VH and VL of the antibody of interest are combined within the activated antibody. In one embodiment the antibody of interest specifically binds to a T cell antigen, in one embodiment CD3. For this, both variable domains have to be arranged on polypeptides having CH3 domains, wherein the VH domain is arranged on the CH3 domain having a knob mutation and the VL domain is arranged on the CH3 domain having a hole mutation; or vice versa (i.e. VH domain on the CH3-hole polypeptide and VL on the CH3-knob polypeptide). Hence, in one embodiment a) the activated protein comprises a pair of a VH domains and a VL domain specifically binding to an antigen, wherein the VH domain is comprised in the polypeptide from the first precursor protein and the VL domain is comprised in the polypeptide from the second precursor protein; or b) the activated protein comprises a pair of a VH domains and a VL domain specifically binding to an antigen, wherein the VL domain is comprised in the polypeptide from the first precursor protein and the VH domain is comprised in the polypeptide from the second precursor protein. In one embodiment the antigen is a T cell antigen, preferably CD3.

In one embodiment of the invention said antigen binding region is an antigen binding site comprising a pair of antibody variable domains.

In one embodiment of the invention the first precursor protein comprises one polypeptide chain comprising a VL domain and the CH3 domain, and wherein the second precursor protein comprises one polypeptide chain comprising a VH domain and the CH3 domain, wherein said VL domain and said VH domain specifically bind to an antigen when associated to a pair of a VH domain and a VL domain. In one embodiment the antigen specifically bound by the pair of the VH domain and the VL domain is CD3.

In one embodiment of the invention the first precursor protein comprises one polypeptide chain comprising from N- to C-terminal direction a VL domain and the CH3 domain, and wherein the second precursor protein comprises one polypeptide chain comprising from N- to C-terminal direction a VH domain and the CH3 domain, wherein said VL domain and said VH domain specifically bind to an antigen when associated to a pair of a VH domain and a VL domain.

In one embodiment of the invention
a) the first precursor protein comprises:
   a) a first heavy chain polypeptide comprising from N- to C-terminal direction a first VH domain, a CHI domain, a second antibody variable domain selected from a VH domain and a VL domain, and a CH3 domain,
   b) a second heavy chain polypeptide comprising from N- to C-terminal direction an antibody variable domain capable of associating with the second antibody variable domain of the first heavy chain polypeptide, and a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
   c) a light chain polypeptide comprising from N- to C-terminal direction a first VL domain and a CL domain, wherein the first VH domain and the first VL domain are associated with each other and form an antigen binding site specifically binding to a target antigen; and wherein
b) the second precursor protein comprises:
   d) a third heavy chain polypeptide comprising from N- to C-terminal direction a second VH domain, a CHI domain, a third antibody variable domain selected from a VH domain and a VL domain, and a CH3 domain,
   e) a fourth heavy chain polypeptide comprising from N- to C-terminal direction an antibody variable domain capable of associating with the third antibody variable domain of the third heavy chain polypeptide, and a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
   f) a light chain polypeptide comprising from N- to C-terminal direction a second VL domain and a CL domain, wherein the second VH domain and the second VL domain are associated with each other and form an antigen binding site specifically binding to a target antigen; and wherein
c) either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation; and wherein
d) the variable domains of the first heavy chain polypeptide and the third heavy chain polypeptide are capable of forming an antigen binding site specifically binding to a target antigen.

In one embodiment the first heavy chain polypeptide comprises from N- to C-terminal direction a first VH domain, a CH1 domain, a second antibody variable domain selected from a VH domain and a VL domain, a peptide connector and a CH3 domain, and the second heavy chain polypeptide comprising from N- to C-terminal direction an antibody variable domain capable of associating with the second antibody variable domain of the first heavy chain polypeptide, a peptide connector and a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and the third heavy chain polypeptide comprises from N- to C-terminal direction a second VH domain, a CH1 domain, a third antibody variable domain selected from a VH domain and a VL domain, a peptide connector and a CH3 domain, and the fourth heavy chain polypeptide comprises from N- to C-terminal direction an antibody variable domain capable of associating with the third antibody variable domain of the third heavy chain polypeptide, a peptide connector and a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation. In one embodiment the peptide connectors comprised in the first, second, third and fourth heavy chain polypeptides are identical.

In one embodiment, within the first precursor protein the second antibody variable domain comprised the first heavy chain polypeptide is derived from an antibody specifically binding to a first target antigen, and the antibody variable domain comprised in the second heavy chain polypeptide specifically binds to a second target antigen. Both variable domains are capable of associating with each other. Thus, one of the heavy chain polypeptides comprises a VH domain while the other heavy chain polypeptides comprises a VL domain. The VH domain and the VL domain are capable of associating with each other. However, a non-functional antigen binding site is formed. Thus the term “variable domains capable of associating with each other” within the context of the invention means that a pair of a VH and a VL domain is provided. In this embodiment, within the second precursor protein the third antibody variable domain comprised the third heavy chain polypeptide is derived from an antibody specifically binding to a first target antigen (i.e. is capable of forming a functional VH/VL pair with the second variable domain comprised in the first heavy chain polypeptide of the first precursor protein), and the antibody variable domain comprised in the fourth heavy chain polypeptide specifically binds to another, e.g. second, target antigen. The variable domains comprised in the first heavy chain polypeptide and the third heavy chain polypeptide are capable of associating with each other, i.e. one of the variable domains is a VH domain and the other one of the variable domains is a VL domain; and the variable domains comprised in the first heavy chain polypeptide and the third heavy chain polypeptide are capable of forming an antigen binding site specifically binding to a target antigen, i.e. both variable domains are derived from the same antibody specifically binding to the target antigen, e.g. CD3.

In one embodiment of the invention the first precursor protein and the second precursor protein comprise at least two polypeptide chains comprising from N- to C-terminal direction a CH2 domain and the CH3 domain, wherein the first precursor protein comprises one polypeptide chain comprising from N- to C-terminal direction a VL domain, a CH2 domain and the CH3 domain, and wherein the second precursor protein comprises one polypeptide chain comprising from N- to C-terminal direction a VH domain, a CH2 domain and the CH3 domain, wherein the VL domain and the VH domain are capable of forming an antigen binding site specifically binding to a target antigen.

In one embodiment of the invention
a) the first precursor protein comprises:
   a) a first heavy chain polypeptide comprising from N- to C-terminal direction a first VH domain, a CHI domain, a second antibody variable domain selected from a VH domain and a VL domain, a CH2 domain and a CH3 domain,
   b) a second heavy chain polypeptide comprising from N- to C-terminal direction an antibody variable domain capable of associating with the second antibody variable domain of the first heavy chain polypeptide, a CH2 domain and a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and
   c) a light chain polypeptide comprising from N- to C-terminal direction a first VL domain and a CL domain, wherein the first VH domain and the first VL domain are associated with each other and form an antigen binding site specifically binding to a target antigen; and wherein
b) the second precursor protein comprises:
   d) a third heavy chain polypeptide comprising from N- to C-terminal direction a second VH domain, a CH1 domain, a third antibody variable domain selected from a VH domain and a VL domain, a CH2 domain and a CH3 domain,
   e) a fourth heavy chain polypeptide comprising from N- to C-terminal direction an antibody variable domain capable of associating with the third antibody variable domain of the third heavy chain polypeptide, a CH2 domain and a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation; and
   f) a light chain polypeptide comprising from N- to C-terminal direction a second VL domain and a CL domain, wherein the second VH domain and the second VL domain are associated with each other and form an antigen binding site specifically binding to a target antigen; and wherein
c) either i) the first heavy chain polypeptide comprises a CH3 domain comprising a knob mutation and the third heavy chain polypeptide comprises a CH3 domain comprising a hole mutation; or ii) the first heavy chain polypeptide comprises a CH3 domain comprising a hole mutation and the third heavy chain polypeptide comprises a CH3 domain comprising a knob mutation; and wherein
d) the variable domains of the first heavy chain polypeptide and the third heavy chain polypeptide are capable of forming an antigen binding site specifically binding to a target antigen.

In one embodiment the first heavy chain polypeptide comprises from N- to C-terminal direction a first VH domain, a CH1 domain, a second antibody variable domain selected from a VH domain and a VL domain, a peptide connector, a CH2 domain and a CH3 domain, and the second heavy chain polypeptide comprising from N- to C-terminal direction an antibody variable domain capable of associating with the second antibody variable domain of the first heavy chain polypeptide, a peptide connector, a CH2 domain and a CH3 domain, wherein the first heavy chain polypeptide and the second heavy chain polypeptide are associated with each other via the CH3 domains and form a heterodimer, wherein one of the CH3 domains comprises a knob mutation and the other CH3 domain comprises a hole mutation; and the third heavy chain polypeptide comprises from N- to C-terminal direction a second VH domain, a CH1 domain, a third antibody variable domain selected from a VH domain and a VL domain, a peptide connector, a CH2 domain and a CH3 domain, and the fourth heavy chain polypeptide comprises from N- to C-terminal direction an antibody variable domain capable of associating with the third antibody variable domain of the third heavy chain polypeptide, a peptide connector, a CH2 domain and a CH3 domain, wherein the third heavy chain polypeptide and the fourth heavy chain polypeptide are associated with each other via the CH3 domains and form a heterodimer, wherein one of the CH3 domains comprises a knob mutation and the other CH3 domain comprises a hole mutation. In one embodiment the peptide connectors comprised in the first, second, third and fourth heavy chain polypeptides are identical.

### Interchain disulfide Bonds

As the precursor proteins of the invention have an antibody-based structure, interchain disulfides between individual polypeptides of a precursor protein may be present. However, polypeptide chain exchange between polypeptides connected via interchain disulfide only occurs after reduction of the disulfide bonds, which is undesired for therapeutic use. Hence, for therapy, precursor proteins are devoid of interchain disulfide bonds between the polypeptides comprising the dimerization domains. In order to realize this, naturally occurring disulfide bonds are removed by suitable amino acid mutations (additions, deletions, substitutions) known in the art.

### Hinge Region

In one embodiment the first precursor protein and the second precursor protein comprise a hinge region. In one embodiment the first precursor protein and the second precursor protein do not comprise an interchain disulfide bond in the hinge region.

In one embodiment of the invention, the first precursor protein and the second precursor protein comprise at least two polypeptide chains comprising from N- to C-terminal direction a hinge region and the CH3 domain.

In one embodiment of the invention, the first precursor protein and the second precursor protein comprise at least two polypeptide chains comprising from N- to C-terminal direction a hinge region, a CH2 domain and the CH3 domain.

In one embodiment of the invention, the first precursor protein and the second precursor protein do not comprise an interchain disulfide bond in the hinge region. Precursor proteins having a hinge region without interchain disulfide bonds are capable of undergoing a polypeptide chain exchange in absence of a reducing agent. Hence, precursor proteins having a hinge region without interchain disulfide bonds are particularly suitable for applications in which the presence of reducing agents is not possible or not desired. Thus, those precursor proteins may be advantageously used in therapy.

In one embodiment of the invention, the first precursor protein and the second precursor protein comprise a natural hinge region, which does not form interchain disulfides. One example is the hinge region peptide derived from an antibody of IgG4 isotype.

Instead of a hinge region without interchain disulfide bonds the precursor proteins may comprise a peptide connector, connecting the (part of the) antigen binding region with the constant antibody domain (i.e. CH2 or CH3). In one embodiment of the invention, no interchain disulfide bond is formed between the first and the second peptide connector. In one embodiment of the invention, the first and second peptide connectors are identical to each other.

In one embodiment of the invention, the first precursor protein and the second precursor protein comprise at least two polypeptide chains comprising from N- to C-terminal direction a peptide connector and the CH3 domain.

In one embodiment of the invention, the first precursor protein and the second precursor protein comprise at least two polypeptide chains comprising from N- to C-terminal direction a peptide connector, a CH2 domain and the CH3 domain.

In one embodiment of the invention, the first precursor protein comprises a first polypeptide chain comprising a first peptide connector, an antibody variable domain, optionally a CH2 domain, and the CH3 domain, and a second polypeptide chain comprising a first peptide connector, an antibody variable domain capable of associating with the antibody variable domain from the first polypeptide chain, optionally a CH2 domain, and the CH3 domain; and the second precursor protein comprises a first polypeptide chain comprising a first peptide connector, an antibody variable domain, optionally a CH2 domain, and the CH3 domain, and a second polypeptide chain comprising a first peptide connector, an antibody variable domain capable of associating with the antibody variable domain from the first polypeptide chain, optionally a CH2 domain, and the CH3 domain.

In one embodiment of the invention, the peptide connector is a peptide of at least 15 amino acids. In another embodiment of the invention, the peptide connector is a peptide of 15-70 amino acids. In another embodiment of the invention, the peptide connector is a peptide of 20-50 amino acids. In another embodiment of the invention, the peptide connector is a peptide of 10-50 amino acids. Depending e.g. on the type of antigen to be bound by the activatable binding site, shorter (or even longer) peptide connectors may also be applicable in precursor proteins according to the invention.

In yet another embodiment of the invention, the first and second peptide connector are approximately of the length of the natural hinge region (which is for natural antibody molecules of IgG1 isotype about 15 amino acids, and for IgG3 isotype about 62 amino acids). Therefore, in one embodiment, wherein the first precursor protein and the second precursor protein are of IgG1 isotype, the peptide connectors are peptides of 10-20 amino acids, in one preferred embodiment of 12-17 amino acids. In another one embodiment, wherein the first precursor protein and the second precursor protein are of IgG3 isotype, the peptide connectors are peptides of 55-70 amino acids, in one preferred embodiment of 60-65 amino acids.

In one embodiment of the invention, the peptide connector is a glycine-serine linker. In one embodiment of the invention, the peptide connector is a peptide consisting of glycine and serine residues. In one embodiment of the invention, the glycine-serine linkers are of the structure
(GxS)n or (GxS)nGm
with G=glycine, S=serine, x=3 or 4, n=2, 3, 4, 5 or 6, and m=0, 1, 2 or 3.

In one embodiment, of above defined glycine-serine linkers, x=3, n=3, 4, 5 or 6, and m=0, 1, 2 or 3; or x=4, n=2, 3, 4 or 5 and m=0, 1, 2 or 3. In one preferred embodiment, x=4 and n=2 or 3, and m=0. In yet another preferred embodiment, x=4 and n=2. In one embodiment said peptide connector is (G₄S)₄ or (G₄S)₆.

In one embodiment of the invention, either i) the first precursor protein comprises one polypeptide chain comprising a VL domain, a peptide connector and the CH3 domain, and wherein the second precursor protein comprises one polypeptide chain comprising a VH domain, a peptide connector and the CH3 domain, wherein said VL domain and said VH domain specifically bind to an antigen when associated to a pair of a VH domain and a VL domain; or ii) the first precursor protein comprises one polypeptide chain comprising a VH domain, a peptide connector and the CH3 domain, and wherein the second precursor protein comprises one polypeptide chain comprising a VL domain, a peptide connector and the CH3 domain, wherein said VL domain and said VH domain specifically bind to an antigen when associated to a pair of a VH domain and a VL domain.

In one embodiment of the invention, either i) the first precursor protein comprises one polypeptide chain comprising a VL domain, a peptide connector, a CH2 domain and the CH3 domain, and wherein the second precursor protein comprises one polypeptide chain comprising a VH domain, a peptide connector, a CH2 domain and the CH3 domain, wherein said VL domain and said VH domain specifically bind to an antigen when associated to a pair of a VH domain and a VL domain; or ii) the first precursor protein comprises one polypeptide chain comprising a VH domain, a peptide connector, a CH2 domain and the CH3 domain, and wherein the second precursor protein comprises one polypeptide chain comprising a VL domain, a peptide connector, a CH2 domain and the CH3 domain, wherein said VL domain and said VH domain specifically bind to an antigen when associated to a pair of a VH domain and a VL domain.

### Antibody Isotypes

In one embodiment of the invention, the precursor polypeptides comprise immunoglobulin constant regions of one or more immunoglobulin classes. Immunoglobulin classes include IgG, IgM, IgA, IgD, and IgE isotypes and, in the case of IgG and IgA, their subtypes. In one embodiment of the invention, the precursor polypeptide has a constant domain structure of an IgG type antibody.

In one embodiment of the invention the CH3 domains comprised in a precursor polypeptide are of mammalian IgG class. In one embodiment of the invention the CH3 domains comprised in a precursor polypeptide are of mammalian IgG1 subclass. In one embodiment of the invention the CH3 domains comprised in a precursor polypeptide are of mammalian IgG4 subclass.

In one embodiment of the invention the CH3 domains comprised in a precursor polypeptide are of human IgG class. In one embodiment of the invention the CH3 domains comprised in a precursor polypeptide are of human IgG1 subclass. In one embodiment of the invention the CH3 domains comprised in a precursor polypeptide are of human IgG4 subclass.

In one embodiment the constant domains of a precursor polypeptide according to the invention are of human IgG class. In one embodiment the constant domains of a precursor polypeptide according to the invention are of human IgG1 subclass. In one embodiment the constant domains of a precursor polypeptide according to the invention are of human IgG4 subclass.

In one embodiment, the precursor polypeptides are devoid of a CH4 domain.

In one embodiment of the invention the constant domains of a precursor polypeptide according to the invention are of the same immunoglobulin subclass. In one embodiment of the invention the variable domains and constant domains of a precursor polypeptide according to the invention are of the same immunoglobulin subclass.

In one embodiment of the invention the precursor polypeptide is an isolated precursor polypeptide. In one embodiment of the invention the product polypeptide is an isolated product polypeptide.

In one embodiment, a precursor protein or a heterodimeric product polypeptide comprising a polypeptide chain including a CH3 domain includes a full length CH3 domain or a CH3 domain, wherein one or two C-terminal amino acid residues, i.e. G446 and/or K447 are not present.

In one embodiment the first precursor protein is monospecific and comprises a part of a second antigen binding site; the second precursor protein is monospecific and comprises the other part of the second antigen binding site. In said embodiment the heterodimeric product polypeptide is bispecific or trispecific.

In one embodiment the first precursor protein is monospecific and comprises a part of a second antigen binding site; the second precursor protein is monospecific and comprises the other part of the second antigen binding site. In said embodiment the heterodimeric product polypeptide is trispecific.

In one embodiment the first precursor protein is bispecific. In one embodiment the second precursor protein is monospecific.

In one embodiment the first precursor protein is bispecific. In one embodiment the second precursor protein is bispecific.

In one embodiment the first precursor protein is monovalent. In one embodiment the second precursor protein is monovalent.

In one embodiment the first precursor protein is bivalent. In one embodiment the second precursor protein is bivalent.

In one embodiment the first precursor protein is trivalent. In one embodiment the second precursor protein is trivalent.

In one embodiment the heterodimeric product polypeptide is trivalent. In one embodiment the heterodimeric product polypeptide is tetravalent.

### E) Application in Therapy

In a second aspect the invention is directed to a therapeutic kit comprising a first precursor protein and a second precursor protein as defined above for the first aspect of the invention. In one embodiment the therapeutic kit comprises a first pharmaceutical composition comprising the first precursor protein and a second pharmaceutical composition comprising the second precursor protein. In one embodiment the therapeutic kit of the invention is for use as a medicament. One embodiment the therapeutic kit of the invention comprises a first precursor protein and a second precursor protein with an activatable antigen binding site specifically binding to CD3, wherein the first precursor protein and the second precursor protein comprise antigen binding regions specifically binding to an antigen on a cancer cell and is for use as a medicament in the treatment of cancer. In one embodiment the first precursor protein and the second precursor protein comprise antigen binding regions binding to different antigens on a cancer cell.

In a third aspect the invention is directed to the use of a set of a first precursor protein and a second precursor protein as defined above for the first aspect of the invention for the generation of an activated form of the moiety.

In a fourth aspect the invention is directed to the use of a set of a first precursor protein and a second precursor protein as defined above for the first aspect of the invention for therapy. In one embodiment the therapy is the treatment of cancer.

In a fifth aspect the invention is directed to a method for providing a therapeutic kit according to the second aspect of the invention, comprising the steps of providing recombinantly expressed first precursor protein and recombinantly expressed second precursor protein, and formulating the first and second precursor protein, optionally with a pharmaceutically acceptable carrier to provide the therapeutic kit.

Proteins according to the invention are produced by recombinant means. Methods for recombinant production of proteins, e.g. antibodies, are widely known in the state of the art and comprise protein expression in prokaryotic and eukaryotic host cells with subsequent isolation of the polypeptide and usually purification to a pharmaceutically acceptable purity. For the expression of the polypeptides as aforementioned in a host cell, nucleic acids encoding the respective polypeptide chains are inserted into expression vectors by standard methods. Expression is performed in appropriate prokaryotic or eukaryotic host cells, like CHO cells, NS0 cells, SP2/0 cells, HEK293 cells, COS cells, PER.C6 cells, yeast, or E. coli cells, and the polypeptide is recovered from the cells (supernatant or cells after lysis). General methods for recombinant production of polypeptides, e.g. antibodies, are well-known in the state of the art and described, for example, in the review articles of Makrides, S. C., Protein Expr. Purif. 17 (1999) 183-202; Geisse, S., et al., Protein Expr. Purif. 8 (1996) 271-282; Kaufman, R. J., Mol. Biotechnol. 16 (2000) 151-161; Werner, R. G., Drug Res. 48 (1998) 870-880.

### F) Method of Generating Activated Protein

In a sixth aspect the invention is directed to a method for generating an activated protein, comprising the steps of a) providing recombinantly expressed first precursor protein and recombinantly expressed second precursor protein, and b) combinding the first precursor protein and the second precursor protein under conditions that allow polypeptide chain exchange between the precursor proteins so that the activated protein is formed, wherein the activated protein comprises a polypeptide derived from the first precursor protein and a polypeptide derived from the second precursor protein.

In one embodiment of the sixth aspect the invention provides a method of generating a product protein, the method comprising contacting a first precursor protein and a second precursor protein according to the invention to form a third heterodimeric polypeptide comprising at least one polypeptide chain comprising a CH3 domain from the first precursor protein and at least one polypeptide chain comprising a CH3 domain from the second heterodimeric polypeptide. In one embodiment of the invention the method includes a step of recovering the third heterodimeric polypeptide.

In one embodiment the first precursor protein and the second precursor protein according to the invention are contacted to form a third heterodimeric polypeptide comprising at least one polypeptide chain comprising a CH3 domain from the first precursor protein and at least one polypeptide chain comprising a CH3 domain from the second heterodimeric polypeptide, and a fourth heterodimeric polypeptide comprising the other polypeptide comprising a CH3 domain from the first precursor protein and the other polypeptide comprising a CH3 domain from the second precursor protein. In one embodiment the method includes the step of recovering the fourth heterodimeric product polypeptide.

In one embodiment of the invention the method includes the formation of a third heterodimeric product polypeptide and a fourth heterodimeric product polypeptide, wherein one of the product polypeptides (i.e. either the third heterodimeric product polypeptide, or the fourth heterodimeric product polypeptide) does not comprise an antigen binding site specifically binding to an antigen.

In one embodiment of the invention the first precursor protein comprises an antigen binding moiety specifically binding to a first antigen and comprises a part of a second antigen binding site, wherein the second precursor protein comprises an antigen binding moiety specifically binding to the third antigen and comprises the other part of the second antigen binding site, and wherein the third heterodimeric polypeptide comprises an antigen binding moieties specifically binding to the first antigen, an antigen binding moiety specifically binding to the second antigen; and an antigen binding moiety specifically binding to the third antigen.

In one embodiment of the invention the first precursor protein and the second precursor protein comprise a hinge region that does not comprise an interchain disulfide bond. In this case, the polypeptide chain exchange may occur in absence of a reducing agent. Thus, in one embodiment the first precursor protein and the second precursor protein comprise a hinge region that does not comprise an interchain disulfide bond, and the first precursor protein and the second precursor protein are contacted in absence of a reducing agent.

In one embodiment of the invention no interchain disulfide bond is formed between the two polypeptide chains comprising the CH3 domains of the first and second heterodimeric polypeptide, and the contacting is performed in absence of a reducing agent.

In a seventh aspect the invention is directed to an activated protein, produced by a method according to the sixth aspect of the invention.

One aspect of the invention is an activated protein, in one embodiment a heterodimeric activated product protein, comprising at least two polypeptide chains comprising a CH3 domain, wherein the two polypeptide chains comprising the CH3 domains do not comprise a destabilizing mutation.

Another product of the method of generating a product polypeptide, and therefore another aspect of the invention, is a product polypeptide, preferably obtained by the method of the invention, comprising two polypeptide chains comprising a CH3 domain, wherein both of the CH3 domains do not comprise a destabilizing mutation.

### 3. Specific Embodiments of the Invention

In the following specific embodiments of the invention are listed.
1. A set of a first and a second precursor protein,
   wherein each precursor protein comprises two polypeptides that are associated with each other via dimerization domains,
   wherein at least one of the precursor proteins comprises a moiety selected from a receptor ligand and an enzyme, wherein said moiety is functionally inactive, wherein said moiety is fused to the dimerization domain,
   wherein upon polypeptide chain exchange between the first and the second precursor protein an activated protein is formed,
      wherein the activated protein comprises one polypeptide from the first precursor protein and one polypeptides from the second precursor protein, wherein both polypeptides are associated with each other via their dimerization domains, and wherein the activated protein comprises said moiety,
      characterized in that the activated protein comprises said moiety in functionally active form.
2. The set of a first and a second precursor protein according to embodiment 1, wherein either the first precursor protein or the second precursor protein comprise the moiety selected from a receptor ligand and an enzyme, wherein the moiety is bound to an inactivation moiety.
3. The set of a first and a second precursor protein according to embodiment 2, wherein the moiety is a receptor ligand and the inactivation moiety is the corresponding receptor or a ligand-binding subunit thereof.
4. The set of a first and a second precursor protein according to embodiment 3, wherein the receptor ligand is a cytokine and the inactivation moiety is the corresponding cytokine receptor or a cytokine-binding subunit thereof.
5. The set of a first and a second precursor protein according to embodiment 3, wherein the receptor ligand is IL-2v and the inactivation moiety is selected from a subunit of IL-2R, preferably IL-2Rbeta, IL-2Rgamma-chain, IL-2Rbeta gamma-chain.
6. The set of a first and a second precursor protein according to embodiment 1, wherein the first precursor protein and the second precursor protein comprise complementary subunits of the moiety selected from a receptor ligand and an enzyme.
7. The set of a first and a second precursor protein according to embodiment 6, wherein the first precursor protein comprises a first unmodified subunit of the moiety and a second subunit of the moiety, wherein the second subunit comprises an inactivating mutation; and wherein the second precursor protein comprises the second unmodified subunit of the moiety.
8. The set of a first and a second precursor protein according to embodiment 7, wherein the moiety is a receptor ligand.
9. The set of a first and a second precursor protein according to embodiment 8, wherein the receptor ligand is a cytokine, wherein the first precursor protein comprises a first subunit of the cytokine and a second subunit of the cytokine, wherein the second subunit comprises an inactivating mutation; and wherein the second precursor protein comprises the second unmodified subunit of the cytokine.
10. The set of a first and a second precursor protein according to embodiment 9, wherein the first precursor protein comprises a IL-12 p35 and IL-12 p40 comprising an inactivating mutation; and wherein the second precursor protein comprises the unmodified IL-12 p40.
11. The set of a first and a second precursor protein according to embodiment 10, wherein the first precursor protein comprises a IL-12 p35 and IL-12 p40 comprising an inactivating mutation; and wherein the second precursor protein comprises the unmodified IL-12 p40 and IL-12 p35 comprising an inactivating mutation.
12. The set of a first and a second precursor protein according to embodiment 1, wherein the first precursor protein and the second precursor protein comprise complementary parts of an artificially splitted moiety selected from a receptor ligand and an enzyme, wherein one of the complementary parts is inactivated.
13. The set of a first and a second precursor protein according to embodiment 12, wherein the moiety is a receptor ligand.
14. The set of a first and a second precursor protein according to embodiment 13, wherein the receptor ligand is a cytokine.
15. The set of a first and a second precursor protein according to embodiment 14, wherein the artificially splitted moiety is a split cytokine.
16. The set of a first and a second precursor protein according to embodiment 15, wherein the receptor ligand is an enzyme.
17. The set of a first and a second precursor protein according to embodiment 16, wherein the artificially splitted moiety is a split enzyme.
18. The set of a first and a second precursor protein according to any one of the preceding embodiments, wherein the dimerization domains are CH3 domains.
19. The set of a first and a second precursor protein according to embodiment 18, wherein the CH3 domains have a modified interface to support polypeptide chain exchance between the first and the second precursor protein.
20. The set of a first and a second precursor protein according to embodiment 18 or 19, wherein the first precursor protein and the second precursor protein each comprise two polypeptides comprising a CH3 domain, wherein one CH3 domains comprises a knob mutation and the other CH3 domain comprises a hole mutation.
21. The set of a first and a second precursor protein according to embodiment 20, wherein either i) the CH3 domain comprising the knob mutation of the first precursor protein comprises a cysteine mutation and the CH3 domain comprising the hole mutation of the second precursor protein comprises a cysteine mutation, or ii) wherein the CH3 domain comprising the hole mutation of the first precursor protein comprises a cysteine mutation and the CH3 domain comprising the knob mutation of the second precursor protein comprises a cysteine mutation.
22. The set of a first and a second precursor protein according to embodiment 20 or 22, wherein either i) the CH3 domain of the first precursor protein comprising the knob mutation and the CH3 domain of the second precursor protein comprising the hole mutation, or ii) the CH3 domain of the first precursor protein comprising the hole mutation and the CH3 domain of the second precursor protein comprising the knob mutation comprise at least one complementary destabilizing mutation, whereas the other two CH3 domains of the first and the second precursor polypeptide do not comprise a destabilizing mutation.
23. The set of a first and a second precursor protein according to any one of the preceding embodiments, wherein the precursor proteins are capable of undergoing polypeptide chain exchange when bound to the surface of a cell.
24. The set of a first and a second precursor protein according to any one of the preceding embodiments, wherein the first precursor protein and the second precursor protein specifically bind to an antigen on a surface of a target cell.
25. The set of a first and a second precursor protein according to any one of the preceding embodiments, wherein the first precursor protein and the second precursor protein comprise an antibody binding region.
26. The set of a first and a second precursor protein according to any one of the preceding embodiments, wherein the first precursor protein and the second precursor protein comprise an antibody fragment.
27. The set of a first and a second precursor protein according to any one of the preceding embodiments, wherein the first precursor protein and the second precursor protein comprise a hinge region.
28. The set of a first and a second precursor protein according to embodiment 27, wherein the first precursor protein and the second precursor protein do not comprise an interchain disulfide bond in the hinge region.
29. The set of a first and a second precursor protein according to any one of the preceding embodiments, wherein
   a) the activated protein comprises a pair of a VH domains and a VL domain specifically binding to an antigen, wherein the VH domain is comprised in the polypeptide from the first precursor protein and the VL domain is comprised in the polypeptide from the second precursor protein; or
   b) the activated protein comprises a pair of a VH domains and a VL domain specifically binding to an antigen, wherein the VL domain is comprised in the polypeptide from the first precursor protein and the VH domain is comprised in the polypeptide from the second precursor protein.
30. The set of a first and a second precursor protein according to embodiment 29, wherein the antigen is a T cell antigen, preferably CD3.
31. A therapeutic kit comprising a first and a second precursor protein as defined in any one of the preceding embodiments.
32. Use of a set of a first and a second precursor protein as defined in any one of embodiments 1 to 30 for the generation of an activated form of the moiety.
33. Use of a set of a first and a second precursor protein as defined in any one of embodiments 1 to 30 for therapy.
34. Method for providing a therapeutic kit of embodiment 31, comprising the steps of providing recombinantly expressed first precursor protein and recombinantly expressed second precursor protein, and formulating the first and second precursor protein, optionally with a pharmaceutically acceptable carrier to provide the therapeutic kit.
35. Activated protein, produced by polypeptide chain exchange between a first precursor protein and a second precursor protein as defined in any one of embodiments 1 to 30.
36. Method for generating an activated protein according to embodiment 35, comprising the step of combining a first and a second precursor protein as defined in any one of embodiments 1 to 30 under conditions that allow polypeptide chain exchange between the first and second precursor protein.
37. A pharmaceutical composition comprising the set of a first and a second precursor protein according to any one of embodiments 1 to 30 and a pharmaceutically acceptable carrier.
38. A method of treating an individual having a disease comprising administering to the individual an effective amount of the first and second precursor protein according to any one of embodiments 1 to 30 or the pharmaceutical composition according to embodiment 37.
39. The set of precursor proteins according to any one of embodiments 1 to 30, wherein in the first and second heterodimeric precursor polypeptide the VH domain and the VL domain indicated in B) are capable of forming an antigen binding site specifically binding to CD3 for use in the treatment of cancer.
40. A method of treating an individual having a cancer comprising administering to the individual an effective amount of the first and second precursor protein according to any one of embodiments 1 to 30, wherein in the first and second heterodimeric precursor polypeptide the VH domain and the VL domain indicated in B) are capable of forming an antigen binding site specifically binding to CD3.

DESCRIPTIQN QF THE AMINQ ACID SEQUENCES

| | |
|---|---|
| SEQ ID NO: 1 | [LeY-VH_CH1_Dig-VH_CH3-Knob] |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGGGSQVQLVESGGG |
| | LVKPGGSLRLSCAASGFTFSDYAMSWIRQAPGKGLEWVSSIN |
| | IGATYIYYADSVKGRFTISRDNAKNSLYLQMNSLRAEDTAVY |
| | YCARPGSPYEYDKAYYSMAYWGQGTTVTVSSGQPREPQVYTL |
| | PPSRDELTKNQVSLWCLVKGFYPSDIAVEWESNGQPENNYKT |
| | TPPVLDSDGSFFLYSKLTVDKSRWQQGNVFSCSVMHEALHNH |
| | YTQKSLSLSPGK |
| SEQ ID NO: 2 | [LeY-VL_Ckappa] |
| | DVLMTQSPLSLPVSLGDQASISCRSSQIIVHSNGNTYLEWYL |
| | QKPGQSPKLLIYKVSNRFSGVPDRFSGSGSGTDFTLKISRVE |
| | AEDLGVYYCFQGSHVPFTFGSGTKLEIKRTVAAPSVFIFPPS |
| | DEQLKSGTASVVCLLNNFYPREAKVQWKVDNALQSGNSQESV |
| | TEQDSKDSTYSLSSTLTLSKADYEKHKVYACEVTHQGLSSPV |
| | TKSFNRGEC |
| SEQ ID NO: 3 | [Dig-VL_CH3-Hole]-[Linker]-[Human interleukin-4] |
| | DIQMTQSPSSLSASVGDRVTITCRASQDIKNYLNWYQQKPGK |
| | APKLLIYYSSTLLSGVPSRFSGSGSGTDFTLTISSLQPEDFA |
| | TYYCQQSITLPPTFGGGTKVEIKGQPREPQVYTLPPSRDELT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSHKCDITLQEIIKTLNSLTEQKST |
| | LCTELTVTDIFAASKNTTEKETFCRAATVLRQFYSHHEKDTR |
| | CLGATAQQFHRHKQLIRFLKRLDRNLWGLAGLNSCPVKEANQ |
| | STLENFLERLKTIMREKYSKCSS |
| SEQ ID NO: 4 | [Dig-VL_CH3-Hole]-[Linker]-[Interleukin-4 DABC (circularly |
| | permutated)] |
| | DIQMTQSPSSLSASVGDRVTITCRASQDIKNYLNWYQQKPGK |
| | APKLLIYYSSTLLSGVPSRFSGSGSGTDFTLTISSLQPEDFA |
| | TYYCQQSITLPPTFGGGTKVEIKGQPREPQVYTLPPSRDELT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSVKEANQSTLENFLERLKTIMREK |
| | YSKCSSGGGSGGSHKCDITLQEIIKTLNSLTEQKSTLCTELT |
| | VTDIFAASKNTTEKETFCRAATVLRQFYSHHEKDTRCLGATA |
| | QQFHRHKQLIRFLKRLDRNLWGLAGLNSCP |
| SEQ ID NO: 5 | [CD38-VH_CH1_Dig-VH_CH3-Knob]-[Linker]-[Split |
| | interleukin-4 (BCD)] |
| | EVQLLESGGGLVQPGGSLRLSCAVSGFTFNSFAMSWVRQAPG |
| | KGLEWVSAISGSGGGTYYADSVKGRFTISRDNSKNTLYLQMN |
| | SLRAEDTAVYFCAKDKILWFGEPVFDYWGQGTLVTVSSASTK |
| | GPSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCGGGGSGGGGSGGGGSGGGGSQVQLVES |
| | GGGLVKPGGSLRLSCAASGFTFSDYAMSWIRQAPGKGLEWVS |
| | SINIGATYIYYADSVKGRFTISRDNAKNSLYLQMNSLRAEDT |
| | AVYYCARPGSPYEYDKAYYSMAYWGQGTTVTVSSGQPREPQV |
| | YTLPPSRDELTKNQVSLWCLVKGFYPSDIAVEWESNGQPENN |
| | YKTTPPVLDSDGSFFLYSKLTVDKSRWQQGNVFSCSVMHEAL |
| | HNHYTQKSLSLSPGKGGGGSGGGGSGGGGSTLCTELTVTDIF |
| | AASKNTTEKETFCRAATVLRQFYSHHEKDTRCLGATAQQFHR |
| | HKQLIRFLKRLDRNLWGLAGLNSCPVKEANQSTLENFLERLK |
| | TIMREKYSKSSS |
| SEQ ID NO: 6 | [CD38-VL_Ckappa] |
| | EIVLTQSPATLSLSPGERATLSCRASQSVSSYLAWYQQKPGQ |
| | APRLLIYDASNRATGIPARFSGSGSGTDFTLTISSLEPEDFA |
| | VYYCQQRSNWPPTFGQGTKVEIKASTKGPSVFPLAPSSKSTS |
| | GGTASVVCLLNNFYPREAKVQWKVDNALQSGNSQESVTEQDS |
| | KDSTYSLSSTLTLSKADYEKHKVYACEVTHQGLSSPVTKSFN |
| | RGEC |
| SEQ ID NO: 7 | [Biotin-VL_CH3-Hole-E357K]-[Linker]-[Split interleukin-4 |
| | (A) E9Q] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSHKSDITLQQIIKTLNSLTEQK |
| SEQ ID NO: 8 | [CD38-VH_CH1_Dig-VL_CH3-Hole]-[Linker]-[Split |
| | interleukin-4 (A)] |
| | EVQLLESGGGLVQPGGSLRLSCAVSGFTFNSFAMSWVRQAPG |
| | KGLEWVSAISGSGGGTYYADSVKGRFTISRDNSKNTLYLQMN |
| | SLRAEDTAVYFCAKDKILWFGEPVFDYWGQGTLVTVSSASTK |
| | GPSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCGGGGSGGGGSGGGGSGGGGSDIQMTQS |
| | PSSLSASVGDRVTITCRASQDIKNYLNWYQQKPGKAPKLLIY |
| | YSSTLLSGVPSRFSGSGSGTDFTLTISSLQPEDFATYYCQQS |
| | ITLPPTFGGGTKVEIKGQPREPQVYTLPPSRDELTKNQVSLS |
| | CAVKGFYPSDIAVEWESNGQPENNYKTTPPVLDSDGSFFLVS |
| | KLTVDKSRWQQGNVFSCSVMHEALHNHYTQKSLSLSPGKGGG |
| | GSGGGGSGGGGSHKSDITLQEIIKTLNSLTEQK |
| SEQ ID NO: 9 | [Biotin-VH_CH3-Knob-K370E]-[Linker]-[Split interleukin-4 |
| | (BCD) R88Q] |
| | QVQLVQSGAEVKKPGSSVKVSCKSSGFNNKDTFFQWVRQAPG |
| | QGLEWMGRIDPANGFTKYAQKFQGRVTITADTSTSTAYMELS |
| | SLRSEDTAVYYCARWDTYGAAWFAYWGQGTLVTVSSGQPREP |
| | QVYTLPPSRDELTKNQVSLWCLVEGFYPSDIAVEWESNGQPE |
| | NNYKTTPPVLDSDGSFFLYSKLTVDKSRWQQGNVFSCSVMHE |
| | ALHNHYTQKSLSLSPGKGGGGSGGGGSGGGGSTLCTELTVTD |
| | IFAASKNTTEKETFCRAATVLRQFYSHHEKDTRCLGATAQQF |
| | HRHKQLIRFLKRLDQNLWGLAGLNSCPVKEANQSTLENFLER |
| | LKTIMREKYSKSSS |
| SEQ ID NO: 10 | [Her2-VH_CH1_Dig-VH_CH3-Knob]-[Linker]-[Split |
| | interleukin-4 (BCD)] |
| | EVQLVESGGGLVQPGGSLRLSCAASGFNIKDTYIHWVRQAPG |
| | KGLEWVARIYPTNGYTRYADSVKGRFTISADTSKNTAYLQMN |
| | SLRAEDTAVYYCSRWGGDGFYAMDYWGQGTLVTVSSASTKGP |
| | SVFPLAPSSKSTSGGTAALGCLVKDYFPEPVTVSWNSGALTS |
| | GVHTFPAVLQSSGLYSLSSVVTVPSSSLGTQTYICNVNHKPS |
| | NTKVDKKVEPKSCGGGGSGGGGSGGGGSGGGGSQVQLVESGG |
| | GLVKPGGSLRLSCAASGFTFSDYAMSWIRQAPGKGLEWVSSI |
| | NIGATYIYYADSVKGRFTISRDNAKNSLYLQMNSLRAEDTAV |
| | YYCARPGSPYEYDKAYYSMAYWGQGTTVTVSSGQPREPQVYT |
| | LPPSRDELTKNQVSLWCLVKGFYPSDIAVEWESNGQPENNYK |
| | TTPPVLDSDGSFFLYSKLTVDKSRWQQGNVFSCSVMHEALHN |
| | HYTQKSLSLSPGKGGGGSGGGGSGGGGSTLCTELTVTDIFAA |
| | SKNTTEKETFCRAATVLRQFYSHHEKDTRCLGATAQQFHRHK |
| | QLIRFLKRLDRNLWGLAGLNSCPVKEANQSTLENFLERLKTI |
| | MREKYSKSSS |
| SEQ ID NO: 11 | [Her2-VL_Ckappa] |
| | DIQMTQSPSSLSASVGDRVTITCRASQDVNTAVAWYQQKPGK |
| | APKLLIYSASFLYSGVPSRFSGSRSGTDFTLTISSLQPEDFA |
| | TYYCQQHYTTPPTFGQGTKVEIKRTVAAPSVFIFPPSDEQLK |
| | SGTASVVCLLNNFYPREAKVQWKVDNALQSGNSQESVTEQDS |
| | KDSTYSLSSTLTLSKADYEKHKVYACEVTHQGLSSPVTKSFN |
| | RGEC |
| SEQ ID NO: 12 | [Her2-VH_CH1_Dig-VL_CH3-Hole]-[Linker]-[Split |
| | interleukin-4 (A)] |
| | EVQLVESGGGLVQPGGSLRLSCAASGFNIKDTYIHWVRQAPG |
| | KGLEWVARIYPTNGYTRYADSVKGRFTISADTSKNTAYLQMN |
| | SLRAEDTAVYYCSRWGGDGFYAMDYWGQGTLVTVSSASTKGP |
| | SVFPLAPSSKSTSGGTAALGCLVKDYFPEPVTVSWNSGALTS |
| | GVHTFPAVLQSSGLYSLSSVVTVPSSSLGTQTYICNVNHKPS |
| | NTKVDKKVEPKSCGGGGSGGGGSGGGGSGGGGSDIQMTQSPS |
| | SLSASVGDRVTITCRASQDIKNYLNWYQQKPGKAPKLLIYYS |
| | STLLSGVPSRFSGSGSGTDFTLTISSLQPEDFATYYCQQSIT |
| | LPPTFGGGTKVEIKGQPREPQVYTLPPSRDELTKNQVSLSCA |
| | VKGFYPSDIAVEWESNGQPENNYKTTPPVLDSDGSFFLVSKL |
| | TVDKSRWQQGNVFSCSVMHEALHNHYTQKSLSLSPGKGGGGS |
| | GGGGSGGGGSHKSDITLQEIIKTLNSLTEQK |
| SEQ ID NO: 13 | [CD38-VH_CH1_Dig-VH_CH3-Knob] |
| | EVQLLESGGGLVQPGGSLRLSCAVSGFTFNSFAMSWVRQAPG |
| | KGLEWVSAISGSGGGTYYADSVKGRFTISRDNSKNTLYLQMN |
| | SLRAEDTAVYFCAKDKILWFGEPVFDYWGQGTLVTVSSASTK |
| | GPSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCGGGGSGGGGSGGGGSGGGGSQVQLVES |
| | GGGLVKPGGSLRLSCAASGFTFSDYAMSWIRQAPGKGLEWVS |
| | SINIGATYIYYADSVKGRFTISRDNAKNSLYLQMNSLRAEDT |
| | AVYYCARPGSPYEYDKAYYSMAYWGQGTTVTVSSGQPREPQV |
| | YTLPPSRDELTKNQVSLWCLVKGFYPSDIAVEWESNGQPENN |
| | YKTTPPVLDSDGSFFLYSKLTVDKSRWQQGNVFSCSVMHEAL |
| | HNHYTQKSLSLSPGK |
| SEQ ID NO: 14 | [CD38-VH_CH1_Dig-VH_CH3-Knob]-[Linker]-[Split |
| | interleukin-4 (BC)] |
| | EVQLLESGGGLVQPGGSLRLSCAVSGFTFNSFAMSWVRQAPG |
| | KGLEWVSAISGSGGGTYYADSVKGRFTISRDNSKNTLYLQMN |
| | SLRAEDTAVYFCAKDKILWFGEPVFDYWGQGTLVTVSSASTK |
| | GPSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCGGGGSGGGGSGGGGSGGGGSQVQLVES |
| | GGGLVKPGGSLRLSCAASGFTFSDYAMSWIRQAPGKGLEWVS |
| | SINIGATYIYYADSVKGRFTISRDNAKNSLYLQMNSLRAEDT |
| | AVYYCARPGSPYEYDKAYYSMAYWGQGTTVTVSSGQPREPQV |
| | YTLPPSRDELTKNQVSLWCLVKGFYPSDIAVEWESNGQPENN |
| | YKTTPPVLDSDGSFFLYSKLTVDKSRWQQGNVFSCSVMHEAL |
| | HNHYTQKSLSLSPGKGGGGSGGGGSGGGGSTLCTELTVTDIF |
| | AASKNTTEKETFCRAATVLRQFYSHHEKDTRCLGATAQQFHR |
| | HKQLIRFLKRLDRNLWGLAGLNSCP |
| SEQ ID NO: 15 | [Biotin-VL_CH3-Hole-E357K]-[Linker]-[Split interleukin-4 |
| | (DA) E9Q] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSVKEANQSTLENFLERLKTIMREK |
| | YSKCSSGGGSGGSHKCDITLQQIIKTLNSLTEQK |
| SEQ ID NO: 16 | [CD38-VH_CH1_Dig-VL_CH3-Hole]-[Linker]-[Split |
| | interleukin-4 (DA)] |
| | EVQLLESGGGLVQPGGSLRLSCAVSGFTFNSFAMSWVRQAPG |
| | KGLEWVSAISGSGGGTYYADSVKGRFTISRDNSKNTLYLQMN |
| | SLRAEDTAVYFCAKDKILWFGEPVFDYWGQGTLVTVSSASTK |
| | GPSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCGGGGSGGGGSGGGGSGGGGSDIQMTQS |
| | PSSLSASVGDRVTITCRASQDIKNYLNWYQQKPGKAPKLLIY |
| | YSSTLLSGVPSRFSGSGSGTDFTLTISSLQPEDFATYYCQQS |
| | ITLPPTFGGGTKVEIKGQPREPQVYTLPPSRDELTKNQVSLS |
| | CAVKGFYPSDIAVEWESNGQPENNYKTTPPVLDSDGSFFLVS |
| | KLTVDKSRWQQGNVFSCSVMHEALHNHYTQKSLSLSPGKGGG |
| | GSGGGGSGGGGSVKEANQSTLENFLERLKTIMREKYSKCSSG |
| | GGSGGSHKCDITLQEIIKTLNSLTEQK |
| SEQ ID NO: 17 | [Biotin-VH_CH3-Knob-K370E]-[Linker]-[Split interleukin-4 |
| | (BC) R88Q] |
| | QVQLVQSGAEVKKPGSSVKVSCKSSGFNNKDTFFQWVRQAPG |
| | QGLEWMGRIDPANGFTKYAQKFQGRVTITADTSTSTAYMELS |
| | SLRSEDTAVYYCARWDTYGAAWFAYWGQGTLVTVSSGQPREP |
| | QVYTLPPSRDELTKNQVSLWCLVEGFYPSDIAVEWESNGQPE |
| | NNYKTTPPVLDSDGSFFLYSKLTVDKSRWQQGNVFSCSVMHE |
| | ALHNHYTQKSLSLSPGKGGGGSGGGGSGGGGSTLCTELTVTD |
| | IFAASKNTTEKETFCRAATVLRQFYSHHEKDTRCLGATAQQF |
| | HRHKQLIRFLKRLDQNLWGLAGLNSCP |
| SEQ ID NO: 18 | [Biotin-VL_CH3-Hole-E357K]-[Linker]-[Split interleukin-4 |
| | (A)] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSHKSDITLQEIIKTLNSLTEQK |
| SEQ ID NO: 19 | [Biotin-VL_CH3-Hole-E357K]-[Linker]-[Split interleukin-4 |
| | (A) E9A] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSHKSDITLQAIIKTLNSLTEQK |
| SEQ ID NO: 20 | [Biotin-VL_CH3-Hole-E357K]-[Linker]-[Split interleukin-4 |
| | (A) 15A E9Q] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSHKSDATLQQIIKTLNSLTEQK |
| SEQ ID NO: 21 | [Biotin-VL_CH3-Hole-E357K]-[Linker]-[Split interleukin-4 |
| | (A) T6D E9Q] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSHKSDIDLQQIIKTLNSLTEQK |
| SEQ ID NO: 22 | [Biotin-VL_CH3-Hole-E357K]-[Linker]-[Split interleukin-4 |
| | (A) T6D E9A] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSHKSDIDLQAIIKTLNSLTEQK |
| SEQ ID NO: 23 | [Biotin-VH_CH3-Knob-K370E]-[Linker]-[Split interleukin-4 |
| | (BCD) R88A] |
| | QVQLVQSGAEVKKPGSSVKVSCKSSGFNNKDTFFQWVRQAPG |
| | QGLEWMGRIDPANGFTKYAQKFQGRVTITADTSTSTAYMELS |
| | SLRSEDTAVYYCARWDTYGAAWFAYWGQGTLVTVSSGQPREP |
| | QVYTLPPSRDELTKNQVSLWCLVEGFYPSDIAVEWESNGQPE |
| | NNYKTTPPVLDSDGSFFLYSKLTVDKSRWQQGNVFSCSVMHE |
| | ALHNHYTQKSLSLSPGKGGGGSGGGGSGGGGSTLCTELTVTD |
| | IFAASKNTTEKETFCRAATVLRQFYSHHEKDTRCLGATAQQF |
| | HRHKQLIRFLKRLDANLWGLAGLNSCPVKEANQSTLENFLER |
| | LKTIMREKYSKSSS |
| SEQ ID NO: 24 | [Biotin-VH_CH3-Knob-K370E]-[Linker]-[Split interleukin-4 |
| | (BCD) R81E R88Q] |
| | QVQLVQSGAEVKKPGSSVKVSCKSSGFNNKDTFFQWVRQAPG |
| | QGLEWMGRIDPANGFTKYAQKFQGRVTITADTSTSTAYMELS |
| | SLRSEDTAVYYCARWDTYGAAWFAYWGQGTLVTVSSGQPREP |
| | QVYTLPPSRDELTKNQVSLWCLVEGFYPSDIAVEWESNGQPE |
| | NNYKTTPPVLDSDGSFFLYSKLTVDKSRWQQGNVFSCSVMHE |
| | ALHNHYTQKSLSLSPGKGGGGSGGGGSGGGGSTLCTELTVTD |
| | IFAASKNTTEKETFCRAATVLRQFYSHHEKDTRCLGATAQQF |
| | HRHKQLIEFLKRLDQNLWGLAGLNSCPVKEANQSTLENFLER |
| | LKTIMREKYSKSSS |
| SEQ ID NO: 25 | [Biotin-VH_CH3-Knob-K370E]-[Linker]-[Split interleukin-4 |
| | (BCD) K84E R88Q] |
| | QVQLVQSGAEVKKPGSSVKVSCKSSGFNNKDTFFQWVRQAPG |
| | QGLEWMGRIDPANGFTKYAQKFQGRVTITADTSTSTAYMELS |
| | SLRSEDTAVYYCARWDTYGAAWFAYWGQGTLVTVSSGQPREP |
| | QVYTLPPSRDELTKNQVSLWCLVEGFYPSDIAVEWESNGQPE |
| | NNYKTTPPVLDSDGSFFLYSKLTVDKSRWQQGNVFSCSVMHE |
| | ALHNHYTQKSLSLSPGKGGGGSGGGGSGGGGSTLCTELTVTD |
| | IFAASKNTTEKETFCRAATVLRQFYSHHEKDTRCLGATAQQF |
| | HRHKQLIRFLERLDQNLWGLAGLNSCPVKEANQSTLENFLER |
| | LKTIMREKYSKSSS |
| SEQ ID NO: 26 | [Biotin-VH_CH3-Knob-K370E]-[Linker]-[Split interleukin-4 |
| | (BCD) R88Q N89A] |
| | QVQLVQSGAEVKKPGSSVKVSCKSSGFNNKDTFFQWVRQAPG |
| | QGLEWMGRIDPANGFTKYAQKFQGRVTITADTSTSTAYMELS |
| | SLRSEDTAVYYCARWDTYGAAWFAYWGQGTLVTVSSGQPREP |
| | QVYTLPPSRDELTKNQVSLWCLVEGFYPSDIAVEWESNGQPE |
| | NNYKTTPPVLDSDGSFFLYSKLTVDKSRWQQGNVFSCSVMHE |
| | ALHNHYTQKSLSLSPGKGGGGSGGGGSGGGGSTLCTELTVTD |
| | IFAASKNTTEKETFCRAATVLRQFYSHHEKDTRCLGATAQQF |
| | HRHKQLIRFLKRLDQALWGLAGLNSCPVKEANQSTLENFLER |
| | LKTIMREKYSKSSS |
| SEQ ID NO: 27 | [Biotin-VH_CH3-Knob-K370E]-[Linker]-[Split interleukin-4 |
| | (BCD) R88Q W91A] |
| | QVQLVQSGAEVKKPGSSVKVSCKSSGFNNKDTFFQWVRQAPG |
| | QGLEWMGRIDPANGFTKYAQKFQGRVTITADTSTSTAYMELS |
| | SLRSEDTAVYYCARWDTYGAAWFAYWGQGTLVTVSSGQPREP |
| | QVYTLPPSRDELTKNQVSLWCLVEGFYPSDIAVEWESNGQPE |
| | NNYKTTPPVLDSDGSFFLYSKLTVDKSRWQQGNVFSCSVMHE |
| | ALHNHYTQKSLSLSPGKGGGGGGGGSGGGGSTLCTELTVTD |
| | IFAASKNTTEKETFCRAATVLRQFYSHHEKDTRCLGATAQQF |
| | HRHKQLIRFLKRLDQNLAGLAGLNSCPVKEANQSTLENFLER |
| | LKTIMREKYSKSSS |
| SEQ ID NO: 28 | [Biotin-VL_CH3-Hole-E357K]-[Linker]-[Split interleukin-4 |
| | (DA) T6D E9A] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSVKEANQSTLENFLERLKTIMREK |
| | YSKCSSGGGSGGSHKCDIDLQAIIKTLNSLTEQK |
| SEQ ID NO: 29 | [Biotin-VH_CH3-Knob-K370E]-[Linker]-[Split interleukin-4 |
| | (BC) R81E R88Q] |
| | QVQLVQSGAEVKKPGSSVKVSCKSSGFNNKDTFFQWVRQAPG |
| | QGLEWMGRIDPANGFTKYAQKFQGRVTITADTSTSTAYMELS |
| | SLRSEDTAVYYCARWDTYGAAWFAYWGQGTLVTVSSGQPREP |
| | QVYTLPPSRDELTKNQVSLWCLVEGFYPSDIAVEWESNGQPE |
| | NNYKTTPPVLDSDGSFFLYSKLTVDKSRWQQGNVFSCSVMHE |
| | ALHNHYTQKSLSLSPGKGGGGSGGGGSGGGGSTLCTELTVTD |
| | IFAASKNTTEKETFCRAATVLRQFYSHHEKDTRCLGATAQQF |
| | HRHKQLIEFLKRLDQNLWGLAGLNSCP |
| SEQ ID NO: 30 | [Her2-VH_CH1_Dig-VH_CH3-Knob]-[Linker]-[Split |
| | interleukin-4 (BC)] |
| | EVQLVESGGGLVQPGGSLRLSCAASGFNIKDTYIHWVRQAPG |
| | KGLEWVARIYPTNGYTRYADSVKGRFTISADTSKNTAYLQMN |
| | SLRAEDTAVYYCSRWGGDGFYAMDYWGQGTLVTVSSASTKGP |
| | SVFPLAPSSKSTSGGTAALGCLVKDYFPEPVTVSWNSGALTS |
| | GVHTFPAVLQSSGLYSLSSVVTVPSSSLGTQTYICNVNHKPS |
| | NTKVDKKVEPKSCGGGGSGGGGSGGGGSGGGGSQVQLVESGG |
| | GLVKPGGSLRLSCAASGFTFSDYAMSWIRQAPGKGLEWVSSI |
| | NIGATYIYYADSVKGRETISRDNAKNSLYLQMNSLRAEDTAV |
| | YYCARPGSPYEYDKAYYSMAYWGQGTTVTVSSGQPREPQVYT |
| | LPPSRDELTKNQVSLWCLVKGFYPSDIAVEWESNGQPENNYK |
| | TTPPVLDSDGSFFLYSKLTVDKSRWQQGNVFSCSVMHEALHN |
| | HYTQKSLSLSPGKGGGGSGGGGSGGGGSTLCTELTVTDIFAA |
| | SKNTTEKETFCRAATVLRQFYSHHEKDTRCLGATAQQFHRHK |
| | QLIRFLKRLDRNLWGLAGLNSCP |
| SEQ ID NO: 31 | [Her2-VH_CH1_Dig-VL_CH3-Hole]-[Linker]-[Split |
| | interleukin-4 (DA)] |
| | EVQLVESGGGLVQPGGSLRLSCAASGFNIKDTYIHWVRQAPG |
| | KGLEWVARIYPTNGYTRYADSVKGRFTISADTSKNTAYLQMN |
| | SLRAEDTAVYYCSRWGGDGFYAMDYWGQGTLVTVSSASTKGP |
| | SVFPLAPSSKSTSGGTAALGCLVKDYFPEPVTVSWNSGALTS |
| | GVHTFPAVLQSSGLYSLSSVVTVPSSSLGTQTYICNVNHKPS |
| | NTKVDKKVEPKSCGGGGSGGGGSGGGGSGGGGSDIQMTQSPS |
| | SLSASVGDRVTITCRASQDIKNYLNWYQQKPGKAPKLLIYYS |
| | STLLSGVPSRFSGSGSGTDFTLTISSLQPEDFATYYCQQSIT |
| | LPPTFGGGTKVEIKGQPREPQVYTLPPSRDELTKNQVSLSCA |
| | VKGFYPSDIAVEWESNGQPENNYKTTPPVLDSDGSFFLVSKL |
| | TVDKSRWQQGNVFSCSVMHEALHNHYTQKSLSLSPGKGGGGS |
| | GGGGSGGGGSVKEANQSTLENFLERLKTIMREKYSKCSSGGG |
| | SGGSHKCDITLQEIIKTLNSLTEQK |
| SEQ ID NO: 32 | [LeY-VH_CH1_Dig-VL_CH3-Hole] |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGGGSDIQMTQSPSS |
| | LSASVGDRVTITCRASQDIKNYLNWYQQKPGKAPKLLIYYSS |
| | TLLSGVPSRFSGSGSGTDFTLTISSLQPEDFATYYCQQSITL |
| | PPTFGGGTKVEIKGQPREPQVYTLPPSRDELTKNQVSLSCAV |
| | KGFYPSDIAVEWESNGQPENNYKTTPPVLDSDGSFFLVSKLT |
| | VDKSRWQQGNVFSCSVMHEALHNHYTQKSLSLSPGK |
| SEQ ID NO: 33 | [Biotin-VH_CH3-Knob-K370E] |
| | QVQLVQSGAEVKKPGSSVKVSCKSSGFNNKDTFFQWVRQAPG |
| | QGLEWMGRIDPANGFTKYAQKFQGRVTITADTSTSTAYMELS |
| | SLRSEDTAVYYCARWDTYGAAWFAYWGQGTLVTVSSGQPREP |
| | QVYTLPPSRDELTKNQVSLWCLVEGFYPSDIAVEWESNGQPE |
| | NNYKTTPPVLDSDGSFFLYSKLTVDKSRWQQGNVFSCSVMHE |
| | ALHNHYTQKSLSLSPGK |
| SEQ ID NO: 34 | [LeY-VH_CH1_Dig-VH_CH3-Knob]-[Linker]-[Interleukin- |
| | 2v] |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGGGSQVQLVESGGG |
| | LVKPGGSLRLSCAASGFTFSDYAMSWIRQAPGKGLEWVSSIN |
| | IGATYIYYADSVKGRFTISRDNAKNSLYLQMNSLRAEDTAVY |
| | YCARPGSPYEYDKAYYSMAYWGQGTTVTVSSGQPREPQVYTL |
| | PPSRDELTKNQVSLWCLVKGFYPSDIAVEWESNGQPENNYKT |
| | TPPVLDSDGSFFLYSKLTVDKSRWQQGNVFSCSVMHEALHNH |
| | YTQKSLSLSPGKGGGGSGGGGSGGGGSAPASSSTKKTQLQLE |
| | HLLLDLQMILNGINNYSKNPKLTRMLTAKFAMPKKATELKHL |
| | QCLEEELKPLEEVLNGAQSKNFHLRPRDLISNINVIVLELKG |
| | SETTFMCEYADETATIVEFLNRWITFSQSIISTLT |
| SEQ ID NO: 35 | [Biotin-VL_CH3-Hole-E357K]-[Linker]-[IL-2RB extracellular |
| | domain (A27 - D239)] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSAVNGTSQFTCFYNSRANISCVWS |
| | QDGALQDTSCQVHAWPDRRRWNQTCELLPVSQASWACNLILG |
| | APDSQKLTTVDIVTLRVLCREGVRWRVMAIQDEKPFENLRLM |
| | APISLQVVHVETHRCNISWEISQASHYFERHLEFEARTLSPG |
| | HTWEEAPLLTLKQKQEWICLETLTPDTQYEFQVRVKPLQGEF |
| | TTWSPWSQPLAFRTKPAALGKD |
| SEQ ID NO: 36 | [Biotin-VL_CH3-Hole-E357K]-[Linker]-[Common |
| | gamma |
| | chain (yc) extracellular domain (L23 - N254)] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSLNTTILTPNGNEDTTADFFLTTM |
| | PTDSLSVSTLPLPEVQCEVENVEYMNCTWNSSSEPQPTNLTL |
| | HYWYKNSDNDKVQKCSHYLFSEEITSGCQLQKKEIHLYQTFV |
| | VQLQDPREPRRQATQMLKLQNLVIPWAPENLTLHKLSESQLE |
| | LNWNNRFLNHCLEHLVQYRTDWDHSWTEQSVDYRHKFSLPSV |
| | DGQKRYTFRVRSRFNPLCGSAQHWSEWSHPIHWGSNTSKEN |
| SEQ ID NO: 37 | [Biotin-VL_CH3-Hole-E357K]-[Linker]-[IL-2RB_yc] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGSGGGGSGGGGSAVNGTSQFTCFYNSRANISCVWS |
| | QDGALQDTSCQVHAWPDRRRWNQTCELLPVSQASWACNLILG |
| | APDSQKLTTVDIVTLRVLCREGVRWRVMAIQDEKPFENLRLM |
| | APISLQVVHVETHRCNISWEISQASHYFERHLEFEARTLSPG |
| | HTWEEAPLLTLKQKQEWICLETLTPDTQYEFQVRVKPLQGEF |
| | TTWSPWSQPLAFRTKPAALGKDGGGGSGGGGSGGGGSLNTTI |
| | LTPNGNEDTTADFFLTTMPTDSLSVSTLPLPEVQCEVENVEY |
| | MNCTWNSSSEPQPTNLTLHYWYKNSDNDKVQKCSHYLESEEI |
| | TSGCQLQKKEIHLYQTFVVQLQDPREPRRQATQMLKLQNLVI |
| | PWAPENLTLHKLSESQLELNWNNRFLNHCLEHLVQYRTDWDH |
| | SWTEQSVDYRHKFSLPSVDGQKRYTFRVRSRENPLCGSAQHW |
| | SEWSHPIHWGSNTSKEN |
| SEQ ID NO: 38 | [CD38-VH_CH1_Dig-VH_CH3-Knob]-[Linker]-[IL-12 p35 |
| | (C73S)] |
| | EVQLLESGGGLVQPGGSLRLSCAVSGFTFNSFAMSWVRQAPG |
| | KGLEWVSAISGSGGGTYYADSVKGRFTISRDNSKNTLYLQMN |
| | SLRAEDTAVYFCAKDKILWFGEPVFDYWGQGTLVTVSSASTK |
| | GPSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCGGGGSGGGGSGGGGSGGGGSQVQLVES |
| | GGGLVKPGGSLRLSCAASGFTFSDYAMSWIRQAPGKGLEWVS |
| | SINIGATYIYYADSVKGRFTISRDNAKNSLYLQMNSLRAEDT |
| | AVYYCARPGSPYEYDKAYYSMAYWGQGTTVTVSSGQPREPQV |
| | YTLPPSRDELTKNQVSLWCLVKGFYPSDIAVEWESNGQPENN |
| | YKTTPPVLDSDGSFFLYSKLTVDKSRWQQGNVFSCSVMHEAL |
| | HNHYTQKSLSLSPGKGGGGSGGGGSGGGGSRNLPVATPDPGM |
| | FPCLHHSQNLLRAVSNMLQKARQTLEFYPCTSEEIDHEDITK |
| | DETSTVEACLPLELTKNESSLNSRETSFITNGSCLASRKTSF |
| | MMALCLSSIYEDLKMYQVE FKTMNAKLLMDPKRQIFLDQNML |
| | AVIDELMQALNENSETVPQKSSLEEPDFYKTKIKLCILLHAF |
| | RIRAVTIDRVMSYLNAS |
| SEQ ID NO: 39 | [LeY-VH_CH1_Dig-VL_CH3-Hole]-[Linker]-[IL-12 p40 |
| | (C177S)] |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGGGSDIQMTQSPSS |
| | LSASVGDRVTITCRASQDIKNYLNWYQQKPGKAPKLLIYYSS |
| | TLLSGVPSRFSGSGSGTDFTLTISSLQPEDFATYYCQQSITL |
| | PPTFGGGTKVEIKGQPREPQVYTLPPSRDELTKNQVSLSCAV |
| | KGFYPSDIAVEWESNGQPENNYKTTPPVLDSDGSFFLVSKLT |
| | VDKSRWQQGNVFSCSVMHEALHNHYTQKSLSLSPGKGGGGSG |
| | GGGSGGGGSIWELKKDVYVVELDWYPDAPGEMVVLTCDTPEE |
| | DGITWTLDQSSEVLGSGKTLTIQVKEFGDAGQYTCHKGGEVL |
| | SHSLLLLHKKEDGIWSTDILKDQKEPKNKTFLRCEAKNYSGR |
| | FTCWWLTTISTDLTFSVKSSRGSSDPQGVTCGAATLSAERVR |
| | GDNKEYEYSVECQEDSASPAAEESLPIEVMVDAVHKLKYENY |
| | TSSFFIRDIIKPDPPKNLQLKPLKNSRQVEVSWEYPDTWSTP |
| | HSYFSLTFCVQVQGKSKREKKDRVFTDKTSATVICRKNASIS |
| | VRAQDRYYSSSWSEWASVPCS |
| SEQ ID NO: 40 | [LeY-VH_CH1_Dig-VH_CH3-Knob]-[Linker]-[IL-12 p35 |
| | (C73S)] |
| | DVKLVESGGGLVQPGGSLKLSCATSGFTFSDYYMYWVRQTPE |
| | KRLEWVAYISNDDSSAAYSDTVKGRFTISRDNARNTLYLQMS |
| | RLKSEDTAIYYCARGLAWGAWFAYWGQGTLVTVSSASTKGPS |
| | VFPLAPSSKSTSGGTAALGCLVKDYFPEPVTVSWNSGALTSG |
| | VHTFPAVLQSSGLYSLSSVVTVPSSSLGTQTYICNVNHKPSN |
| | TKVDKKVEPKSCGGGGSGGGGSGGGGSGGGGSQVQLVESGGG |
| | LVKPGGSLRLSCAASGFTFSDYAMSWIRQAPGKGLEWVSSIN |
| | IGATYIYYADSVKGRFTISRDNAKNSLYLQMNSLRAEDTAVY |
| | YCARPGSPYEYDKAYYSMAYWGQGTTVTVSSGQPREPQVYTL |
| | PPSRDELTKNQVSLWCLVKGFYPSDIAVEWESNGQPENNYKT |
| | TPPVLDSDGSFFLYSKLTVDKSRWQQGNVFSCSVMHEALHNH |
| | YTQKSLSLSPGKGGGGSGGGGSGGGGSRNLPVATPDPGMFPC |
| | LHHSQNLLRAVSNMLQKARQTLEFYPCTSEEIDHEDITKDKT |
| | STVEACLPLELTKNESSLNSRETSFITNGSCLASRKTSEMMA |
| | LCLSSIYEDLKMYQVEFKTMNAKLLMDPKRQIFLDQNMLAVI |
| | DELMQALNENSETVPQKSSLEEPDFYKTKIKLCILLHAFRIR |
| | AVTIDRVMSYLNAS |
| SEQ ID NO: 41 | [Biotin-VL_CH3-Hole-E357K]-[Linker]-[IL-12 p40i (C177S)] |
| | DIQMTQSPSSLSASVGDRVTITCRASGNIHNYLSWYQQKPGK |
| | VPKLLIYSAKTLADGVPSRFSGSGSGTDFTLTISSLQPEDVA |
| | TYYCQHFWSSIYTFGQGTKLEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGKGGGGGGGGSGGGGSSTDILKDQKEPKNKTFLRCEAKN |
| | YSGRFTCWWLTTISTDLTFSVKSSRGSSDPQGVTCGAATLSA |
| | ERVRGDNKEYEYSVECQEDSASPAAEESLPIEVMVDAVHKLK |
| | YENYTSSFFIRDIIKPDPPKNLQLKPLKNSRQVEVSWEYPDT |
| | WSTPHSYFSLTFCVQVQGKSKREKKDRVFTDKTSATVICRKN |
| | ASISVRAQDRYYSSSWSEWASVPCS |
| SEQ ID NO: 42 | [CD38-VH_CH1_Dig-VH_CH3-Knob]-[Linker]-[SmBiT] |
| | EVQLLESGGGLVQPGGSLRLSCAVSGFTFNSFAMSWVRQAPG |
| | KGLEWVSAISGSGGGTYYADSVKGRFTISRDNSKNTLYLQMN |
| | SLRAEDTAVYFCAKDKILWFGEPVFDYWGQGTLVTVSSASTK |
| | GPSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCGGGGSGGGGSGGGGSGGGGSQVQLVES |
| | GGGLVKPGGSLRLSCAASGFTFSDYAMSWIRQAPGKGLEWVS |
| | SINIGATYIYYADSVKGRFTISRDNAKNSLYLQMNSLRAEDT |
| | AVYYCARPGSPYEYDKAYYSMAYWGQGTTVTVSSGQPREPQV |
| | YTLPPSRDELTKNQVSLWCLVKGFYPSDIAVEWESNGQPENN |
| | YKTTPPVLDSDGSFFLYSKLTVDKSRWQQGNVFSCSVMHEAL |
| | HNHYTQKSLSLSPGKGGGGSGGGGSGGGGSVTGYRLFEEIL |
| SEQ ID NO: 43 | [NADA-VL_CH3-Hole-E357K] |
| | DIQMTQSPSSLSASVGDRVTITCRASQDVGSGVAWYQQKPGK |
| | APKLLIGSASGLYSGVPSRFSGSRSGTDFTLTISSLQPEDFA |
| | TYYCQQSASGGSTFGQGTKVEIKGQPREPQVYTLPPSRDKLT |
| | KNQVSLSCAVKGFYPSDIAVEWESNGQPENNYKTTPPVLDSD |
| | GSFFLVSKLTVDKSRWQQGNVFSCSVMHEALHNHYTQKSLSL |
| | SPGK |
| SEQ ID NO: 44 | [CD38-VH_CH1_Dig-VL_CH3-Hole]-[Linker]-[LgBiT] |
| | EVQLLESGGGLVQPGGSLRLSCAVSGFTFNSFAMSWVRQAPG |
| | KGLEWVSAISGSGGGTYYADSVKGRFTISRDNSKNTLYLQMN |
| | SLRAEDTAVYFCAKDKILWFGEPVFDYWGQGTLVTVSSASTK |
| | GPSVFPLAPSSKSTSGGTAALGCLVKDYFPEPVTVSWNSGAL |
| | TSGVHTFPAVLQSSGLYSLSSVVTVPSSSLGTQTYICNVNHK |
| | PSNTKVDKKVEPKSCGGGGSGGGGSGGGGSGGGGSDIQMTQS |
| | PSSLSASVGDRVTITCRASQDIKNYLNWYQQKPGKAPKLLIY |
| | YSSTLLSGVPSRFSGSGSGTDFTLTISSLQPEDFATYYCQQS |
| | ITLPPTFGGGTKVEIKGQPREPQVYTLPPSRDELTKNQVSLS |
| | CAVKGFYPSDIAVEWESNGQPENNYKTTPPVLDSDGSFFLVS |
| | KLTVDKSRWQQGNVFSCSVMHEALHNHYTQKSLSLSPGKGGG |
| | GSGGGGSGGGGSMVFTLEDFVGDWEQTAAYNLDQVLEQGGVS |
| | SLLQNLAVSVTPIQRIVRSGFNALKIDIHVIIPYEGLSADQM |
| | AQIEEVFKVVYPVDDHHFKVILPYGTLVIDGVTPNMLNYEGR |
| | PYEGIAVFDGKKITVTGTLWNGNKIIDERLITPDGSMLFRVT |
| | INS |
| SEQ ID NO: 45 | [NADA-VH_CH3-Knob-K370E] |
| | EVQLVESGGGLVQPGGSLRLSCAASGESIAGTAIHWVRQAPG |
| | KGLEWVASISPGGGSTAYADSVKGRFTISADTSKNTAYLQMN |
| | SLRAEDTAVYYCSRSGGSGASAMDYWGQGTLVTVSSGQPREP |
| | QVYTLPPSRDELTKNQVSLWCLVEGFYPSDIAVEWESNGQPE |
| | NNYKTTPPVLDSDGSFFLYSKLTVDKSRWQQGNVFSCSVMHE |
| | ALHNHYTQKSLSLSPGK |

### EXAMPLES

The following examples are provided to aid the understanding of the present invention, the true scope of which is set forth in the appended claims. It is understood that modifications can be made in the procedures set forth without departing from the spirit of the invention.

The arrangement of the molecules used in the Examples are indicated in the following Table:

| SEQ | | | | |
|---|---|---|---|---|
| ID NO: | chain name | subunit 1 | subunit 2 | subunit 3 |
| 1 | [LeY-VH_CH1_Dig- | 1: | | |
| | VH_CH3-Knob] | LeY- | | |
| | | VH_CH1_Dig- | | |
| | | VH_CH3- | | |
| | | Knob | | |
| 2 | [LeY-VL_Ckappa] | 2: | | |
| | | LeY- | | |
| | | VL_Ckappa | | |
| 3 | [Dig-VL_CH3-Hole]- | 3: | 4: | 5: |
| | [Linker]-[Human | Dig-VL_CH3- | Linker | Human |
| | interleukin-4] | Hole | | interleukin-4 |
| 4 | [Dig-VL_CH3-Hole]- | 3: | 4: | 6: |
| | [Linker]-[Interleukin-4 | Dig-VL_CH3- | Linker | Interleukin-4 |
| | DABC (circularly | Hole | | DABC (circularly |
| | permutated)] | | | permutated) |
| 5 | [CD38-VH_CH1_Dig- | 7: | 4: | 8: |
| | VH_CH3-Knob]- | CD38- | Linker | Split interleukin-4 |
| | [Linker]-[Split | VH_CH1_Dig- | | (BCD) |
| | interleukin-4 (BCD)] | VH_CH3- | | |
| | | Knob | | |
| 6 | [CD38-VL_Ckappa] | 9: | | |
| | | CD38- | | |
| | | VL_Ckappa | | |
| 7 | [Biotin-VL_CH3-Hole- | 10: | 4: | 11: |
| | E357K]-[Linker]-[Split | Biotin- | Linker | Split interleukin-4 |
| | interleukin-4 (A) E9Q] | VL_CH3- | | (A) E9Q |
| | | Hole-E357K | | |
| 8 | [CD38-VH_CH1_Dig- | 12: | 4: | 13: |
| | VL_CH3-Hole]- | CD38- | Linker | Split interleukin-4 |
| | [Linker]-[Split | VH_CH1_Dig- | | (A) |
| | interleukin-4 (A)] | VL_CH3-Hole | | |
| 9 | [Biotin-VH_CH3-Knob- | 14: | 4: | 15: |
| | K370E]-[Linker]-[Split | Biotin- | Linker | Split interleukin-4 |
| | interleukin-4 (BCD) | VH_CH3- | | (BCD) R88Q |
| | R88Q] | Knob-K370E | | |
| 10 | [Her2-VH_CH1_Dig- | 16: | 4: | 8: |
| | VH_CH3-Knob]- | Her2- | Linker | Split interleukin-4 |
| | [Linker]-[Split | VH_CH1_Dig- | | (BCD) |
| | interleukin-4 (BCD)] | VH_CH3- | | |
| | | Knob | | |
| 11 | [Her2-VL_Ckappa] | 17: | | |
| | | Her2- | | |
| | | VL_Ckappa | | |
| 12 | [Her2-VH_CH1_Dig- | 18: | 4: | 13: |
| | VL_CH3-Hole]- | Her2- | Linker | Split interleukin-4 |
| | [Linker]-[Split | VH_CH1_Dig- | | (A) |
| | interleukin-4 (A)] | VL_CH3-Hole | | |
| 13 | [CD38-VH_CH1_Dig- | 7: | | |
| | VH_CH3-Knob] | CD38- | | |
| | | VH_CH1_Dig- | | |
| | | VH_CH3- | | |
| | | Knob | | |
| 14 | [CD38-VH_CH1_Dig- | 7: | 4: | 19: |
| | VH_CH3-Knob]- | CD38- | Linker | Split interleukin-4 |
| | [Linker]-[Split | VH_CH1_Dig- | | (BC) |
| | interleukin-4 (BC)] | VH_CH3- | | |
| | | Knob | | |
| 15 | [Biotin-VL_CH3-Hole- | 10: | 4: | 20: |
| | E357K]-[Linker]-[Split | Biotin- | Linker | Split interleukin-4 |
| | interleukin-4 (DA) E9Q] | VL_CH3- | | (DA) E9Q |
| | | Hole-E357K | | |
| 16 | [CD38-VH_CH1_Dig- | 12: | 4: | 21: |
| | VL_CH3-Hole]- | CD38- | Linker | Split interleukin-4 |
| | [Linker]-[Split | VH_CH1_Dig- | | (DA) |
| | interleukin-4 (DA)] | VL_CH3-Hole | | |
| 17 | [Biotin-VH_CH3-Knob- | 14: | 4: | 22: |
| | K370E]-[Linker]-[Split | Biotin- | Linker | Split interleukin-4 |
| | interleukin-4 (BC) R88Q] | VH_CH3- | | (BC) R88Q |
| | | Knob-K370E | | |
| 18 | [Biotin-VL_CH3-Hole- | 10: | 4: | 13: |
| | E357K]-[Linker]-[Split | Biotin- | Linker | Split interleukin-4 |
| | interleukin-4 (A)] | VL_CH3- | | (A) |
| | | Hole-E357K | | |
| 19 | [Biotin-VL_CH3-Hole- | 10: | 4: | 23: |
| | E357K]-[Linker]-[Split | Biotin- | Linker | Split interleukin-4 |
| | interleukin-4 (A) E9A] | VL_CH3- | | (A) E9A |
| | | Hole-E357K | | |
| 20 | [Biotin-VL_CH3-Hole- | 10: | 4: | 24: |
| | E357K]-[Linker]-[Split | Biotin- | Linker | Split interleukin-4 |
| | interleukin-4 (A) I5A | VL_CH3- | | (A) I5A E9Q |
| | E9Q] | Hole-E357K | | |
| 21 | [Biotin-VL_CH3-Hole- | 10: | 4: | 25: |
| | E357K]-[Linker]-[Split | Biotin- | Linker | Split interleukin-4 |
| | interleukin-4 (A) T6D | VL_CH3- | | (A) T6D E9Q |
| | E9Q] | Hole-E357K | | |
| 22 | [Biotin-VL_CH3-Hole- | 10: | 4: | 26: |
| | E357K]-[Linker]-[Split | Biotin- | Linker | Split interleukin-4 |
| | interleukin-4 (A) T6D | VL_CH3- | | (A) T6D E9A |
| | E9A] | Hole-E357K | | |
| 23 | [Biotin-VH_CH3-Knob- | 14: | 4: | 27: |
| | K370E]-[Linker]-[Split | Biotin- | Linker | Split interleukin-4 |
| | interleukin-4 (BCD) | VH_CH3- | | (BCD) R88A |
| | R88A] | Knob-K370E | | |
| 24 | [Biotin-VH_CH3-Knob- | 14: | 4: | 28: |
| | K370E]-[Linker]-[Split | Biotin- | Linker | Split interleukin-4 |
| | interleukin-4 (BCD) | VH_CH3- | | (BCD) R81E |
| | R81E R88Q] | Knob-K370E | | R88Q |
| 25 | [Biotin-VH_CH3-Knob- | 14: | 4: | 29: |
| | K370E]-[Linker]-[Split | Biotin- | Linker | Split interleukin-4 |
| | interleukin-4 (BCD) | VH_CH3- | | (BCD) K84E |
| | K84E R88Q] | Knob-K370E | | R88Q |
| 26 | [Biotin-VH_CH3-Knob- | 14: | 4: | 30: |
| | K370E]-[Linker]-[Split | Biotin- | Linker | Split interleukin-4 |
| | interleukin-4 (BCD) | VH_CH3- | | (BCD) R88Q |
| | R88Q N89A] | Knob-K370E | | N89A |
| 27 | [Biotin-VH_CH3-Knob- | 14: | 4: | 31: |
| | K370E]-[Linker]-[Split | Biotin- | Linker | Split interleukin-4 |
| | interleukin-4 (BCD) | VH_CH3- | | (BCD) R88Q |
| | R88Q W91A] | Knob-K370E | | W91A |
| 28 | [Biotin-VL_CH3-Hole- | 10: | 4: | 32: |
| | E357K]-[Linker]-[Split | Biotin- | Linker | Split interleukin-4 |
| | interleukin-4 (DA) T6D | VL_CH3- | | (DA) T6D E9A |
| | E9A] | Hole-E357K | | |
| 29 | [Biotin-VH_CH3-Knob- | 14: | 4: | 33: |
| | K370E]-[Linker]-[Split | Biotin- | Linker | Split interleukin-4 |
| | interleukin-4 (BC) R81E | VH_CH3- | | (BC) R81E R88Q |
| | R88Q] | Knob-K370E | | |
| 30 | [Her2-VH_CH1_Dig- | 16: | 4: | 19: |
| | VH_CH3-Knob]- | Her2- | Linker | Split interleukin-4 |
| | [Linker]-[Split | VH_CH1_Dig- | | (BC) |
| | interleukin-4 (BC)] | VH_CH3- | | |
| | | Knob | | |
| 31 | [Her2-VH_CH1_Dig- | 18: | 4: | 21: |
| | VL_CH3-Hole]- | Her2- | Linker | Split interleukin-4 |
| | [Linker]-[Split | VH_CH1_Dig- | | (DA) |
| | interleukin-4 (DA)] | VL_CH3-Hole | | |
| 32 | [LeY-VH_CH1_Dig- | 34: | | |
| | VL_CH3-Hole] | LeY- | | |
| | | VH_CH1_Dig- | | |
| | | VL_CH3-Hole | | |
| 33 | [Biotin-VH_CH3-Knob- | 14: | | |
| | K370E] | Biotin- | | |
| | | VH_CH3- | | |
| | | Knob-K370E | | |
| 34 | [LeY-VH_CH1_Dig- | 1: | 4: | 35: |
| | VH_CH3-Knob]- | LeY- | Linker | Interleukin-2v |
| | [Linker]-[Interleukin-2v] | VH_CH1_Dig- | | |
| | | VH_CH3- | | |
| | | Knob | | |
| 35 | [Biotin-VL_CH3-Hole- | 10: | 4: | 36: |
| | E357K]-[Linker]-[IL- | Biotin- | Linker | IL-2Rβ |
| | 2Rβ extracellular domain | VL_CH3- | | extracellular |
| | (A27-D239)] | Hole-E357K | | domain (A27-D239) |
| 36 | [Biotin-VL_CH3-Hole- | 10: | 4: | 37: |
| | E357K]-[Linker]- | Biotin- | Linker | Common gamma |
| | [Common gamma chain | VL_CH3- | | chain (γc) |
| | (γc) extracellular domain | Hole-E357K | | extracellular |
| | (L23-N254)] | | | domain (L23-N254) |
| 37 | [Biotin-VL_CH3-Hole- | 10: | 4: | 38: |
| | E357K]-[Linker]-[IL- | Biotin- | Linker | IL-2Rβ_γc |
| | 2Rβ_γc] | VL_CH3- | | |
| | | Hole-E357K | | |
| 38 | [CD38-VH_CH1_Dig- | 7: | 4: | 39: |
| | VH_CH3-Knob]- | CD38- | Linker | IL-12 p35 (C73S) |
| | [Linker]-[IL-12 p35 | VH_CH1_Dig- | | |
| | (C73S)] | VH_CH3- | | |
| | | Knob | | |
| 39 | [LeY-VH_CH1_Dig- | 34: | 4: | 40: |
| | VL_CH3-Hole]- | LeY- | Linker | IL-12 p40 |
| | [Linker]-[IL-12 p40 | VH_CH1_Dig- | | (C177S) |
| | (C177S)] | VL_CH3-Hole | | |
| 40 | [LeY-VH_CH1_Dig- | 1: | 4: | 39: |
| | VH_CH3-Knob]- | LeY- | Linker | IL-12 p35 (C73S) |
| | [Linker]-[IL-12 p35 | VH_CH1_Dig- | | |
| | (C73S)] | VH_CH3- | | |
| | | Knob | | |
| 41 | [Biotin-VL_CH3-Hole- | 10: | 4: | 41: |
| | E357K]-[Linker]-[IL-12 | Biotin- | Linker | IL-12 p40i |
| | p40i (C177S)] | VL_CH3- | | (C177S) |
| | | Hole-E357K | | |
| 42 | [CD38-VH_CH1_Dig- | 7: | 4: | 42: |
| | VH_CH3-Knob]- | CD38- | Linker | SmBiT |
| | [Linker]-[SmBiT] | VH_CH1_Dig- | | |
| | | VH_CH3- | | |
| | | Knob | | |
| 43 | [NADA-VL_CH3-Hole- | 43: | | |
| | E357K] | NADA- | | |
| | | VL_CH3- | | |
| | | Hole-E357K | | |
| 44 | [CD38-VH_CH1_Dig- | 12: | 4: | 44: |
| | VL_CH3-Hole]- | CD38- | Linker | LgBiT |
| | [Linker]-[LgBiT] | VH_CH1_Dig- | | |
| | | VL_CH3-Hole | | |
| 45 | [NADA-VH_CH3-Knob- | 45: | | |
| | K370E] | NADA- | | |
| | | VH_CH3- | | |
| | | Knob-K370E | | |

The following recombinant proteins are shown in the indicated FIG. (protein names according to Tables 1, 2 and 3 below):

| protein name | FIG. |
|---|---|
| <LeY> IL-4 | 4c, d |
| <LeY> IL-4 DABC | 4c, d |
| <CD38> R1 3 + 1 E9Q | 5, 6, 9 |
| <CD38> R2 3 + 1 R88Q | 5, 6, 9 |
| <Her2> R1 3 + 1 E9Q | 5 |
| <Her2> R2 3 + 1 R88Q | 5 |
| <CD38> IL-4 | 6, 9 |
| <CD38> P1 3 + 1 | 6, 9 |
| <CD38> IL-4 DABC | 6, 9 |
| <CD38> R1 2 + 2 E9Q | 6 |
| <CD38> R2 2 + 2 R88Q | 6 |
| <CD38> P1 2 + 2 | 6, 8, 9 |
| <CD38> R1 3 + 1 no mutation | 7 |
| <CD38> R1 3 + 1 E9A | 7 |
| <CD38> R1 3 + 1 I5A E9Q | 7 |
| <CD38> R1 3 + 1 T6D E9Q | 7 |
| <CD38> R1 3 + 1 T6D E9A | 7, 9 |
| <CD38> R2 3 + 1 R88A | 7 |
| <CD38> R2 3 + 1 R81E R88Q | 7, 9 |
| <CD38> R2 3 + 1 K84E R88Q | 7 |
| <CD38> R2 3 + 1 R88Q N89A | 7 |
| <CD38> R2 3 + 1 R88Q W91A | 7 |
| <CD38> R1 2 + 2 T6D E9A | 8 |
| <CD38> R2 2 + 2 R81E R88Q | 8 |
| <Her2> R1 2 + 2 T6D E9A | 8 |
| <Her2> R2 2 + 2 R81E R88Q | 8 |
| <Her2> P1 2 + 2 | 8 |
| <LeY> R2 | 11 |
| <LeY> P1 IL-2v | 11, 12 |
| <LeY> R1 IL2-Rβ | 11, 12 |
| <LeY> R1 γc | 11, 12 |
| <LeY> R1 IL-2Rβ_γc | 11, 12 |
| <LeY> P1 IL-12 | 14, 15 |
| <LeY> R1 IL-12 p40i | 14, 15 |
| <CD38> R1 SmBiT | 16 |
| <CD38> R2 LgBiT | 16 |

### Example 1

### Design of Activatable Precursor Proteins Comprising Artificially Splitted IL-4

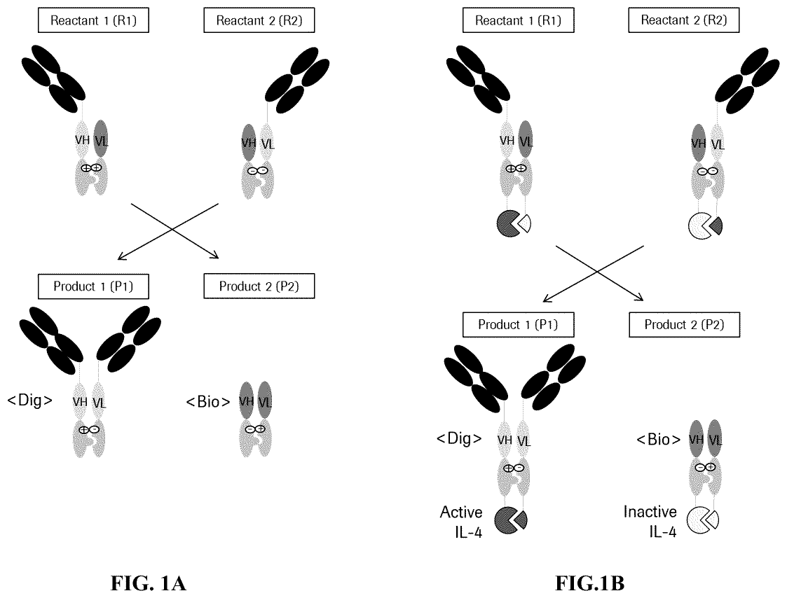

Presursor proteins of the general domain arrangement illustrated in FIG. 1 were prepared. Two precursor proteins (R1 and R2) contain CH3 domains with knob and hole mutations and further destabilizing mutations. The precursor proteins have a half-IgG-like shape, wherein a non-functional pair of VH/VL doamins is arranged at the N-terminal end of the CH3 domains. The precursor proteins further comprise a Fab fragment specifically binding to CD38 or Her2.

Portions of a splitted IL-4 were fused to the C-terminus of each polypeptide as indicated in FIG. 1b. The two precursor proteins comprise cytokine portions that are functionally inactive, but are combinated to an activated IL-4 molcule upon polypeptide chain exchange in one of the product proteins, i.e. the activated protein (FIG. 1b). This was realized by fusing the activated IL-4 portion to the CH3(knob) polypeptide of R1 and the corresponding activated IL-4 portion to the CH3(hole) polypeptide of R2.

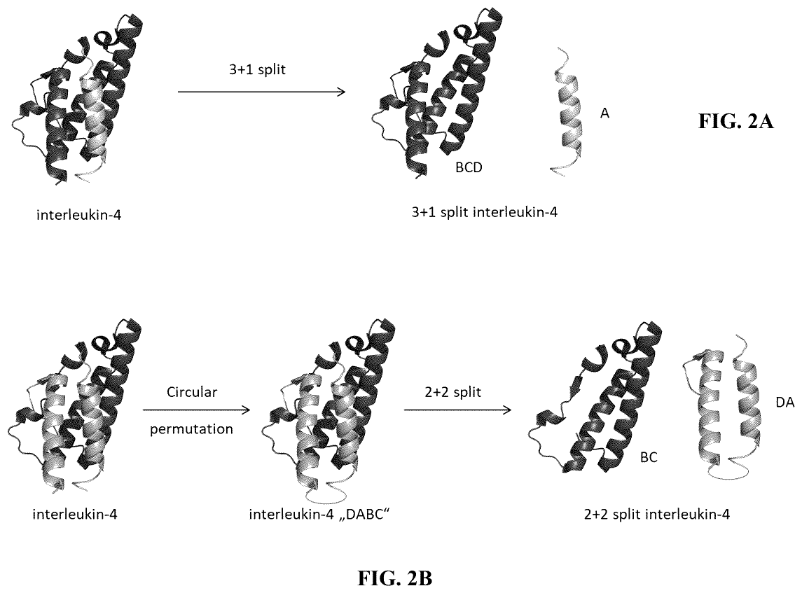

Design of the splitted IL-4 molecule was done as follows: Human interleukin-4 consists of a single polypeptide chain with four alpha-helix domains, herein termed A, B, C and D. Two different split designs of IL-4 were made.
For the “3+1” split design, one disulfide bridge was removed by C3S and C128S mutations. The first helix (residues 1-21) constitutes one part of 3+1 split IL-4 (“A”), while the remaining structure (residues 22-130) constitutes the other part of 3+1 split IL-4 (“BCD”) (FIG. 2a). For the split IL-4 PACE approach, the two split IL-4 units were fused with a flexible linker to the C-terminus the CH3 domains of the precursor proteins. Each molecule (R1, R2, P1, P2) carried one “BCD” unit and one “A” unit.
For the “2+2” split design, IL-4 was circularly permutated by connecting the C- and N-termini of IL-4 with a 7-residue flexible linker, and setting the new C- and N-termini at P100 and V101, respectively. The first two helices constitute one part of 2+2 split IL-4 (“DA”), while the remaining structure constitutes the other part of 2+2 split IL-4 (“BC”) (FIG. 2b). For the split IL-4 PACE approach, the two split IL-4 units were fused with a flexible linker to the C-terminus of traditional PACE molecules. Each molecule (R1, R2, P1, P2) carried one “BC” unit and one “DA” unit.

Conditional cytokine activity required inactivation of the “3+1” and “2+2” versions of IL-4 present in the precursor proteins R1 and R2. It has been described before that the mutations E9Q and R88Q reduce IL-4 activity (Wang Y, Shen B J, Sebald W., Proc Natl Acad Sci USA. 1997; 94(5):1657-1662). Hence, the split IL-4 unit fused to polypeptide chain devoid of a Fab fragment (“dummy chain”) carried either a E9Q mutation (precursor protein R1) or a R88Q mutation (precursor protein R2). Upon polypeptide chain exchange between R1 and R2, the inactive product protein P2 carries both inactivated units, while the activated product protein P1 carries both wildtype units of IL-4, forming a functionally active IL-4 molecule (FIGS. 1b).

TABLE 1
Activatable precursor proteins comprising artificially splitted IL-4

| name | SEQ ID NO:/chain 1 | SEQ ID NO:/chain 2 | SEQ ID NO:/chain 3 | SEQ ID NO:/chain 4 |
|---|---|---|---|---|
| <LeY> IL-4 | 1: [LeY-VH_CH1_Dig- | 2: [LeY-VL_Ckappa] | 3: [Dig-VL_CH3-Hole]- | |
| | VH_CH3-Knob] | | [Linker]-[Human interleukin- | |
| | | | 4] | |
| <LeY> IL-4 DABC | 1: [LeY-VH_CH1_Dig- | 2: [LeY-VL_Ckappa] | 4: [Dig-VL_CH3-Hole]- | |
| | VH_CH3-Knob] | | [Linker]-[Interleukin-4 DABC | |
| | | | (circularly permutated)] | |
| <CD38> R1 3 + 1 E9Q | 5: [CD38-VH_CH1_Dig- | 6: [CD38-VL_Ckappa] | 7: [Biotin-VL_CH3-Hole- | |
| | VH_CH3-Knob]-[Linker]- | | E357K]-[Linker]-[Split | |
| | [Split interleukin-4 (BCD)] | | interleukin-4 (A) E9Q] | |
| <CD38> R2 3 + 1 | 8: [CD38-VH_CH1_Dig- | 6: [CD38-VL_Ckappa] | 9: [Biotin-VH_CH3-Knob- | |
| R88Q | VL_CH3-Hole]-[Linker]- | | K370E]-[Linker]-[Split | |
| | [Split interleukin-4 (A)] | | interleukin-4 (BCD) R88Q] | |
| <Her2> R1 3 + 1 E9Q | 10: [Her2-VH_CH1_Dig- | 11: [Her2-VL_Ckappa] | 7: [Biotin-VL_CH3-Hole- | |
| | VH_CH3-Knob]-[Linker]- | | E357K]-[Linker]-[Split | |
| | [Split interleukin-4 (BCD)] | | interleukin-4 (A) E9Q] | |
| <Her2> R2 3 + 1 | 12: [Her2-VH_CH1_Dig- | 11: [Her2-VL_Ckappa] | 9: [Biotin-VH_CH3-Knob- | |
| R88Q | VL_CH3-Hole]-[Linker]- | | K370E]-[Linker]-[Split | |
| | [Split interleukin-4 (A)] | | interleukin-4 (BCD) R88Q] | |
| <CD38> IL-4 | 13: [CD38-VH_CH1_Dig- | 6: [CD38-VL_Ckappa] | 3: [Dig-VL_CH3-Hole]- | |
| | VH_CH3-Knob] | | [Linker]-[Human interleukin- | |
| | | | 4] | |
| <CD38>P1 3 + 1 | 5: [CD38-VH_CH1_Dig- | 6: [CD38-VL_Ckappa] | 8: [CD38-VH_CH1_Dig- | 6: [CD38- |
| | VH_CH3-Knob]-[Linker]- | | VL_CH3-Hole]-[Linker]- | VL_Ckappa] |
| | [Split interleukin-4 (BCD)] | | [Split interleukin-4 (A)] | |
| <CD38> IL-4 DABC | 13: [CD3 8-VH_CH1_Dig- | 6: [CD38-VL_Ckappa] | 4: [Dig-VL_CH3-Hole]- | |
| | VH_CH3-Knob] | | [Linker]-[Interleukin-4 DABC | |
| | | | (circularly permutated)] | |
| <CD38> R1 2 + 2 E9Q | 14: [CD38-VH_CH1_Dig- | 6: [CD38-VL_Ckappa] | 15: [Biotin-VL_CH3-Hole- | |
| | VH_CH3-Knob]-[Linker]- | | E357K]-[Linker]-[Split | |
| | [Split interleukin-4 (BC)] | | interleukin-4 (DA) E9Q] | |
| <CD38> R2 2 + 2 | 16: [CD38-VH_CH1_Dig- | 6: [CD38-VL_Ckappa] | 17: [Biotin-VH_CH3-Knob- | |
| R88Q | VL_CH3-Hole]-[Linker]- | | K370E]-[Linker]-[Split | |
| | [Split interleukin-4 (DA)] | | interleukin-4 (BC) R88Q] | |
| <CD38> P1 2 + 2 | 14: [CD38-VH_CH1_Dig- | 6: [CD38-VL_Ckappa] | 16: [CD38-VH_CH1_Dig- | 6: [CD38- |
| | VH_CH3-Knob]-[Linker]- | | VL_CH3-Hole]-[Linker]- | VL_Ckappa] |
| | [Split interleukin-4 (BC)] | | [Split interleukin-4 (DA)] | |
| <CD38> R1 3 + 1 no | 5: [CD38-VH_CH1_Dig- | 6: [CD38-VL_Ckappa] | 18: [Biotin-VL_CH3-Hole- | |
| mutation | VH_CH3-Knob]-[Linker]- | | E357K]-[Linker]-[Split | |
| | [Split interleukin-4 (BCD)] | | interleukin-4 (A)] | |
| <CD38> R1 3 + 1 E9A | 5: [CD38-VH_CH1_Dig- | 6: [CD38-VL_Ckappa] | 19: [Biotin-VL_CH3-Hole- | |
| | VH_CH3-Knob]-[Linker]- | | E357K]-[Linker]-[Split | |
| | [Split interleukin-4 (BCD)] | | interleukin-4 (A) E9A] | |
| <CD38> R1 3 + 1 I5A | 5: [CD38-VH_CH1_Dig- | 6: [CD38-VL_Ckappa] | 20: [Biotin-VL_CH3-Hole- | |
| E9Q | VH_CH3-Knob]-[Linker]- | | E357K]-[Linker]-[Split | |
| | [Split interleukin-4 (BCD)] | | interleukin-4 (A) I5A E9Q] | |
| <CD38> R1 3 + 1 T6D | 5: [CD38-VH_CH1_Dig- | 6: [CD38-VL_Ckappa] | 21: [Biotin-VL_CH3-Hole- | |
| E9Q | VH_CH3-Knob]-[Linker]- | | E357K]-[Linker]-[Split | |
| | [Split interleukin-4 (BCD)] | | interleukin-4 (A) T6D E9Q] | |
| <CD38> R1 3 + 1 T6D | 5: [CD38-VH_CH1_Dig- | 6: [CD38-VL_Ckappa] | 22: [Biotin-VL_CH3-Hole- | |
| E9A | VH_CH3-Knob]-[Linker]- | | E357K]-[Linker]-[Split | |
| | [Split interleukin-4 (BCD)] | | interleukin-4 (A) T6D E9A] | |
| <CD38> R2 3 + 1 | 8: [CD38-VH_CH1_Dig- | 6: [CD38-VL_Ckappa] | 23: [Biotin-VH_CH3-Knob- | |
| R88A | VL_CH3-Hole]-[Linker]- | | K370E]-[Linker]-[Split | |
| | [Split interleukin-4 (A)] | | interleukin-4 (BCD) R88A] | |
| <CD38> R2 3 + 1 | 8: [CD38-VH_CH1_Dig- | 6: [CD38-VL_Ckappa] | 24: [Biotin-VH_CH3-Knob- | |
| R81E R88Q | VL_CH3-Hole]-[Linker]- | | K370E]-[Linker]-[Split | |
| | [Split interleukin-4 (A)] | | interleukin-4 (BCD) R81E | |
| | | | R88Q] | |
| <CD38> R2 3 + 1 | 8: [CD38-VH_CH1_Dig- | 6: [CD38-VL_Ckappa] | 25: [Biotin-VH_CH3-Knob- | |
| K84E R88Q | VL_CH3-Hole]-[Linker]- | | K370E]-[Linker]-[Split | |
| | [Split interleukin-4 (A)] | | interleukin-4 (BCD) K84E | |
| | | | R88Q] | |
| <CD38> R2 3 + 1 | 8: [CD38-VH_CH1_Dig- | 6: [CD38-VL_Ckappa] | 26: [Biotin-VH_CH3-Knob- | |
| R88Q N89A | VL_CH3-Hole]-[Linker]- | | K370E]-[Linker]-[Split | |
| | [Split interleukin-4 (A)] | | interleukin-4 (BCD) R88Q | |
| | | | N89A] | |
| <CD38> R2 3 + 1 | 8: [CD38-VH_CH1_Dig- | 6: [CD38-VL_Ckappa] | 27: [Biotin-VH_CH3-Knob- | |
| R88Q W91A | VL_CH3-Hole]-[Linker]- | | K370E]-[Linker]-[Split | |
| | [Split interleukin-4 (A)] | | interleukin-4 (BCD) R88Q | |
| | | | W91A] | |
| <CD38> R1 2 + 2 T6D | 14: [CD38-VH_CH1_Dig- | 6: [CD38-VL_Ckappa] | 28: [Biotin-VL_CH3-Hole- | |
| E9A | VH_CH3-Knob]-[Linker]- | | E357K]-[Linker]-[Split | |
| | [Split interleukin-4 (BC)] | | interleukin-4 (DA) T6D E9A] | |
| <CD38> R2 2 + 2 | 16: [CD38-VH_CH1_Dig- | 6: [CD38-VL_Ckappa] | 29: [Biotin-VH_CH3-Knob- | |
| R81E R88Q | VL_CH3-Hole]-[Linker]- | | K370E]-[Linker]-[Split | |
| | [Split interleukin-4 (DA)] | | interleukin-4 (BC) R81E | |
| | | | R88Q] | |
| <Her2> R1 2 + 2 T6D | 30: [Her2-VH_CH1_Dig- | 11: [Her2-VL_Ckappa] | 28: [Biotin-VL_CH3-Hole- | |
| E9A | VH_CH3-Knob]-[Linker]- | | E357K]-[Linker]-[Split | |
| | [Split interleukin-4 (BC)] | | interleukin-4 (DA) T6D E9A] | |
| <Her2> R2 2 + 2 R81E | 31: [Her2-VH_CH1_Dig- | 11: [Her2-VL_Ckappa] | 29: [Biotin-VH_CH3-Knob- | |
| R88Q | VL_CH3-Hole]-[Linker]- | | K370E]-[Linker]-[Split | |
| | [Split interleukin-4 (DA)] | | interleukin-4 (BC) R81E | |
| | | | R88Q] | |
| <Her2> P1 2 + 2 | 30: [Her2-VH_CH1_Dig- | 11: [Her2-VL_Ckappa] | 31: [Her2-VH_CH1_Dig- | 11: [Her2- |
| | VH_CH3-Knob]-[Linker]- | | VL_CH3-Hole]-[Linker]- | VL_Ckappa] |
| | [Split interleukin-4 (BC)] | | [Split interleukin-4 (DA)] | |

### Example 2

### Design of Activatable Precursor Proteins Comprising IL-2v Bound to an Inactivation Moiety

Presursor proteins of the general antibody domain arrangement illustrated in FIG. 1 were prepared. Two precursor proteins (R1 and R2) contain CH3 domains with knob and hole mutations and further destabilizing mutations. The precursor proteins have a half-IgG-like shape, wherein a non-functional pair of VH/VL doamins is arranged at the N-terminal end of the CH3 domains. The precursor proteins further comprise an Fab fragment specifically binding to LeY.

For this experiment, human interleukin-2 engineered to reduce IL-2 receptor alpha binding (IL-2v) was used (Klein C et al., OncoImmunology, 6:3). IL-2v was fused with a flexible linker to the C-terminus of one polypeptide chain of a precursor protein.

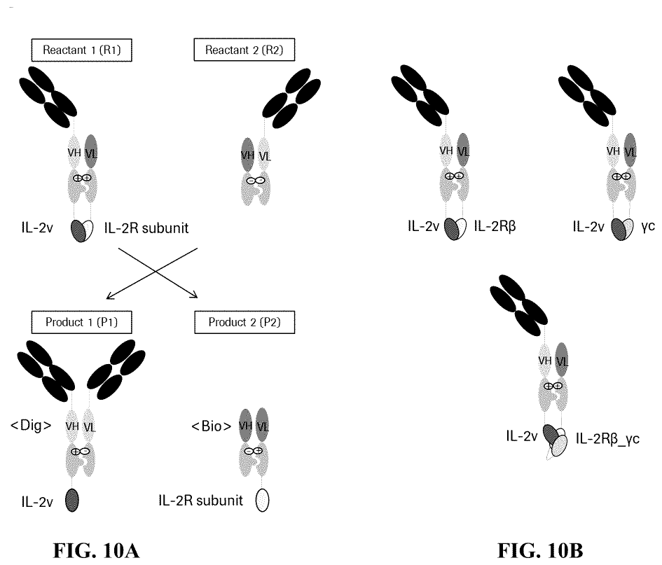

In precursor protein R1, functionally active IL-2v was fused to the C-terminus of one CH3(knob)-polypeptide as indicated in FIG. 10a. The the C-terminus of the CH3(hole)-polypeptide an IL-2 receptor subunit was fused, thereby inactivating the activity of the IL-2v molecule. IL-2v exerts its activity by binding to the cell surface IL-2 receptor, consisting of the two subunits IL-2Rβ and common gamma chain (γc). Hence, inactivation of IL-2v was attempted by fusing the extracellular domain of IL-2Rβ or the extracellular domain of γc or a fusion of IL-2Rβ and γc to the CH3(hole) chain (FIG. 10b). The other precursor protein R2 did not carry any cytokine domains. Consequently, upon polypeptide chain exchange in one of the product proteins, i.e. the activated protein (FIG. 10a), only functionally active IL-2v is comprised. The other, inactive, product polypeptide only comprises the IL-2 receptor subunit.

TABLE 2
Amino acid sequences of activatable precursor proteins comprising IL-2v bound to an inactivation moiety

| name | SEQ ID NO:/chain 1 | SEQ ID NO:/chain 2 | SEQ ID NO:/chain 3 | SEQ ID NO:/chain 4 |
|---|---|---|---|---|
| <LeY> R2 | 32: [LeY-VH_CH1_Dig- | 2: [LeY-VL_Ckappa] | 33: [Biotin-VH_CH3-Knob- | |
| | VL_CH3-Hole] | | K370E] | |
| <LeY> P1 IL-2v | 34: [LeY-VH_CH1_Dig- | 2: [LeY-VL_Ckappa] | 32: [LeY-VH_CH1_Dig- | 2: [LeY-VL_Ckappa] |
| | VH_CH3-Knob]-[Linker]- | | VL_CH3-Hole] | |
| | [Interleukin-2v] | | | |
| <LeY> R1 IL2-Rβ | 34: [LeY-VH_CH1_Dig- | 2: [LeY-VL_Ckappa] | 35: [Biotin-VL_CH3-Hole- | |
| | VH_CH3-Knob]-[Linker]- | | E357K]-[Linker]-[IL-2Rβ | |
| | [Interleukin-2v] | | extracellular domain | |
| | | | (A27-D239)] | |
| <LeY> R1 γc | 34: [LeY-VH_CH1_Dig- | 2: [LeY-VL_Ckappa] | 36: [Biotin-VL_CH3-Hole- | |
| | VH_CH3-Knob]-[Linker]- | | E357K]-[Linker]-[Common | |
| | [Interleukin-2v] | | gamma chain (γc) | |
| | | | extracellular domain | |
| | | | (L23-N254)] | |
| <LeY> R1 IL-2Rβ_γc | 34: [LeY-VH_CH1_Dig- | 2: [LeY-VL_Ckappa] | 37: [Biotin-VL_CH3-Hole- | |
| | VH_CH3-Knob]-[Linker]- | | E357K]-[Linker]-[IL- | |
| | [Interleukin-2v] | | 2Rβ_γc] | |

### Example 3

### Design of Activatable Precursor Proteins Comprising Different Subunits of IL-12

Presursor proteins of the general antibody domain arrangement illustrated in FIG. 1 were prepared. Two precursor proteins (R1 and R2) contain CH3 domains with knob and hole mutations and further destabilizing mutations. The precursor proteins have a half-IgG-like shape, wherein a non-functional pair of VH/VL doamins is arranged at the N-terminal end of the CH3 domains. The precursor proteins further comprise an Fab fragment specifically binding to LeY.

Interleukin-12 (IL-12) shows highest signaling activity as heterodimer of the two disulfide-linked subunits p35 and p40 (Sieburth D et al., Genomics. 1992 September; 14(1):59-62). In order to provide precursor proteins for targeted activation of IL-12, the intermolecular disulfide bond between the two subunits was removed by C73S mutation in p35 and C177S mutation in p40.

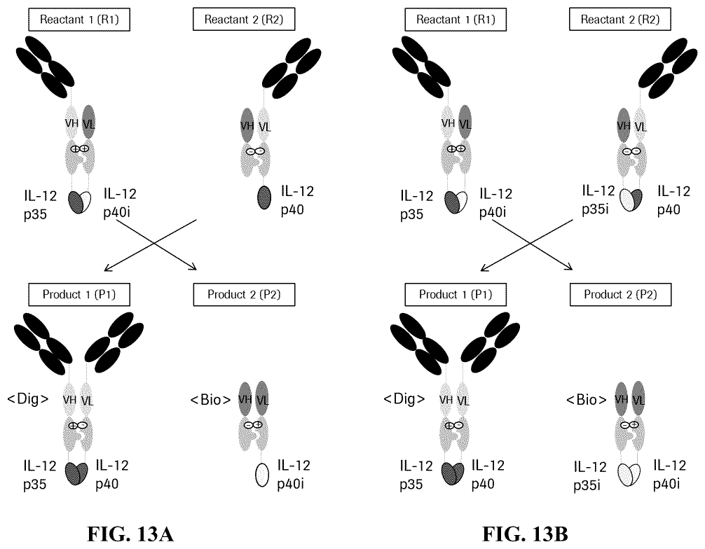

In precursor protein R1, IL-12 subunit p35 was fused to the C-terminus of one CH3(knob)-polypeptide as indicated in FIG. 13a via a flexible linker. The the C-terminus of the CH3(hole)-polypeptide an inactivated variant of IL-12 subunit p40 was fused via a flexible linker, so that overall the IL-12 heterodimer was functionally inactive. In precursor protein R2, a functionally active IL-12 subunit p40 was fused to the C-terminus of the CH3(hole)-polypeptide as indicated in FIG. 13a via a flexible linker.

Upon polypeptide chain exchange between R1 and R2, the activated product protein P1 carries active IL-12 without an inactivation unit (FIG. 13a). The other, inactive, product polypeptide only comprises the inactivated variant of the IL-12 subunit p40.

TABLE 3
Amino acid sequences of activatable precursor proteins comprising different subunits of IL-12

| name | SEQ ID NO:/chain 1 | SEQ ID NO:/chain 2 | SEQ ID NO:/chain 3 | SEQ ID NO:/chain 4 |
|---|---|---|---|---|
| <LeY> P1 IL-12 | 38: [CD38-VH_CH1_Dig- | 2: [LeY-VL_Ckappa] | 39: [LeY-VH_CH1_Dig- | 2: [LeY-VL_Ckappa] |
| | VH_CH3-Knob]-[Linker]-[IL- | | VL_CH3-Hole]-[Linker]-[IL-12 | |
| | 12 p35 (C73S)] | | p40 (C177S)] | |
| <LeY> R1 IL-12 p40i | 40: [LeY-VH_CH1_Dig- | 2: [LeY-VL_Ckappa] | 41: [Biotin-VL_CH3-Hole- | |
| | VH_CH3-Knob]-[Linker]-[IL- | | E357K]-[Linker]-[IL-12 p40i | |
| | 12 p35 (C73S)] | | (C177S)] | |

### Example 4

### Expression and Purification of Precursor Proteins

Expression of the precursor proteins described in Examples 1 to 3 was done by co-transfection of plasmids encoding light chain, heavy chain (with knob or hole-mutations) and matching “dummy” heavy chain (i.e. the heavy-chain-like polypeptide of a precursor protein that was devoid of an antigen binding fragment; hole or knob) into mammalian cells (e.g. HEK293) via state of the art technologies.

In more detail, for example, for the production of the precursor proteins by transient transfection (e.g. in HEK293 cells) expression plasmids based either on a cDNA organization with or without a CMV-Intron A promoter or on a genomic organization with a CMV promoter were applied.

Beside the antibody expression cassettes, the plasmids contained:
an origin of replication, which allows replication of this plasmid in E. coli, and
a β-lactamase gene, which confers ampicillin resistance in E. coli.

The transcription unit of each antibody gene was composed of the following elements:
the immediate early enhancer and promoter from the human cytomegalovirus,
followed by the Intron A sequence in the case of the cDNA organization,
a 5-untranslated region of a human antibody gene,
an immunoglobulin heavy chain signal sequence,
the antibody chain with linker and cytokine or split cytokine sequence either as cDNA or in genomic organization (the immunoglobulin exon-intron organization is maintained), and
a 3-non-translated region with a polyadenylation signal sequence, and

The fusion genes comprising the heavy and light chains were generated by PCR and/or gene synthesis and assembled by known recombinant methods and techniques by connection of the according nucleic acid segments e.g. using unique restriction sites in the respective plasmids. The subcloned nucleic acid sequences were verified by DNA sequencing. For transient transfections larger quantities of the plasmids were prepared by plasmid preparation from transformed E. coli cultures (HiSpeed Plasmid Maxi Kit, Qiagen).

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

The precursor protein were generated by transient transfection with the respective plasmid using the HEK293-F system (Invitrogen) according to the manufacturers instruction. Briefly, HEK293-F cells (Invitrogen) growing in suspension either in a shake flask or in a stirred fermenter in serum-free FreeStyle™ 293 expression medium (Invitrogen) were transfected with the respective expression plasmid and 293fectin™, fectin (Invitrogen) or PEIpro (Polyplus). For 2 L shake flask (Corning) HEK293-F cells were seeded at a density of 1*10⁶ cells/mL in 600 mL and incubated at 120 rpm, 8% CO₂. The day after the cells were transfected at a cell density of approx. 1.5*10⁶ cells/mL with ca. 42 mL mix of A) 20 mL Opti-MEM (Invitrogen) with 300 μg total plasmid DNA (0.5 μg/mL) and B) 20 ml Opti-MEM+1.2 mL 293 fectin or fectin (2 μL/mL) or 750 μl PEIpro (1.25 μL/mL). According to the glucose consumption glucose solution was added during the course of the fermentation. Correctly assembled precursor proteins were secreted into culture supernatants like standard IgGs. The supernatant containing the precursor proteins was harvested after 5-10 days and precursor proteins were either directly purified from the supernatant or the supernatant was frozen at −20° C. and stored.

Because the used precursor proteins contain a kappa light chain they were purified by applying standard kappa light chain affinity chromatography. The precursor proteins were purified from cell culture supernatants by affinity chromatography using KappaSelect (GE Healthcare, Sweden) and Superdex 200 size exclusion (GE Healthcare, Sweden) chromatography or ion exchange chromatography.

Briefly, sterile filtered cell culture supernatants were captured on a KappaSelect resin equilibrated with PBS buffer (10 mM Na₂HPO₄, 1 mM KH₂PO₄, 137 mM NaCl and 2.7 mM KCl, pH 7.4), washed with equilibration buffer and eluted with 50 mM sodium citrate, 150 mM NaCl at pH 3.0. The eluted precursor protein fractions were pooled and neutralized with 2M Tris, pH 9.0. The precursor protein pools were further purified by size exclusion chromatography or ion exchange chromatography. For size exclusion chromatography a Superdex™ 200 pg HiLoad™ 16/600 (GE Healthcare, Sweden) column equilibrated with 20 mM histidine, 140 mM NaCl, pH 6.0. For ion exchange chromatography, the protein sample obtained from KappaSelect purification was diluted 1:10 in 20 mM histidine, pH 6.0 and loaded on a HiTrap™ SP HP ion exchange (GE Healthcare, Sweden) column equilibrated with buffer A (20 mM histidine, pH 6.0). A gradient of 0-100% buffer B (20 mM histidine, 1 M NaCl, pH 6.0) was applied to elute different protein species. The fractions containing the precursor proteins were pooled, concentrated to the required concentration using Vivaspin ultrafiltration devices (Sartorius Stedim Biotech S.A., France) and stored at −80° C.

Purity and integrity were analyzed after purification by SDS-PAGE. Protein solution (13 μl) was mixed with 5 μl 4× NuPAGE LDS sample buffer (Invitrogen) and 2 μl 10× NuPAGE sample reducing agent (Invitrogen) and heated to 95° C. for 5 min. Samples were loaded on a NuPAGE 4-12% Bis-Tris gel (Invitrogen) and run according to the manufacturers instructions using a Novex Mini-Cell (Invitrogen) and NuPAGE MES SDS running buffer (Life Technologies). Gels were stained using InstantBlue™ Coomassie protein stain. Furthermore, integrity and uniformity of proteins was analyzed using analytical size exclusion chromatography.

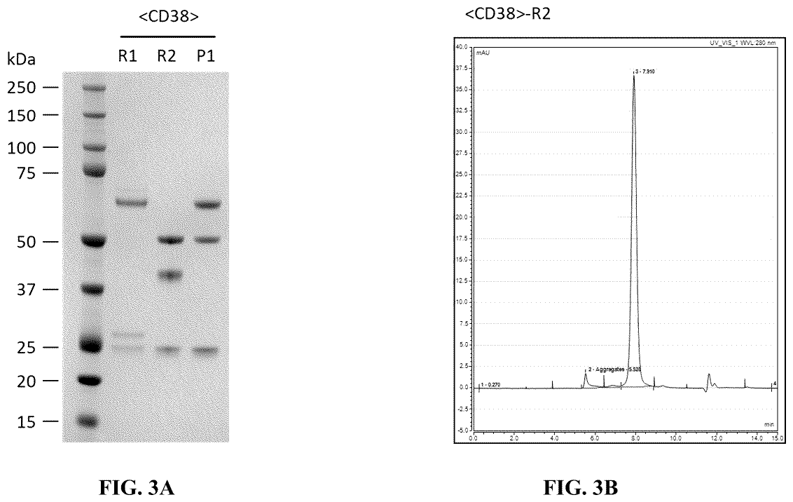

Precursor proteins as described in Example 1 were producible in high purity was as shown in FIGS. 3a and 3b for precursor proteins (R1, R2) and the activated product protein (P1) comprising a Fab fragment specifically binding to CD38. In a similar manner, proteins R1, R2 and P1 comprising a Fab fragment specifically binding to LeY, and proteins R1, R2 and P1 comprising a Fab fragment specifically binding to Her2 were also producible in high purity (data not shown). The proteins were produced successfully, as analyzed by SDS-PAGE and analytical size exclusion chromatography. SDS-PAGE revealed that all expected polypeptide chains were present in the preparations (FIG. 3a); analytical size exclusion confirmed >90% purity of the preparations. For review of methods for assessment of antibody purity, see, e.g., Flatman, S. et al., J. Chrom. B 848 (2007) 79-87. Yields of the preparations were around 0.2 mg/L culture for R1 molecules, 0.5 mg/L culture for R2 molecules and 2 mg/L culture for P1 molecules.

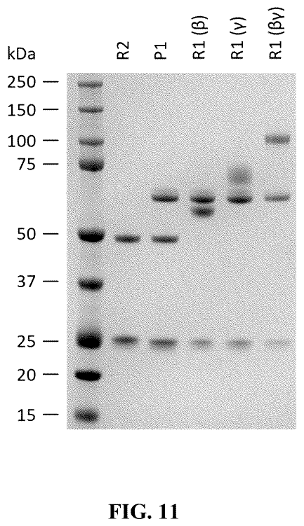

Precursor proteins as described in Example 2 were producible in high purity as shown in FIG. 11 for precursor proteins (R1, R2) and the activated product protein (P1) comprising a Fab fragment specifically binding to LeY. The proteins were produced successfully, as analyzed by SDS-PAGE, revealing that all expected polypeptide chains were present in the preparations (FIG. 11). Yields of the protein preparations were around 10 mg/L culture for R1 molecules, 6 mg/L culture for R2 molecules and 2 mg/L culture for P1 molecules.

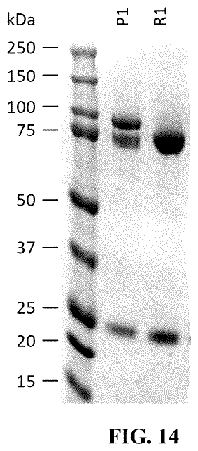

Precursor proteins as described in Example 3 were producible in high purity as shown in FIG. 14 for precursor protein (R1) and the activated product protein (P1) comprising a Fab fragment specifically binding to LeY. The molecules were produced successfully, as analyzed by SDS-PAGE, revealing that all expected polypeptide chains were present in the PI preparation (FIG. 14). For R1, the two different heavy chains had very similar molecular weight, hence not allowing satisfactory differentiation by SDS-PAGE. Instead, mass spectrometry revealed that both heavy chains were present in the R1 preparation. Yields of the preparations were around 5 mg/L culture.

### Example 5

### Cell Surface Targeting of Precursor Proteins Comprising Activatable Splitted IL-4 Generates Activated IL-4

Interleukin-4 activity of precursor proteins of Example 1 was assessed using a TF-1 cell proliferation assay.

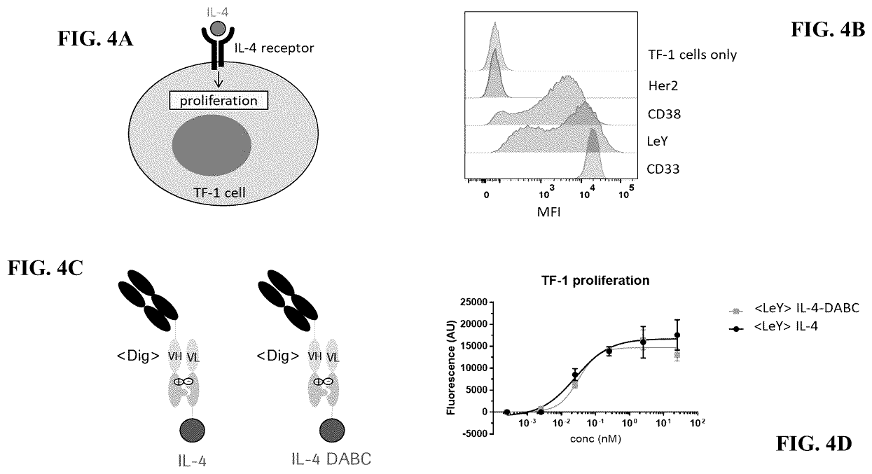

TF-1 cells (originally obtained from Deutsche Sammlung von Mikroorganismen und Zellkulturen GmbH and subsequently adapted to Roche culture conditions) are erythroblast cells that respond to interleukin-4 with increased proliferation (FIG. 4a). The cells were cultured in RPMI-1640 (Gibco, cat. no. A10491-01), 2 ng/ml recombinant human GM-CSF (Abcam), 10% (v/v) fetal bovine serum at 37° C., 5% CO₂. For the assay, TF-1 cells were washed 3× with medium without recombinant human GM-CSF. 20 000 TF-1 cells in medium without recombinant human GM-CSF were added to wells of a 96-well plate. Precursor proteins were added at indicated concentrations, typically ranging from 1 μM to 10 fM. After incubation for 72-96 h at 37° C., 5% CO₂, cell proliferation was assessed using resazurin cell viability assay (Abcam) according to the manufacturers protocol. Briefly, 2× resazurin reagent in pre-warmed PBS was added at 1:1 volume ratio to the cells in the 96-well plate and incubated for up to 4 h at 37° C., 5% CO₂. 100 μl supernatant of the wells was transferred to Corning™ 96-well clear bottom white polystyrene microplates (Thermo Fisher Scientific). Fluorescence was measured at 550 nm excitation and 590 nm emission using an Infinite 200Pro plate reader (Tecan). High fluorescence values correspond to high proliferation. For analysis, fluorescence values of control samples (TF-1 cells without added protein) were set to 0, and the remaining samples adjusted accordingly. Data were analyzed using Prism7 software (GraphPad).

To compare the activity of IL-4 and circularly permutated IL-4 DABC, TF-1 proliferation assay was performed with antibodies fused to either full length IL-4 or full length IL-4 DABC (FIG. 4c).

The activity of both constructs was similar, indicating that circular permutation of IL-4 does not compromise signaling activity (FIG. 4d).

To demonstrate that polypeptide chain exchange between the precursor proeins is enhanced by cell surface targeting, IL4 activity of individual prescursor proteins, precursor protein combinations and activated product proteins was assessed using TF-1 activity assay.

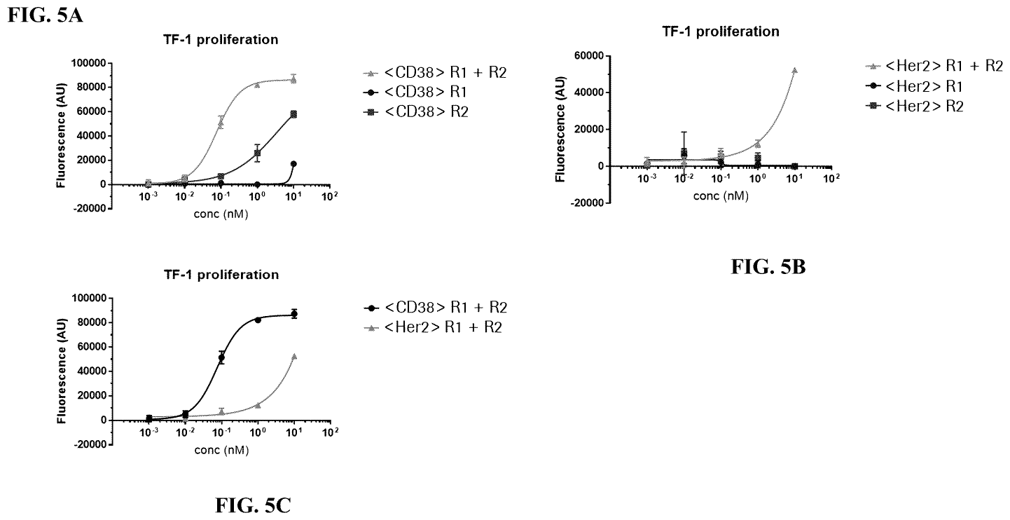

In this assay, activation occurs in cis, i.e. polypeptide chain exchange and IL-4 receptor activation occurs on TF-1 cell surface. The experiments to assess polypeptide chain exchange were carried out in a “targeted setting” using precursor polypeptides each comprising a Fab fragment specifically binding to CD38 that is expressed on TF-1 cells; and as a comparison in a “non-targeted setting” using precursor polypeptides each comprising a Fab fragment specifically binding to Her2 that is not expressed on TF-1 cells (FIG. 4b). Polypeptide chain exchange occurred in both settings, as indicated by the increase in TF-1 proliferation when both split precursor polypeptides “R1+R2” were present as opposed to either R1 or R2 alone (FIG. 5a, 5b). This also shows that one-sided inactivation of split IL-4 by E9Q (R1) and R88Q (R2) was successful.

In direct comparison it is evident that the polypeptide chain exchange in the targeted setting was more efficient than in the non-targeted setting (FIG. 5c). Targeted polypeptide chain exchange occurred already at around 0.1 nM (FIG. 5a), while non-targeted polypeptide chain exchange occurred only at around 10 nM (FIG. 5b). This 100-fold difference between non-targeted and targeted activity indicates that effective generation of functional IL4 from split IL-4 is dependent on effective cell surface targeting. The observed effect of targeting on productive polypeptide chain exchange reflects targeting-induced accumulation of precursor polypeptides, reaching on-cell concentrations that enable effective polypeptide chain exchange. Because polypeptide chain exchange occurs in a concentration and chain accessibility-dependent manner, composition, arrangement and density of target antigens on cell surfaces need to be considered as factors that affects efficacy of polypeptide chain exchange.

### Splitted IL-4 3+1 and 2+2 Comprising Precursor Polypeptides:

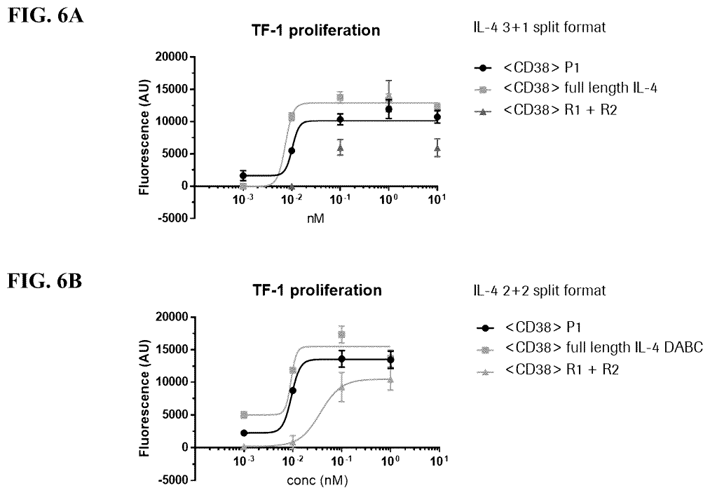

A comparative protein carrying full length IL-4 and the 3+1 split IL-4 activated product protein P1 showed similar activity, indicating that reconstituted 3+1 split IL-4 has similar effector potency as the full length cytokine (FIG. 6a). CD38-targeted polypeptide chain exchange of R1 and R2 reached similar maximal activity as P1 or the comparative protein comprising full length IL-4, indicating that 3+1 split IL-4 can reconstitute full cytokine activity upon polypeptide chain exchange (FIG. 6a).

A comparative protein carrying full length IL-4 DABC or the 2+2 split IL-4 activated product protein P1 showed similar activity, indicating that reconstituted 2+2 split IL-4 has similar effector potency as the full length circularly permutated cytokine (FIG. 6b). CD38-targeted polypeptide chain exchange of R1 and R2 reached similar maximal activity as P1 or full length IL-4, indicating that 2+2 split IL-4 can reconstitute full cytokine activity upon polypeptide chain exchange (FIG. 6b).

### Alternative Version of Splitted IL-4:

To further decrease reactant activity, mutations E9A, 15A E9Q, T6D E9Q and T6D E9A were introduced in the inactivated IL-4 portion of R1; and mutations R88A, R81E R88Q, K84E R88Q, R88Q N89A and R88Q W91A were introduced in the inactivated IL-4 portion of R2.

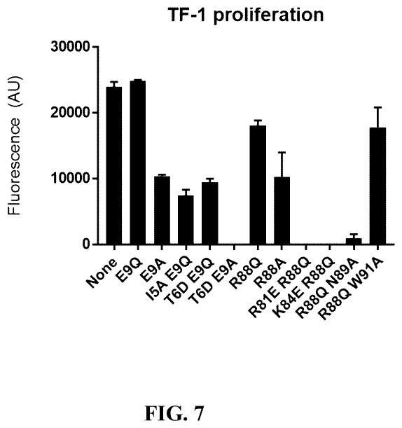

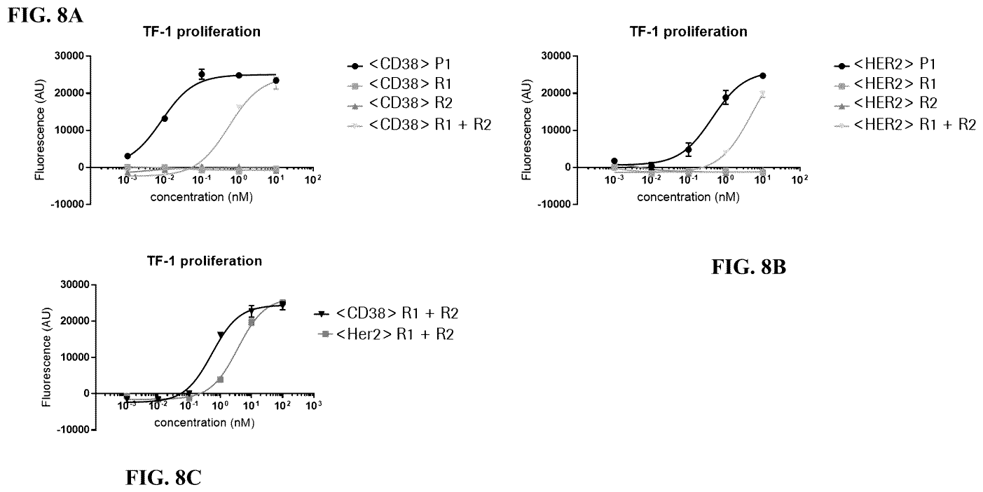

While mutations E9A, 15A E9Q and T6D E9Q that were introduced in the inactivated IL-4 portion of R1 showed reduced residual activity, mutation T6D E9A showed no detectable activity when introduced in the inactivated IL-4 portion of RI (FIG. 7). In the inactived IL-4 portion present in R2, the mutated variants comprising R88A and R88Q N89A substitutions showed reduced residual activity, while mutations R81E R88Q, and K84E R88Q showed no detectable activity (FIG. 7).

### Example 6

### Surface Plasmon Resonance of Precursor Proteins Comprising Activatable Splitted IL-4 Generates Activated IL-4

Signaling activity of interleukin-4 requires binding to the interleukin-4 receptor. Kinetic properties of the interaction between interleukin-4 receptor alpha and proteins R1, R2, P1 and a combination of R1 and R2 containing full length IL-4 (P1) or splitted portions of interleukin-4 was assessed using surface plasmon resonance (SPR).

An anti-histidine antibody (GE Healthcare 28-9980-56) was immobilized in high density (>10.000 RU) on a CM5 sensor. A 5 nM solution of recombinant human IL-4R His-tag protein (Abcam, ab167726) was captured on a CM5 sensor chip for 45 s (capture level ˜55 RU). The interaction with the tested proteins was analyzed by single cycle kinetic at 7.4 nM to 600 nM using 120 s association time and 900 s dissociation time at a flow rate of 50 μl/min. All Biacore T200 experiments were carried out in HBS-P+ (GE Healthcare, Br-1008-27) pH 7.4 running buffer at 25° C. Kinetic properties were determined using T200 evaluation software and the 1:1 Langmuir binding model.

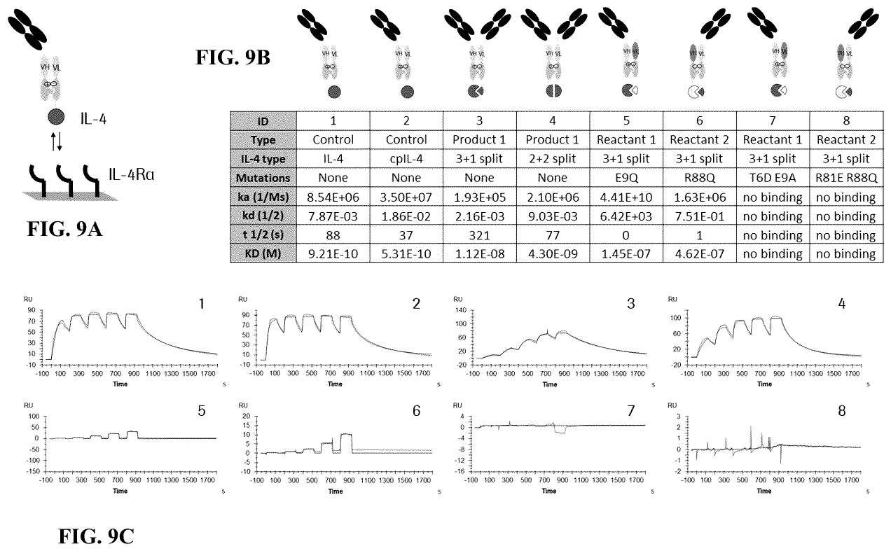

The 3+1 and 2+2 activated product proteins retained binding capabilities to IL-4Rα, although with lower affinity compared to the full length IL-4 control molecules. 3+1 split IL-4 PACE reactant molecules carrying E9Q or R88Q showed residual binding to IL-4Rα, while no binding was detectable for T6D E9A and R81E R88Q variants (FIG. 9b, c). These results are in line with the TF-1 proliferation assay (FIG. 7).

### Example 7

### Cell Surface Targeting of Activatable Precursor Proteins Comprising IL-2v Bound to an Inactivation Moiety

Interleukin-2v activity of precursor proteins of Example 2 was assessed using a CTLL-2 cell proliferation assay.

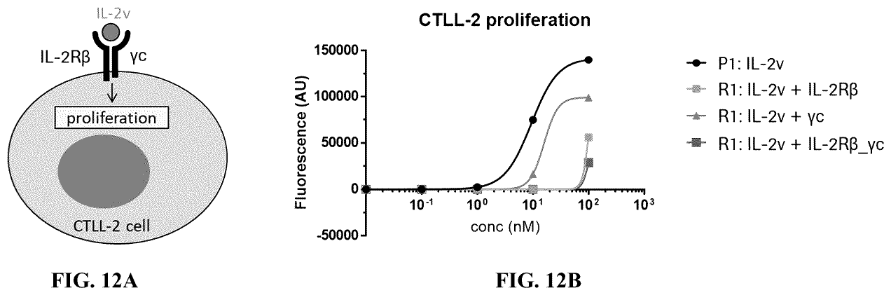

CTLL-2 cells (originally obtained from American Type Culture Collection and subsequently adapted to Roche culture conditions) are murine cytotoxic T lymphocyte cells that respond to interleukin-2v with increased proliferation (FIG. 12a). The cells were cultured in RPMI-1640 (Gibco, cat. no. A10491-01), 10% (v/v) fetal bovine serum, 10% (v/v) T-cell culture supplement with Con A (Becton Dickinson) at 37° C., 5% CO₂. For the assay, CTLL-2 cells were washed 3× with medium without T-cell culture supplement. 30 000 CTLL-2 cells in medium without T-cell culture supplement were added to wells of a U-bottom 96-well plate. Proteins of interest were added at desired concentrations, typically ranging from 1 μM to 10 fM. After incubation for 48-72 h at 37° C., 5% CO₂, cell proliferation was assessed using resazurin cell viability assay (Abcam) according to the manufacturers protocol. Briefly, 2× resazurin reagent in pre-warmed PBS was added at 1:1 volume ratio to the cells in the 96-well plate and incubated for up to 4 h at 37° C., 5% CO₂. 100 μl supernatant of the wells was transferred to Corning™ 96-well clear bottom white polystyrene microplates (Thermo Fisher Scientific). Fluorescence was measured at 550 nm excitation and 590 nm emission using an Infinite 200Pro plate reader (Tecan). High fluorescence values correspond to high proliferation. For analysis, fluorescence values of control samples (CTLL-2 cells without added protein) were set to 0, and the remaining samples adjusted accordingly. Data were analyzed using Prism7 software (GraphPad).

All three different precursor proteins R1, comprising IL-2v in combination with the three different inactivation domains, had reduced IL-2v activities compared to the activated product protein P1 (FIG. 12b).

### Example 8

### Cell Surface Targeting of Activatable Precursor Proteins Comprising Different Subunits of IL-12

Interleukin-12 activity of precursor proteins of Example 3 was assessed using a HEK-Blue™ reporter cell assay according to the manufacturers instructions (Invivogen). HEK-Blue™ IL-12 reporter cells (Invivogen) were cultured in RPMI-1640 (Gibco, cat. no. A10491-01), 10% (v/v) fetal bovine serum, 30 μg/ml blasticidin, 100 μg/ml zeocin at 37° C., 5% CO₂. For the assay, HEK-Blue™ IL-12 reporter cells were washed 3× with medium. 50 000 HEK-Blue™ IL-12 reporter cells in medium were added to wells of a 96-well plate. Proteins of interest were added at desired concentrations, typically ranging from 1 μM to 10 fM. After incubation for 20-24 h at 37° C., 5% CO₂, IL-12 signaling activity was assessed using Quanti-Blue™ (Invivogen) according to the manufacturers protocol. Briefly, the detection reagent was dissolved in pre-warmed endotoxin-free water was instructed and incubated at 37° C. for 30 min. 200 μl detection solution was added to the wells of a Corning™ 96-well clear bottom white polystyrene microplate (Thermo Fisher Scientific) and 20 μl cell supernatant was added. After incubation for 1-5 h at 37° C. absorbance was measured at 640 nm using an Infinite 200Pro plate reader (Tecan). High absorbance values correspond to high IL-12 signaling activity. For analysis, fluorescence values of control samples (HEK-Blue™ IL-12 reporter cells without added protein) were set to 0, and the remaining samples adjusted accordingly. Data were analyzed using Prism7 software (GraphPad).

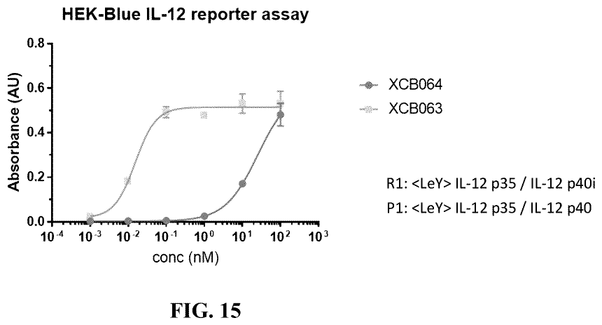

Precursor protein R1 had approximately 1000-fold reduced IL-12 activity compared to the corresponding activated product protein PI (FIG. 15).

### Example 9

### Design of Activatable Precursor Proteins Comprising Artificially Splitted Luciferase

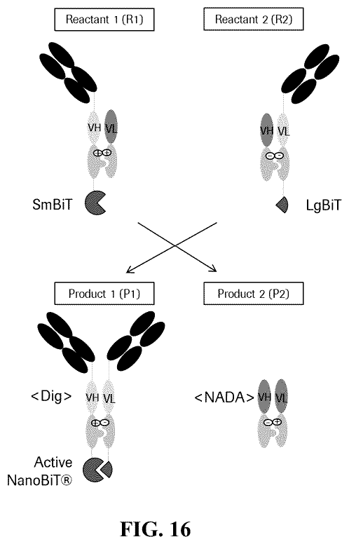

NanoBiT® is a split luciferase enzyme consisting of two subunits, LgBiT and SmBIT (Dixon A S et al., ACS Chem Biol. 2016; 11(2):400-408). When the two subunits come into close proximity, they form a functional enzyme that is able to convert Nano-Glo® Live Cell Substrate, generating a luminescent signal. In order to generate precursor polypeptides, LgBiT and SmBIT were fused with a flexible linker to the C-terminus of the CH3(knob) polypeptide of precursor protein R1 and the CH3(hole) polypeptide of precursor protein R2 (FIG. 16), respectively, following the general structure as indicated for the precursor proteins shown in Example 1. Upon recombination of R1 and R2, the product molecule P1 carries both LgBiT and SmBIT, reconstituting NanoBiT® luciferase activity (FIG. 16).

### Example 10

### Cell Surface Targeting of Split Luciferase PACE Precursors

Activity of proteins containing parts of NanoBiT® (Promega) luciferase as described in Example 9 was assessed using the Nano-Glo® Live Cell Assay System (Promega).

TABLE 4
Amino acid sequences of activatable precursor
proteins comprising split luciferase

| | SEQ ID NO:/ | SEQ ID NO:/ | SEQ ID NO:/ |
|---|---|---|---|
| name | chain 1 | chain 2 | chain 3 |
| <CD38> | 42: [CD38- | 6: [CD38- | 43: [NADA- |
| R1 SmBiT | VH_CH1_Dig- | VL_Ckappa] | VL_CH3-Hole- |
| | VH_CH3-Knob]- | | E357K] |
| | [Linker]-[SmBiT] | | |
| <CD38> | 44: [CD38- | 6: [CD38- | 45: [NADA- |
| R2 LgBiT | VH_CH1_Dig- | VL_Ckappa] | VH_CH3-Knob- |
| | VL_CH3-Hole]- | | K370E] |
| | [Linker]-[LgBiT] | | |

TF-1 cells were cultured in RPMI-1640 (Gibco, cat. no. A10491-01), 2 ng/ml recombinant human GM-CSF (Abcam), 10% (v/v) fetal bovine serum at 37° C., 5% CO₂. For the assay, TF-1 cells were washed 2× with PBS. 100 000 TF-1 cells in Opti-MEM™ I Reduced Serum (Thermo Fisher Scientific) were added to wells of a Corning™ 96-well clear bottom white polystyrene microplate (Thermo Fisher Scientific). A cell-free plate containing only Opti-MEM® I Reduced Serum was prepared to compare on-cell to in-solution PACE. Proteins of interest were added at 50 nM. Luciferase activity was assessed using the Nano-Glo® Live Cell Assay System (Promega) according to the manufacturers protocol. Briefly, Nano-Glo® Live Cell Reagent was added at 1:5 volume ratio and incubated for 1 h at 37° C. Luminescence was measured using an Infinite 200Pro plate reader (Tecan). High luminescence values correspond to high substrate conversion.

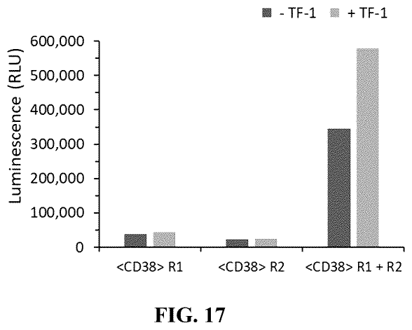

Precursor proteins showed low luminescence, while combinations of the precursor proteins showed high luminescence, indicating successful formation of product molecule P1 (FIG. 17). Targeted polypeptide chain exchange on TF-1 cells showed higher luminescence than non targeted polypeptide chain exchange (without TF-1 cells) (FIG. 17). This difference between non-targeted and targeted activity indicates that effective generation of functional NanoBiT® luciferase from split luciferase PACE molecules is dependent on effective cell surface targeting.

1. A set of a first and a second precursor protein,
wherein each precursor protein comprises two polypeptides that are associated with each other via dimerization domains,
wherein at least one of the precursor proteins comprises a moiety selected from a receptor ligand and an enzyme, wherein said moiety is functionally inactive, wherein said moiety is fused to the dimerization domain,
wherein upon polypeptide chain exchange between the first and the second precursor protein an activated protein is formed,
wherein the activated protein comprises one polypeptide from the first precursor protein and one polypeptides from the second precursor protein, wherein both polypeptides are associated with each other via their dimerization domains, and wherein the activated protein comprises said moiety,
characterized in that the activated protein comprises said moiety in functionally active form.

2. The set of a first and a second precursor protein according to claim 1, wherein either the first precursor protein or the second precursor protein comprise the moiety selected from a receptor ligand and an enzyme, wherein the moiety is bound to an inactivation moiety.

3. The set of a first and a second precursor protein according to claim 2, wherein the moiety is a receptor ligand and the inactivation moiety is the corresponding receptor or a ligand-binding subunit thereof.

4. The set of a first and a second precursor protein according to claim 1, wherein the first precursor protein and the second precursor protein comprise complementary subunits of the moiety selected from a receptor ligand and an enzyme.

5. The set of a first and a second precursor protein according to claim 4, wherein the first precursor protein comprises a first unmodified subunit of the moiety and a second subunit of the moiety, wherein the second subunit comprises an inactivating mutation; and wherein the second precursor protein comprises the second unmodified subunit of the moiety.

6. The set of a first and a second precursor protein according to claim 5, wherein the moiety is a receptor ligand.

7. The set of a first and a second precursor protein according to claim 6, wherein the receptor ligand is a cytokine, wherein the first precursor protein comprises a first subunit of the cytokine and a second subunit of the cytokine, wherein the second subunit comprises an inactivating mutation; and wherein the second precursor protein comprises the second unmodified subunit of the cytokine.

8. The set of a first and a second precursor protein according to claim 1, wherein the first precursor protein and the second precursor protein comprise complementary parts of an artificially splitted moiety selected from a receptor ligand and an enzyme, wherein one of the complementary parts is inactivated.

9. The set of a first and a second precursor protein according to claim 8, wherein the moiety is a receptor ligand.

10. The set of a first and a second precursor protein according to claim 9, wherein the receptor ligand is a cytokine or an enzyme.

11. The set of a first and a second precursor protein according to claim 1, wherein the dimerization domains are CH3 domains.

12. The set of a first and a second precursor protein according to claim 11, wherein the CH3 domains have a modified interface to support polypeptide chain exchange between the first and the second precursor protein.

13. The set of a first and a second precursor protein according to claim 1, wherein the precursor proteins are capable of undergoing polypeptide chain exchange when bound to the surface of a cell.

14. A therapeutic kit comprising a first and a second precursor protein as defined in claim 1.


SEQUENCE LISTING (45 sequences)

SEQ ID NO: 1 - protein, 474 aa, synthetic construct
  REGION 1..474: note = [LeY-VH_CH1_Dig-VH_CH3-Knob]
DVKLVESGGGLVQPGGSLKLSCATSGFTFSDYYMYWVRQTPEKRLEWVAYISNDDSSAAYSDTVKGRFTISRDNARNTLYLQMSRLKSEDTAIYYCARGLAWGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 219 aa, synthetic construct
  REGION 1..219: note = [LeY-VL_Ckappa]
DVLMTQSPLSLPVSLGDQASISCRSSQIIVHSNGNTYLEWYLQKPGQSPKLLIYKVSNRFSGVPDRFSGSGSGTDFTLKISRVEAEDLGVYYCFQGSHVPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 359 aa, synthetic construct
  REGION 1..359: note = [Dig-VL_CH3-Hole]-[Linker]-[Human interleukin-4]
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSHKCDITLQEIIKTLNSLTEQKSTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 4 - protein, 366 aa, synthetic construct
  REGION 1..366: note = [Dig-VL_CH3-Hole]-[Linker]-[Interleukin-4 DABC (circularly permutated)]
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSVKEANQSTLENFLERLKTIMREKYSKCSSGGGSGGSHKCDITLQEIIKTLNSLTEQKSTLCTELTVTDIFAASKNTTEKETFCRAATVLRQFYSHHEKDTRCLGATAQQFHRHKQLIRFLKRLDRNLWGLAGLNSCP

SEQ ID NO: 5 - protein, 600 aa, synthetic construct
  REGION 1..600: note = [CD38-VH_CH1_Dig-VH_CH3-Knob]-[Linker]-[Split interleukin-4 (BCD)]
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKSSS

SEQ ID NO: 6 - protein, 214 aa, synthetic construct
  REGION 1..214: note = [CD38-VL_Ckappa]
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKASTKGPSVFPLAPSSKSTSGGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 250 aa, synthetic construct
  REGION 1..250: note = [Biotin-VL_CH3-Hole-E357K]-[Linker]-[Split interleukin-4 (A) E9Q]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSHKSDITLQQIIKTLNSLTEQK

SEQ ID NO: 8 - protein, 495 aa, synthetic construct
  REGION 1..495: note = [CD38-VH_CH1_Dig-VL_CH3-Hole]-[Linker]-[Split interleukin-4 (A)]
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSDIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSHKSDITLQEIIKTLNSLTEQK

SEQ ID NO: 9 - protein, 350 aa, synthetic construct
  REGION 1..350: note = [Biotin-VH_CH3-Knob-K370E]-[Linker]-[Split interleukin-4 (BCD) R88Q]
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIRFLKRLDQNLWGLAGLNSCPVKEANQSTLENFLERLKTIMREKYSKSSS

SEQ ID NO: 10 - protein, 598 aa, synthetic construct
  REGION 1..598: note = [Her2-VH_CH1_Dig-VH_CH3-Knob]-[Linker]-[Split interleukin-4 (BCD)]
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKSSS

SEQ ID NO: 11 - protein, 214 aa, synthetic construct
  REGION 1..214: note = [Her2-VL_Ckappa]
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 493 aa, synthetic construct
  REGION 1..493: note = [Her2-VH_CH1_Dig-VL_CH3-Hole]-[Linker]-[Split interleukin-4 (A)]
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSDIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSHKSDITLQEIIKTLNSLTEQK

SEQ ID NO: 13 - protein, 477 aa, synthetic construct
  REGION 1..477: note = [CD38-VH_CH1_Dig-VH_CH3-Knob]
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 571 aa, synthetic construct
  REGION 1..571: note = [CD38-VH_CH1_Dig-VH_CH3-Knob]-[Linker]-[Split interleukin-4 (BC)]
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIRFLKRLDRNLWGLAGLNSCP

SEQ ID NO: 15 - protein, 286 aa, synthetic construct
  REGION 1..286: note = [Biotin-VL_CH3-Hole-E357K]-[Linker]-[Split interleukin-4 (DA) E9Q]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSVKEANQSTLENFLERLKTIMREKYSKCSSGGGSGGSHKCDITLQQIIKTLNSLTEQK

SEQ ID NO: 16 - protein, 531 aa, synthetic construct
  REGION 1..531: note = [CD38-VH_CH1_Dig-VL_CH3-Hole]-[Linker]-[Split interleukin-4 (DA)]
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSDIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSVKEANQSTLENFLERLKTIMREKYSKCSSGGGSGGSHKCDITLQEIIKTLNSLTEQK

SEQ ID NO: 17 - protein, 321 aa, synthetic construct
  REGION 1..321: note = [Biotin-VH_CH3-Knob-K370E]-[Linker]-[Split interleukin-4 (BC) R88Q]
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIRFLKRLDQNLWGLAGLNSCP

SEQ ID NO: 18 - protein, 250 aa, synthetic construct
  REGION 1..250: note = [Biotin-VL_CH3-Hole-E357K]-[Linker]-[Split interleukin-4 (A)]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSHKSDITLQEIIKTLNSLTEQK

SEQ ID NO: 19 - protein, 250 aa, synthetic construct
  REGION 1..250: note = [Biotin-VL_CH3-Hole-E357K]-[Linker]-[Split interleukin-4 (A) E9A]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSHKSDITLQAIIKTLNSLTEQK

SEQ ID NO: 20 - protein, 250 aa, synthetic construct
  REGION 1..250: note = [Biotin-VL_CH3-Hole-E357K]-[Linker]-[Split interleukin-4 (A) I5A E9Q]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSHKSDATLQQIIKTLNSLTEQK

SEQ ID NO: 21 - protein, 250 aa, synthetic construct
  REGION 1..250: note = [Biotin-VL_CH3-Hole-E357K]-[Linker]-[Split interleukin-4 (A) T6D E9Q]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSHKSDIDLQQIIKTLNSLTEQK

SEQ ID NO: 22 - protein, 250 aa, synthetic construct
  REGION 1..250: note = [Biotin-VL_CH3-Hole-E357K]-[Linker]-[Split interleukin-4 (A) T6D E9A]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSHKSDIDLQAIIKTLNSLTEQK

SEQ ID NO: 23 - protein, 350 aa, synthetic construct
  REGION 1..350: note = [Biotin-VH_CH3-Knob-K370E]-[Linker]-[Split interleukin-4 (BCD) R88A]
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIRFLKRLDANLWGLAGLNSCPVKEANQSTLENFLERLKTIMREKYSKSSS

SEQ ID NO: 24 - protein, 350 aa, synthetic construct
  REGION 1..350: note = [Biotin-VH_CH3-Knob-K370E]-[Linker]-[Split interleukin-4 (BCD) R81E R88Q]
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIEFLKRLDQNLWGLAGLNSCPVKEANQSTLENFLERLKTIMREKYSKSSS

SEQ ID NO: 25 - protein, 350 aa, synthetic construct
  REGION 1..350: note = [Biotin-VH_CH3-Knob-K370E]-[Linker]-[Split interleukin-4 (BCD) K84E R88Q]
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIRFLERLDQNLWGLAGLNSCPVKEANQSTLENFLERLKTIMREKYSKSSS

SEQ ID NO: 26 - protein, 350 aa, synthetic construct
  REGION 1..350: note = [Biotin-VH_CH3-Knob-K370E]-[Linker]-[Split interleukin-4 (BCD) R88Q N89A]
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIRFLKRLDQALWGLAGLNSCPVKEANQSTLENFLERLKTIMREKYSKSSS

SEQ ID NO: 27 - protein, 350 aa, synthetic construct
  REGION 1..350: note = [Biotin-VH_CH3-Knob-K370E]-[Linker]-[Split interleukin-4 (BCD) R88Q W91A]
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIRFLKRLDQNLAGLAGLNSCPVKEANQSTLENFLERLKTIMREKYSKSSS

SEQ ID NO: 28 - protein, 286 aa, synthetic construct
  REGION 1..286: note = [Biotin-VL_CH3-Hole-E357K]-[Linker]-[Split interleukin-4 (DA) T6D E9A]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSVKEANQSTLENFLERLKTIMREKYSKCSSGGGSGGSHKCDIDLQAIIKTLNSLTEQK

SEQ ID NO: 29 - protein, 321 aa, synthetic construct
  REGION 1..321: note = [Biotin-VH_CH3-Knob-K370E]-[Linker]-[Split interleukin-4 (BC) R81E R88Q]
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIEFLKRLDQNLWGLAGLNSCP

SEQ ID NO: 30 - protein, 569 aa, synthetic construct
  REGION 1..569: note = [Her2-VH_CH1_Dig-VH_CH3-Knob]-[Linker]-[Split interleukin-4 (BC)]
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTLCTELTVTDIFAASKNTTEKETFCRAATVLRQFYSHHEKDTRCLGATAQQFHRHKQLIRFLKRLDRNLWGLAGLNSCP

SEQ ID NO: 31 - protein, 529 aa, synthetic construct
  REGION 1..529: note = [Her2-VH_CH1_Dig-VL_CH3-Hole]-[Linker]-[Split interleukin-4 (DA)]
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSDIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSVKEANQSTLENFLERLKTIMREKYSKCSSGGGSGGSHKCDITLQEIIKTLNSLTEQK

SEQ ID NO: 32 - protein, 456 aa, synthetic construct
  REGION 1..456: note = [LeY-VH_CH1_Dig-VL_CH3-Hole]
DVKLVESGGGLVQPGGSLKLSCATSGFTFSDYYMYWVRQTPEKRLEWVAYISNDDSSAAYSDTVKGRFTISRDNARNTLYLQMSRLKSEDTAIYYCARGLAWGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSDIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 33 - protein, 227 aa, synthetic construct
  REGION 1..227: note = [Biotin-VH_CH3-Knob-K370E]
QVQLVQSGAEVKKPGSSVKVSCKSSGFNNKDTFFQWVRQAPGQGLEWMGRIDPANGFTKYAQKFQGRVTITADTSTSTAYMELSSLRSEDTAVYYCARWDTYGAAWFAYWGQGTLVTVSSGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 623 aa, synthetic construct
  REGION 1..623: note = [LeY-VH_CH1_Dig-VH_CH3-Knob]-[Linker]-[Interleukin- 2v]
DVKLVESGGGLVQPGGSLKLSCATSGFTFSDYYMYWVRQTPEKRLEWVAYISNDDSSAAYSDTVKGRFTISRDNARNTLYLQMSRLKSEDTAIYYCARGLAWGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSAPASSSTKKTQLQLEHLLLDLQMILNGINNYSKNPKLTRMLTAKFAMPKKATELKHLQCLEEELKPLEEVLNGAQSKNFHLRPRDLISNINVIVLELKGSETTFMCEYADETATIVEFLNRWITFSQSIISTLT

SEQ ID NO: 35 - protein, 442 aa, synthetic construct
  REGION 1..442: note = [Biotin-VL_CH3-Hole-E357K]-[Linker]-[IL-2R? extracellular domain (A27 - D239)]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 36 - protein, 461 aa, synthetic construct
  REGION 1..461: note = [Biotin-VL_CH3-Hole-E357K]-[Linker]-[Common gamma chain (?c) extracellular domain (L23 - N254)]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKEN

SEQ ID NO: 37 - protein, 689 aa, synthetic construct
  REGION 1..689: note = [Biotin-VL_CH3-Hole-E357K]-[Linker]-[IL-2R?_?c]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDGGGGSGGGGSGGGGSLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKEN

SEQ ID NO: 38 - protein, 689 aa, synthetic construct
  REGION 1..689: note = [CD38-VH_CH1_Dig-VH_CH3-Knob]-[Linker]-[IL-12 p35 (C73S)]
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 39 - protein, 777 aa, synthetic construct
  REGION 1..777: note = [LeY-VH_CH1_Dig-VL_CH3-Hole]-[Linker]-[IL-12 p40 (C177S)]
DVKLVESGGGLVQPGGSLKLSCATSGFTFSDYYMYWVRQTPEKRLEWVAYISNDDSSAAYSDTVKGRFTISRDNARNTLYLQMSRLKSEDTAIYYCARGLAWGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSDIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 40 - protein, 686 aa, synthetic construct
  REGION 1..686: note = [LeY-VH_CH1_Dig-VH_CH3-Knob]-[Linker]-[IL-12 p35 (C73S)]
DVKLVESGGGLVQPGGSLKLSCATSGFTFSDYYMYWVRQTPEKRLEWVAYISNDDSSAAYSDTVKGRFTISRDNARNTLYLQMSRLKSEDTAIYYCARGLAWGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSRNLPVATPDPGMFPCLHHSQNLLRAVSNMLQKARQTLEFYPCTSEEIDHEDITKDKTSTVEACLPLELTKNESSLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 41 - protein, 445 aa, synthetic construct
  REGION 1..445: note = [Biotin-VL_CH3-Hole-E357K]-[Linker]-[IL-12 p40i (C177S)]
DIQMTQSPSSLSASVGDRVTITCRASGNIHNYLSWYQQKPGKVPKLLIYSAKTLADGVPSRFSGSGSGTDFTLTISSLQPEDVATYYCQHFWSSIYTFGQGTKLEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSSTDILKDQKEPKNKTFLRCEAKNYSGRFTCWWLTTISTDLTFSVKSSRGSSDPQGVTCGAATLSAERVRGDNKEYEYSVECQEDSASPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 42 - protein, 503 aa, synthetic construct
  REGION 1..503: note = [CD38-VH_CH1_Dig-VH_CH3-Knob]-[Linker]-[SmBiT]
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSSGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSVTGYRLFEEIL

SEQ ID NO: 43 - protein, 214 aa, synthetic construct
  REGION 1..214: note = [NADA-VL_CH3-Hole-E357K]
DIQMTQSPSSLSASVGDRVTITCRASQDVGSGVAWYQQKPGKAPKLLIGSASGLYSGVPSRFSGSRSGTDFTLTISSLQPEDFATYYCQQSASGGSTFGQGTKVEIKGQPREPQVYTLPPSRDKLTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 633 aa, synthetic construct
  REGION 1..633: note = [CD38-VH_CH1_Dig-VL_CH3-Hole]-[Linker]-[LgBiT]
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSDIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSMVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 45 - protein, 227 aa, synthetic construct
  REGION 1..227: note = [NADA-VH_CH3-Knob-K370E]
EVQLVESGGGLVQPGGSLRLSCAASGFSIAGTAIHWVRQAPGKGLEWVASISPGGGSTAYADSVKGRFTISADTSKNTAYLQMNSLRAEDTAVYYCSRSGGSGASAMDYWGQGTLVTVSSGQPREPQVYTLPPSRDELTKNQVSLWCLVEGFYPSDIAVEWESNGQPENNYKTTPPVLDSDGSFFLYSKLTVDKSRWQQGNVFSCSVMHEALHNHYTQKSLSLSPGK